SEQUENCE LISTING (5 sequences)

SEQ ID NO: 1 - DNA, 20 bp, Artificial Sequence
  Description of Artificial Sequence Synthetic primer
ccctgcggagtgtgagaagt

SEQ ID NO: 2 - DNA, 20 bp, Artificial Sequence
  Description of Artificial Sequence Synthetic primer
tgcttctgcctcagctgcct

SEQ ID NO: 3 - DNA, 20 bp, Artificial Sequence
  Description of Artificial Sequence Synthetic primer
ctcttccagccttccttcct

SEQ ID NO: 4 - DNA, 20 bp, Artificial Sequence
  Description of Artificial Sequence Synthetic primer
agcactgtgttggcgtacag

SEQ ID NO: 5 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

(19) United States
(12) Patent Application Publication
(10) Pub. No.: US 2009/0124631 A1
(43) Pub. Date: May 14, 2009

(54) Combination Therapy

(76) Inventors: Zuomei Li, Kirkland (CA); Koji Murakami, Tokorozawa (JP)

Correspondence Address: KEOWN & ZUCCHERO, LLP, 500 WEST CUMMINGS PARK, SUITE 1200, WOBURN, MA, 01801, US

(21) Appl. No.: 12/209,419

(22) Filed: Sep. 12, 2008

Related U.S. Application Data
(60) Provisional application No. 60/972,353, filed on Sep. 14, 2007.
(60) Provisional application No. 61/043,957, filed on Apr. 10, 2008.

(51) Int. Cl. A61K 31/506; A61P 35/00; A61K 31/4439; A61K 31/337; A61K 31/40; A61K 31/4468; A61K 31/496; A61K 31/427

(52) U.S. Cl. 514/253.01; 514/275; 514/343; 514/449; 514/426; 514/329; 514/365

(57) ABSTRACT

The invention relates to the treatment of mammalian diseases manifested by abnormal cell growth and/or abnormal cell proliferation. More particularly, the invention relates to the use of combination therapies to control abnormal cell growth and/or abnormal cell proliferation. In particular, the invention relates to the use of isotype-selective inhibitors of histone deacetylases 1, 2 and/or 3 (HDACs 1-3), as well as isotype-selective inhibitors of HDAC1 and/or HDAC2, to potentiate therapeutic activity of microtubule-stabilization agents.

### RELATED APPLICATIONS

This application claims the benefit of U.S. Provisional Application Ser. No. 60/972,353, filed Sep. 14, 2007 and U.S. Provisional Application Ser. No. 61/043,957, filed Apr. 10, 2008. The entire teachings of the above-referenced applications are incorporated herein by reference.

### BACKGROUND OF THE INVENTION

1. Field of the Invention

The invention relates to the treatment of mammalian disease manifested by abnormal cell growth and/or abnormal cell proliferation. More particularly, the invention relates to the use of combination therapies to control abnormal cell growth and/or abnormal cell proliferation.

2. Summary of the Related Art

Histone deacetylases play an important role in gene regulation in mammalian cells. Gray and Ekstrom, Expr. Cell. Res. 262: 75-83 (2001); Zhou et al., Proc. Natl. Acad. Sci. USA 98: 10572-10577 (2001); Kao et al. J. Biol. Chem. 277: 187-193 (2002) and Gao et al. J. Biol. Chem. 277: 25748-25755 (2002) teach that there are 11 members of the histone deacetylase (HDAC) family.

The role of HDACs in transcription and its link to disease has recently been explored. Minnucci et al., Proc. Natl. Acad. Sci. USA 94: 11295-11300 (1997); Hassig et al., Chem. Biol. 4: 783-789 (1998); Grignani et al., Nature 391: 815-818 (1998) and Siddique et al., Oncogene 16: 2283-2285 (1998) suggest that inhibitors of HDACs may be useful for transcription therapy in various human diseases. US Patent Application Publication 2006/0058298 discloses various histone deacetylase inhibitors and methods for their use.

Non-selective inhibitors of histone deacetylases, such as SAHA, TSA or NVP-LAQ824, are not only inhibitors of deacetylases of class I (HDAC1, 2, 3, 8), but also inhibitors of class II (such as HDAC6). Inhibition of HDAC6 leads to tubulin acetylation, a process that can change the stability of microtubules. Matsuyama et al., The EMBO Journal 21: 6820-6831 (2002), teaches that HDAC6 plays a key regulatory role in the stability of microtubules.

Taxanes are a commonly used chemotherapeutic. Taxanes interact with polymerized tubulin to cause microtubule stabilization, resulting in cells becoming unable to resolve the mitotic spindle and undergoing mitotic arrest or apoptosis.

### BRIEF SUMMARY OF THE INVENTION

The invention provides a new approach to the therapeutic treatment of disease manifested by abnormal cell growth and/or abnormal cell proliferation. The present inventors have surprisingly discovered that isotype-selective inhibitors of histone deacetylases 1, 2 and/or 3 (HDACs 1-3), as well as isotype-selective inhibitors of HDAC1 and/or HDAC2, significantly potentiates therapeutic activity of microtubule-stabilization agents, such as taxane compounds.

In a first aspect, the invention provides a method for inhibiting abnormal cell growth and/or abnormal cell proliferation in a mammal, the method comprising administering to a mammal in need thereof an effective amount of a selective inhibitor of histone deacetylase (HDAC)1, HDAC2 and/or HDAC3 in combination with an effective amount of a compound that stabilizes microtubules.

In a second aspect, the invention provides a method for inhibiting abnormal cell growth and/or abnormal cell proliferation in a mammal, the method comprising administering to a mammal in need thereof an effective amount of a selective inhibitor of histone deacetylase (HDAC)1 and/or HDAC2 in combination with an effective amount of a compound that stabilizes microtubules.

In a third aspect, the invention provides a method for inhibiting abnormal cell growth and/or abnormal cell proliferation in a mammal, the method comprising up-regulating the expression of metalothionene 3 (MT3) in the cell and/or up-regulating the expression of thrombospondin-1 (TSP1) in the cell, in combination with administering a compound that stabilizes microtubules.

In a fourth aspect, the invention provides a method for inhibiting abnormal cell growth and/or abnormal cell proliferation in a mammal, the method comprising administering to a mammal in need thereof an agonist of TSP1 receptor in combination with a compound that stabilizes microtubules.

In a fifth aspect, the invention provides a method for inhibiting abnormal cell growth and/or abnormal cell proliferation in a mammal, the method comprising up-regulating the expression of thrombospondin-1 (TSP1) in the cell, in combination with administering a compound that stabilizes microtubules.

In a sixth aspect, the invention provides a method for inhibiting abnormal cell growth and/or abnormal cell proliferation in a mammal, the method comprising administering to a mammal in need thereof an agonist of metalothionene 3 (MT3) expression in the cell and/or an agonist of thrombospondin-1 (TSP1) expression in the cell, in combination with administering a compound that stabilizes microtubules.

In a seventh aspect, the invention provides a method for inhibiting angiogenesis, the method comprising administering to a mammal a selective inhibitor of histone deacetylase (HDAC)1, HDAC2 and/or HDAC3.

In an eighth aspect, the invention provides a method for inducing expression of an anti-angiogenesis factor in a cell, the method comprising administering to the cell a selective inhibitor of histone deacetylase (HDAC)1, HDAC2 and/or HDAC3.

In a ninth aspect, the invention provides a method for inhibiting expression of an angiogenesis factor in a cell, the method comprising administering to the cell a selective inhibitor of histone deacetylase (HDAC)1, HDAC2 and/or HDAC3.

In a tenth aspect, the invention provides a method for controlling abnormal cell growth and/or abnormal cell proliferation in a patient comprising administering to the patient an effective amount of a selective inhibitor of histone deacetylase (HDAC)1, HDAC2 and/or HDAC3 in combination with an effective amount of a compound that stabilizes microtubules.

In an eleventh aspect, the invention provides a method for controlling abnormal cell growth and/or abnormal cell proliferation in a patient comprising administering to a patient in need thereof an effective amount of a selective inhibitor of histone deacetylase (HDAC)1 and/or HDAC2 in combination with an effective amount of a compound that stabilizes microtubules.

In a twelfth aspect, the invention provides the use of a selective inhibitor of histone deacetylase (HDAC)1, HDAC2 and/or HDAC3, preferably a selective inhibitor of HDAC1 and/or HDAC2, in combination with a compound that stabilizes microtubules for the manufacture of a medicament to inhibit abnormal cell growth and/or abnormal cell proliferation or to otherwise treat cancer in a patient.

### BRIEF DESCRIPTION OF THE DRAWINGS

FIG. 1 shows dose-dependent induction of histone H3 acetylation (A) but not tubulin acetylation (B) by Compound A in human bladder carcinoma T24 cells in vitro. Nonselective effect of SAHA and NVP-LAQ824 on histone H3 and tubulin acetylation is also shown. Acetylation was determined by using ELISA.

FIG. 2 shows that in human prostate cancer Du145 cells, inhibition of bFGF transcription by Compound A is more dramatic than by SAHA both at 3 μM after 24 hour treatment.

FIG. 3 shows that Compound A inhibits tubule length in co-cultured human endothelial cells in a dose-dependent manner.

FIG. 4 shows induction of TSP-1 transcription in mouse stromal cells in implanted H460 tumors from mice treated with Compound A (100 mg/kg) and Compound B (40 mg/kg) by 5 repeated dose of oral administration. Three tumors from each treatment group were harvested and analyzed by cDNA array and average values are shown.

FIG. 4A shows induction of transcription of anti-angiogenesis genes in colon adenocarcinoma HCT15 cells by Compound A using microarray analysis. The results indicate the fold induction in treated samples compared to non-treated samples (average of three biological replicates ±standard deviation.

FIG. 5 shows a growth response curve of mouse endothelial cells (MS-1) in the presence or absence of recombinant TSP-1 (10 μg/ml) in culture.

FIG. 6 shows induction of TSP-1 (THBS1) transcription in human cancer HCT15 cells in vitro by Compound A and B by microarray analysis

FIG. 7 shows induction of MT3 transcription in human colon cancer HCT15 cells by 1 uM of Compound A, SAHA, Compound B or Compound C. Compound A is much more potent than SAHA to induce MT3 transcription. The ability of Compound A to induce MT3 expression is dependent on HDAC inhibition.

FIG. 8 shows induction of MT3 transcription in human colon cancer HCT15 cells by 1 uM of Compound D by microarray analysis.

FIG. 9 shows that dose-dependent induction of MT3 Transcription by Compound A in human T-cell leukemia Jurkat-T cells, and human myeloma RPMI-8226 cells in vitro using real time RT-PCR. Cells were treated with various doses of Compound A for 24 hours before RNAs were extracted and analyzed.

FIG. 10 shows induction of MT3 transcription in implanted H460 tumors in vivo in mice treated with a single dose of Compound A (100 mg/kg, po). Transcription of MT3 was analyzed by real time RT-PCR.

FIG. 11 (A) shows the relative transcription level of MT3 in human cancer HCT15 cells transfected with an empty vector (control) and in three clones of human cancer HCT15 cells stably transfected with MT3 expression vector (clone #3-1, #4-4, #5-4) or by using real time RT-PCR; (B) shows the growth curve of the three clones of HCT15 cells, as well as the control HCT15 cells; (C) shows the apoptosis of three clones and the control HCT15 cells monitored by ELISA; (D) shows that overexpression of MT3 blocks the anchorage-independent growth of HCT15 colon cancer cell clones which overexpress MT3 in soft agar.

FIG. 12 shows IC50's (μM) of a panel of cytotoxic agents in human colon cancer HCT15 cells stably transfected with either empty vector (HCT15-control) or with MT3 expression vector (clone #5-4). Overexpression of MT3 specifically sensitized HCT15 cancer cells to both taxotere and taxol, but not other agents.

FIG. 13 shows tumor volume (A) and percentage of body weight change (B) of nude mice bearing human non-small lung H460 tumors after treatment by oral administration with Compound A (25 mg/kg) alone, taxol (TXL, 60 mg/kg, i.v.) alone, or the two agents in combination in vivo. Schedule of combination treatment is shown in (C). Compound A was administered 3 times per week (day 1, 3 and 5 within each week), while taxol was administered once per two weeks (day 1 and day 15). Experiment was terminated after 29 days.

FIG. 14 shows tumor volume (A) and percentage of body weight change (B) of nude mice bearing human non-small lung H460 tumors after treatment by oral administration with Compound B (10 mg/kg) alone, taxol (60 mg/kg, i.v.) alone, or the two agents in combination in vivo. Schedule of combination treatment is described in FIG. 13C.

FIG. 15 shows tumor volume of nude mice bearing human prostate Du145 tumors after treatment by oral administration with Compound A at 50 mg/kg (A) or Compound B at 20 mg/kg (B) with taxol (60 mg/kg, i.v.) combination in vivo. Tumor weights of treated mice are shown in (C). The schedule of combination treatment is described in FIG. 13C.

FIG. 16 shows tumor volume (A) and percentage of body weight change (B) of nude mice bearing human AZ521 stomach tumors after treatment by oral administration with Compound A (150 mg/kg) alone, taxol (20 mg/kg, i.v.) alone, or the two agents in combination in vivo. Schedule of combination is shown in (C).

FIG. 17 shows tumor volume (A) and percentage of body weight change (B) of nude mice bearing human TSU-Pr1 prostate tumors after treatment by oral administration with Compound A (25 mg/kg) alone, taxol (60 mg/kg, i.v.) alone, or the two agents in combination in vivo. Schedule of combination is shown in (C), where taxol was dosed on 1^{st} day and Compound A was dosed three times weekly for 2 weeks.

FIG. 18 shows tumor volume (A) and percentage of body weight change (B) of nude mice bearing human non-small cell lung H460 tumors after treatment with Compound A (40 mg/kg, i.v.) alone, taxol (60 mg/kg, i.v.) alone, or the two agents in combination in vivo. Both drugs were used as a single dose on day 1 and experiment was ended on day 15, as shown in (C).

FIG. 19 shows tumor volume (A) and percentage of body weight change (B) of nude mice bearing human non-small cell lung H460 tumors after treatment by oral administration with Compound A (30 mg/kg) alone, taxotere (TXT, i.v., 30 mg/kg) alone, or two agents in combination in vivo. Compound A was administered three times per week for three weeks, while taxotere was administered once at the beginning of the experiment, as shown in (C).

FIG. 20 shows tumor volume (A) and percentage of body weight change (B) of nude mice bearing human non-small cell lung H460 tumors after treatment by oral administration with Compound A (100 mg/kg) alone, taxotere (TXT, i.v., 30 mg/kg) alone, and two agents in combination in vivo. Compound A was administered three times per week for three weeks, while taxotere was administered once on day 8, as shown in (C).

FIG. 21 shows tumor volume (A) of nude mice bearing human AZ521 stomach tumors after treatment by oral administration with Compound D (40 mg/kg) alone, taxol (TXL, 20 mg/kg, i.v.) alone, or the two agents in combination in vivo. Schedule of combination is shown in (B), where Compound D was dosed once daily for 14 days and taxol was dosed as a single administration on day 1.

FIG. 22 shows tumor weight of nude mice bearing human Du145 prostate tumors after treatment by oral administration with Compound D (10 mg/kg, 20 mg/kg or 40 mg/kg) alone, taxol (60 mg/kg, i.v.) alone, or the two agents in combination in vivo. Compound D was dosed once daily for 14 days and taxol was dosed as a single administration on day 1.

FIG. 23 shows tumor volume (A) and percentage of body weight change of nude mice bearing human H460 non-small cell lung tumors after treatment by oral administration with Compound E (40 mg/kg, or 80 mg/kg) alone, taxol (TXL, 60 mg/kg, i.v.) alone, or the two agents in combination in vivo. Compound E was dosed once daily for 14 days and taxol was dosed as a single administration on day 1.

FIG. 24 shows tumor volume of nude mice bearing human Du145 prostate tumors after treatment by oral administration with Compound F at 20 mg/kg, 40 mg/kg or 80 mg/kg alone, taxol (TXL, 60 mg/kg, i.v.) alone, or the two agents in combination in vivo. Compound F was dosed once daily for 14 days and taxol was dosed as a single administration on day 1.

FIG. 25 shows percentage of body weight change of nude mice bearing human Du145 prostate tumors after treatment by oral administration with Compound F at 20 mg/kg, 40 mg/kg or 80 mg/kg alone, taxol (TXL, 60 mg/kg, i.v.) alone, or the two agents in combination in vivo (in FIG. 24). Compound F was dosed once daily for 14 days and taxol was dosed as a single administration on day 1.

FIG. 26 shows tumor volume of nude mice bearing human Du145 prostate tumors after treatment by oral administration with Compound G or Compound H alone, taxol (TXL, 60 mg/kg, i.v.) alone, or the two agents in combination in vivo. (A) and (C) shows the combination study of Compound G. (B) and (D) shows the combination study of Compound H. Compound H or G was dosed once daily for 14 days and taxol was dosed as a single administration on day 1.

FIG. 27 shows percentage of body weight change of nude mice bearing human Du145 prostate tumors after treatment by oral administration with Compound G or Compound H alone, taxol (TXL, 60 mg/kg, i.v.) alone, or the two agents in combination in vivo. (A) and (C) shows the combination study of Compound G. (B) and (D) shows the combination study of Compound H. Compound H or G was dosed once daily for 14 days and taxol was dosed as a single administration on day 1.

FIG. 28 shows the amino acid sequence of human Thrombospondin-1 precursor (accession number P07996).

FIG. 29 shows the potentiation of Compound A on the anti-tumor effect of taxane against H460 (NSCLC) xenografts (tumor volume regression with treatment by oral administration with Compound A (100 mg/kg) alone, taxol (i.v., 60 mg/kg) alone, and the two agents in combination in vivo). Compound A was administered 3 times per week (p.o.) and taxol was administered on day 1 (i.v.).

FIG. 30 shows the tumor regression of H460 (NSCLC) xenografts with combination therapy using Compound A (100 mg/kg) or SAHA (120 mg/kg) with taxol (60 mg/kg). Compound A was administered 3 times per week (p.o.) and taxol was administered on day 1 (i.v.).

FIG. 31 shows the induction of TSP-1 expression over time in cancer cells after treatment with Compound A.

FIG. 32 shows the suppression of VEGF and bFGF expression in DU145 cells in vitro after 24 hour treatment with Compound A.

FIG. 33 shows the suppression of VEGF and bFGF expression in A549 (NSCLC) xenografts after treatment with Compound A (150 mg/kg, p.o., qdx3).

FIG. 34 shows that synergistic regulation for angiogenesis and cytotoxicity participate in the combination efficacy of Compound A and taxane.

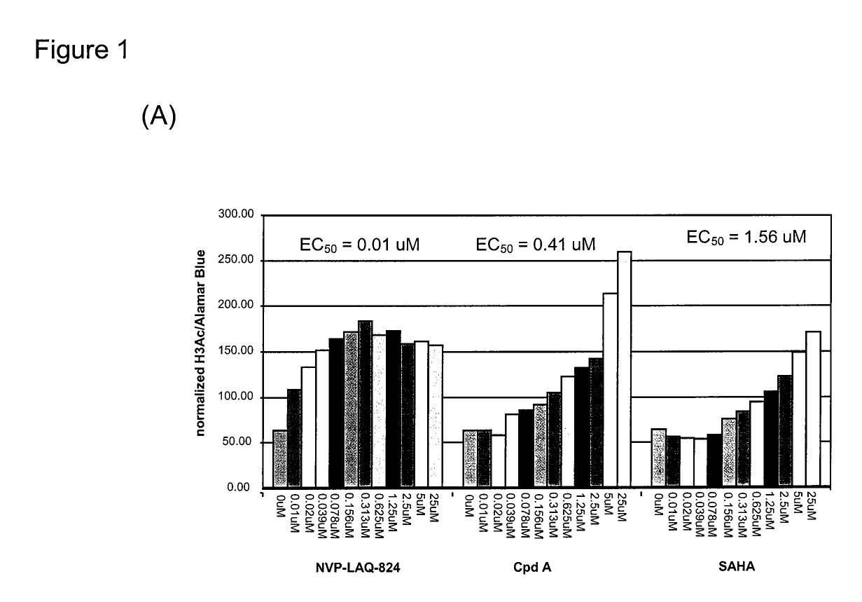

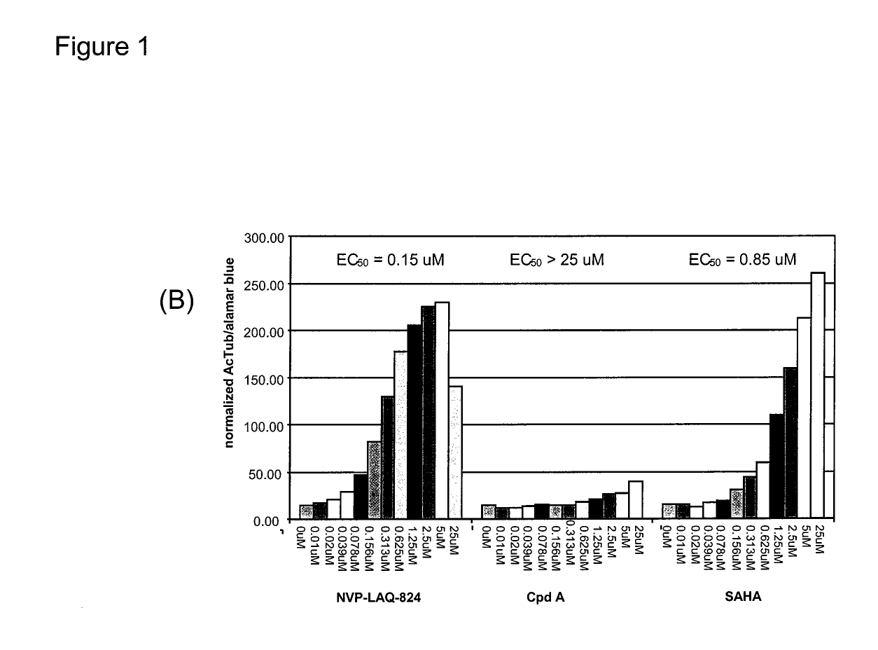

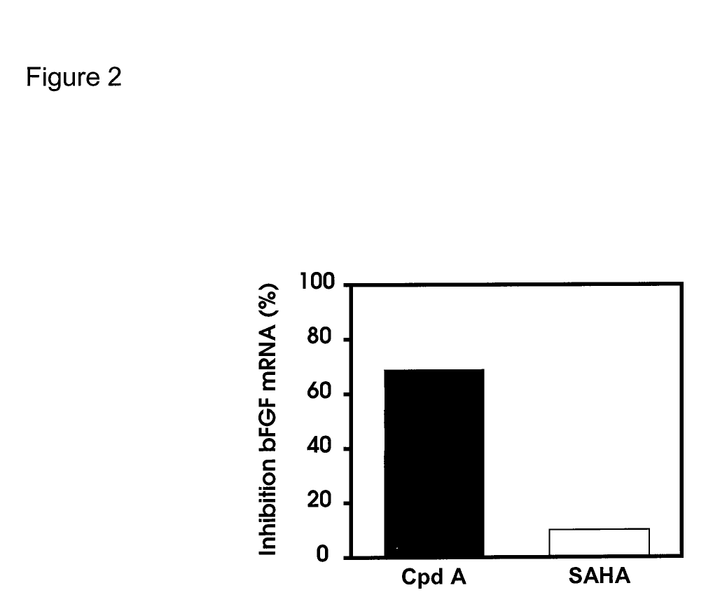

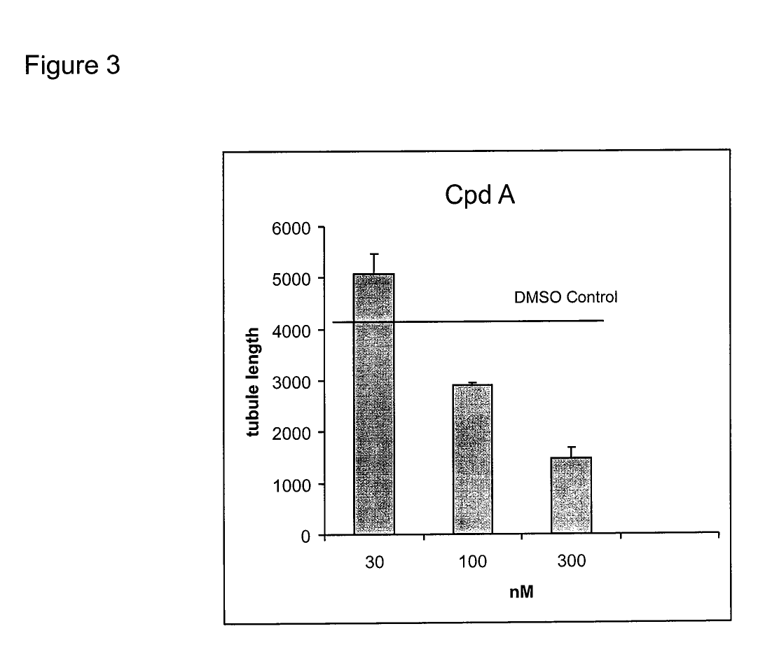

### DETAILED DESCRIPTION OF THE PREFERRED EMBODIMENTS

There is a need to identify pathways which control the sensitivity of cells which exhibit abnormal growth to the treatment with diverse therapeutic agents toward the control of disease manifested by abnormal cell proliferation.

The invention provides a new approach to the therapeutic treatment of disease manifested by abnormal cell growth and/or abnormal cell proliferation. In particular the invention provides a new approach to the therapeutic treatment of cancer. The present inventors have surprisingly discovered that isotype-selective inhibitors of histone deacetylases 1, 2 and/or 3 (HDACs 1-3), as well as isotype-selective inhibitors of HDAC1 and/or HDAC2, potentiate activity of microtubule-stabilization agents, such as taxane compounds. HDAC inhibitors have been shown to possess a broad utility both in vitro and in vivo against many diseases and disorders. See, e.g., Pan, L, et al., HDAC Inhibitors: A Potential New Category of Anti-Tumor Agents, Cellular and Mol. Biol., 2007, 4(5), 337-343.

The patent and scientific literature referred to herein establishes knowledge that is available to those with skill in the art. Each issued patent, patent application, and other publication cited herein is hereby incorporated by reference in its entirety. In the case of inconsistencies, the teachings of the present disclosure will prevail.

Compounds

For purposes of the present invention, the following definitions will be used (unless expressly stated otherwise).

Reference to a compound of “formula (I)”, “formula (II)”, etc., herein is understood to include reference to N-oxides, hydrates, solvates, pharmaceutically acceptable salts, prodrugs and complexes thereof, and racemic and scalemic mixtures, diastereomers, enantiomers and tautomers thereof and unless otherwise indicated.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-3-B-, wherein a is 0 or 1. In such instances, when a is 0 the moiety is B- and when a is 1 the moiety is A-B-. Also, a number of moietes disclosed here may exist in multiple tautomeric forms, all of which are intended to be encompassed by any given tautomeric structure.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-hetoaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, ethenyl, ethynyl, propyl, propenyl, propynyl, and cyclopropyl.

The term “aliphatic” is intended to mean both saturated and unsaturated, straight chain or branched aliphatic hydrocarbons. As will be appreciated by one of ordinary skill in the art, “aliphatic” is intended herein to include, but is not limited to, alkyl, alkenyl or alkynyl moieties.

The term “alkyl” is intended to mean a straight chain or branched aliphatic group having from 1 to 12 carbon atoms, preferably 1-8 carbon atoms, and more preferably 1-6 carbon atoms. Other preferred alkyl groups have from 2 to 12 carbon atoms, preferably 2-8 carbon atoms and more preferably 2-6 carbon atoms. Preferred alkyl groups include, without limitation, methyl, ethyl, propyl, isopropyl, butyl, isobutyl, sec-butyl, tert-butyl, pentyl, hexyl and the like. A “C₀” alkyl (as in “C₀-C₃alkyl”) is a covalent bond.

The term “alkenyl” is intended to mean an unsaturated straight chain or branched aliphatic group with one or more carbon-carbon double bonds, having from 2 to 12 carbon atoms, preferably 2-8 carbon atoms, and more preferably 2-6 carbon atoms. Preferred alkenyl groups include, without limitation, ethenyl, propenyl, butenyl, pentenyl, and hexenyl.

The term “alkynyl” is intended to mean an unsaturated straight chain or branched aliphatic group with one or more carbon-carbon triple bonds, having from 2 to 12 carbon atoms, preferably 2-8 carbon atoms, and more preferably 2-6 carbon atoms. Preferred alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “azolyl” as employed herein is intended to mean a five-membered saturated or unsaturated heterocyclic group containing two or more hetero-atoms as ring atoms, selected from the group consisting of nitrogen, sulfur and oxygen, wherein at least one of the hetero-atoms is a nitrogen atom. Preferred azolyl groups include, but are not limited to, optionally substituted imidazolyl, oxazolyl, thiazolyl, pyrazolyl, isoxazolyl, isothiazolyl, 1,3,4-thiadiazolyl, 1,2,4-thiadiazolyl, 1,2,4-oxadiazolyl, and 1,3,4-oxadiazolyl.

The term “carbocycle” as employed herein is intended to mean a cycloalkyl or aryl moiety. The term “carbocycle” also includes a cycloalkenyl moiety having at least one carbon-carbon double bond.

The term “cycloalkyl” is intended to mean a saturated or unsaturated mono-, bi-, tri- or poly-cyclic hydrocarbon group having about 3 to 15 carbons, preferably having 3 to 12 carbons, preferably 3 to 8 carbons, more preferably 3 to 6 carbons, and more preferably still 5 or 6 carbons. In certain preferred embodiments, the cycloalkyl group is fused to an aryl, heteroaryl or heterocyclic group. Preferred cycloalkyl groups include, without limitation, cyclopenten-2-enone, cyclopenten-2-enol, cyclohex-2-enone, cyclohex-2-enol, cyclopropyl, cyclobutyl, cyclobutenyl, cyclopentyl, cyclopentenyl, cyclohexyl, cyclohexenyl, cycloheptyl, cyclooctyl, etc.

The term “heteroalkyl” is intended to mean a saturated or unsaturated, straight chain or branched aliphatic group, wherein one or more carbon atoms in the group are independently replaced by a moiety selected from the group consisting of O, S, N,N-alkyl, —S(O)—, —S(O)₂—, —S(O)₂NH—, or —NHS(O)₂—.

The term “aryl” is intended to mean a mono-, bi-, tri- or polycyclic aromatic moiety, preferably a C₆-C₁₄aromatic moiety, preferably comprising one to three aromatic rings. Preferably, the aryl group is a C₆-C₁₀aryl group, more preferably a C₆aryl group. Preferred aryl groups include, without limitation, phenyl, naphthyl, anthracenyl, and fluorenyl.

The terms “aralkyl” or “arylalkyl” are intended to mean a group comprising an aryl group covalently linked to an alkyl group. If an aralkyl group is described as “optionally substituted”, it is intended that either or both of the aryl and alkyl moieties may independently be optionally substituted or unsubstituted. Preferably, the aralkyl group is (C₁-C₆)alk(C₆-C₁₀)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The terms “heterocyclyl”, “heterocyclic” or “heterocycle” are intended to mean a group which is a mono-, bi-, or polycyclic structure having from about 3 to about 14 atoms, wherein one or more atoms are independently selected from the group consisting of N, O, and S. The ring structure may be saturated, unsaturated or partially unsaturated. In certain preferred embodiments, the heterocyclic group is non-aromatic, in which case the group is also known as a heterocycloalkyl. In certain preferred embodiments, the heterocyclic group is a bridged heterocyclic group (for example, a bicyclic moiety with a methylene, ethylene or propylene bridge). In a bicyclic or polycyclic structure, one or more rings may be aromatic; for example one ring of a bicyclic heterocycle or one or two rings of a tricyclic heterocycle may be aromatic, as in indan and 9,10-dihydro anthracene. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a heteroaryl group. As used herein, the term “heteroaryl” is intended to mean a mono-, bi-, tri- or polycyclic group having 5 to 18 ring atoms, preferably 5 to 14 ring atoms, more preferably 5, 6, 9, or 10 ring atoms; preferably having 6, 10, or 14 μl electrons shared in a cyclic array; and having, in addition to carbon atoms, between one or more heteroatoms selected from the group consisting of N, O, and S. The term “heteroaryl” is also intended to encompass the N-oxide derivative (or N-oxide derivatives, if the heteroaryl group contains more than one nitrogen such that more than one N-oxide derivative may be formed) of a nitrogen-containing heteroaryl group. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, isoxazolyl, benzo[b]thienyl, naphtha[2,3-b]thianthrenyl, zanthenyl, quinolyl, benzothiazolyl, benzimidazolyl, beta-carbolinyl and perimidinyl. Illustrative examples of N-oxide derivatives of heteroaryl groups include, but are not limited to, pyridyl N-oxide, pyrazinyl N-opxide, pyrimidinyl N-oxide, pyridazinyl N-oxide, triazinyl N-oxide, isoquinolyl N-oxide and quinolyl N-oxide.

The terms “arylene,” “heteroarylene,” or “heterocyclylene” are intended to mean an aryl, heteroaryl, or heterocyclyl group, respectively, as defined hereinabove, that is positioned between and serves to connect two other chemical groups.

A heteroalicyclic group refers specifically to a non-aromatic heterocyclyl radical. A heteroalicyclic may contain unsaturation, but is not aromatic.

A heterocyclylalkyl group refers to a residue in which a heterocyclyl is attached to a parent structure via one of an alkylene, alkylidene, or alkylidyne radical. Examples include (4-methylpiperazin-1-yl)methyl, (morpholin-4-yl)methyl, (pyridine-4-yl)methyl, 2-(oxazolin-2-yl)ethyl, 4-(4-methylpiperazin-1-yl)-2-butenyl, and the like. If a heterocyclylalkyl is described as “optionally substituted” it is meant that both the heterocyclyl and the corresponding alkylene, alkylidene, or alkylidyne radical portion of a heterocyclylalkyl group may be optionally substituted. A “lower heterocyclylalkyl” refers to a heterocyclylalkyl where the “alkyl” portion of the group has one to six carbons.

A heteroalicyclylalkyl group refers specifically to a heterocyclylalkyl where the heterocyclyl portion of the group is non-aromatic.

Preferred heterocyclyls and heteroaryls include, but are not limited to, azepinyl, azetidinyl, acridinyl, azocinyl, benzidolyl, benzimidazolyl, benzofuranyl, benzofurazanyl, benzofuryl, benzothiofuranyl, benzothiophenyl, benzoxazolyl, benzothiazolyl, benzothienyl, benztriazolyl, benztetrazolyl, benzisoxazolyl, benzisothiazolyl, benzimidazolinyl, benzoxazolyl, benzoxadiazolyl, benzopyranyl, carbazolyl, 4aH-carbazolyl, carbolinyl, chromanyl, chromenyl, cinnolinyl, coumarinyl, decahydroquinolinyl, dibenzofuryl, 1,3-dioxolane, 2H,6H-1,5,2-dithiazinyl, dihydrofuro[2,3-b]tetrahydrofuran, dihydroisoindolyl, dihydroquinazolinyl (such as 3,4-dihydro-4-oxo-quinazolinyl), furanyl, furopyridinyl (such as fuor[2,3-c]pyridinyl, furo[3,2-b]pyridinyl or furo[2,3-b]pyridinyl), furyl, furazanyl, hexahydrodiazepinyl, imidazolidinyl, imidazolinyl, imidazolyl, indazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolyl, isoquinolinyl, isothiazolidinyl, isothiazolyl, isoxazolinyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, oxetanyl, 2-oxoazepinyl, 2-oxopiperazinyl, 2-oxopiperidinyl, 2-oxopyrrolodinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolopyridyl, 2H-pyrrolyl, pyrrolyl, quinazolinyl, quinolyl, quinolinyl, 4H-quinolizinyl, quinoxalinyl, quinuclidinyl, tetrahydro-1,1-dioxothienyl, tetrahydrofuranyl, tetrahydrofuryl, tetrahydroisoquinolinyl, tetrahydroquinolinyl, tetrahydropyranyl, tetrazolyl, thiazolidinyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thiamorpholinyl, thiamorpholinyl sulfoxide, thiamorpholuiyl sulfone, thianthrenyl, thiazolyl, thienyl, thienothiazolyl, thienooxazolyl, thienoimidazolyl, thiophenyl, triazinyl, triazinylazepinyl, triazolyl (e.g., 1,2,3-triazolyl, 1,2,4-triazolyl, 1,2,5-triazolyl, 1,3,4-triazolyl), and xanthenyl.

A “halohydrocarbyl” as employed herein is a hydrocarbyl moiety, in which from one to all hydrogens have been replaced with an independently selected halo.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, preferably from one to three, more preferably one or two, independently selected non-hydrogen substituents. Suitable substituents include, without limitation, halo, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, hydroxy, cyano, oxo, carboxy, formyl, nitro, amino, amidino, guanidino,
(b) C₁-C₅alkyl or alkenyl or arylalkyl imino, carbamoyl, azido, carboxamido, mercapto, hydroxy, hydroxyalkyl, alkylaryl, arylalkyl, C₁-C₈alkyl, C₁-C₈alkenyl, C₁-C₈alkoxy, C₁-C₈alkyamino, C₁-C₈alkoxycarbonyl, aryloxycarbonyl, C₂-C₈acyl, —C(O)—N(R³⁰)-alkyl-cycloalkyl, —C(O)—N(R³⁰)-alkyl-heterocyclyl, —C(O)—N(R³⁰)-alkyl-aryl, —C(O)—N(R³⁰)-alkyl-heteroaryl, —C(O)-cycloalkyl, —C(O)-heterocyclyl, —C(O)-aryl, —C(O)-heteroaryl, C₂-C₈acylamino, C₁-C₈alkylthio, arylalkylthio, arylthio, C₁-C₈alkylsulfinyl, arylalkylsulfinyl, arylsulfinyl, C₁-C₈alkylsulfonyl, arylalkylsulfonyl, arylsulfonyl, C₀-C₆N-alkyl carbamoyl, C₂-C₁₅N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇heterocycle, C₅-C₁₅heteroaryl or any of these rings fused or spiro-fused to a cycloalkyl, heterocyclyl, or aryl, wherein each of the foregoing is further optionally substituted with one more moieties listed in (a), above; and
(c) —(CR³²R³³)ₛ—NR³⁰R³¹, wherein s is from 0 (in which case the nitrogen is directly bonded to the moiety that is substituted) to 6, R³² and R³³ are each independently hydrogen, halo, hydroxyl or C₁-C₄alkyl, and R³⁰ and R³¹ are each independently hydrogen, cyano, oxo, hydroxyl, C₁-C₈alkyl, C₁-C₈heteroalkyl, C₁-C₈alkenyl, carboxamido, C₁-C₃alkyl-carboxamido, carboxamido-C₁-C₃alkyl, amidino, C₂-C₈hydroxyalkyl, C₁-C₃alkylaryl, aryl-C₁-C₃alkyl, C₁-C₃alkylheteroaryl, heteroaryl-C₁-C₃alkyl, C₁-C₃alkylheterocyclyl, heterocyclyl-C₁-C₃alkyl C₁-C₃alkylcycloalkyl, cycloalkyl-C₁-C₃alkyl, C₂-C₈alkoxy, C₂-C₈alkoxy-C₁-C₄alkyl, C₁-C₈alkoxycarbonyl, aryloxycarbonyl, aryl-C₁-C₃alkoxycarbonyl, heteroaryloxycarbonyl, heteroaryl-C₁-C₃alkoxycarbonyl, C₁-C₈acyl, C₀-C₈alkyl-carbonyl, aryl-C₀-C₈alkyl-carbonyl, heteroaryl-C₀-C₈alkyl-carbonyl, cycloalkyl-C₀-C₈alkyl-carbonyl, heterocyclyl-C₀-C₈alkyl-carbonyl, C₀-C₈alkyl-NH-carbonyl, aryl-C₀-C₈alkyl-NH-carbonyl, heteroaryl-C₀-C₈alkyl-NH-carbonyl, cycloalkyl-C₀-C₈alkyl-NH-carbonyl, heterocylclyl-C₀-C₈alkyl-NH-carbonyl, cycloalkyl-S(O)₂—, heterocyclyl-S(O)₂—, aryl-S(O)₂—, heteroaryl-S(O)₂—, C₀-C₈alkyl-O-carbonyl, aryl-C₀-C₈alkyl-O-carbonyl, heteroaryl-C₀-C₈alkyl-O-carbonyl, cycloalkyl-C₀-C₈alkyl-O-carbonyl, heterocyclyl-C₀-C₈alkyl-O-carbonyl, C₁-C₈alkylsulfonyl, arylalkylsulfonyl, arylsulfonyl, heteroarylalkylsulfonyl, heteroarylsulfonyl, C₁-C₈alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, or a protecting group, wherein each of the foregoing is further optionally substituted with one more moieties listed in (a), above; or
R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰-Y³¹-), wherein said heterocyclyl may also be bridged (forming a bicyclic moiety with a methylene, ethylene or propylene bridge); wherein
X³⁰ is selected from the group consisting of H, C₁-C₈alkyl, C₂-C₈alkenyl-, C₂-C₈alkynyl-, -C₀-C₃alkyl-C₂-C₈alkenyl-C₀-C₃alkyl, C₀-C₃alkyl-C₂-C₈alkynyl-C₀-C₃alkyl, C₀-C₃alkyl-O—C₀-C₃alkyl-, HO—C₀-C₃alkyl-, C₀-C₄alkyl-N(R³⁰)—C₀-C₃alkyl-, N(R³⁰)(R³¹)-C₀-C₃alkyl-, N(R³⁰)(R³¹)—C₀-C₃alkenyl-, N(R³⁰)(R³¹)—C₀-C₃alkynyl-, (N(R³⁰)(R³¹))₂—C═N—, C₀-C₃alkyl-S(O)₀₋₂—C₀-C₃alkyl-, CF₃—C₀-C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)—heterocyclyl-C₁-C₃alkyl-, wherein the aryl, cycloalkyl, heteroaryl and heterocycyl are optionally substituted with from 1 to 3 substituents from (a); and
Y³¹ is selected from the group consisting of a direct bond, —O—, —N(R³⁰)—, —C(O)—, —O—C(O)—, —C(O)—O—, —N(R³⁰)—C(O)—, —C(O)—N(R³⁰)—, —N(R³⁰)—C(S)—, —C(S)—N(R³⁰)—, —N(R³⁰)—C(O)_N(R³¹)—, —N(R³⁰)—C(NR³⁰)—N(R³¹)—, —N(R³⁰)—C(NR³¹)—, —C(NR³¹)—N(R³⁰)—, —N(R³⁰)—C(S)—N(R³¹)—, —N(R³⁰)—C(O)—O—, —O—C(O)—N(R³¹)—-N(R³⁰)—C(S)—O—, —O—C(S)—N(R³¹)—, —S(O)₀₋₂—, —SO₂N(R³¹)—, —N(R³¹)—SO₂— and —N(R³⁰)—SO₂N(R³¹)—.

A moiety that is substituted is one in which one or more (preferably one to four, preferably from one to three and more preferably one or two), hydrogens have been independently replaced with another chemical substituent. As a non-limiting example, substituted phenyls include 2-fluorophenyl, 3,4-dichlorophenyl, 3-chloro-4-fluoro-phenyl, 2-fluoro-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl —CO—.

When there are two optional substituents bonded to adjacent atoms of a ring structure, such as for example a phenyl, thiophenyl, or pyridinyl, the substituents, together with the atoms to which they are bonded, optionally form a 5- or 6-membered cycloalkyl or heterocycle having 1, 2, or 3 annular heteroatoms.

In a preferred embodiment, a group, such as a hydrocarbyl, heteroalkyl, heterocyclic and/or aryl group is unsubstituted.

In other preferred embodiments, a group, such as a hydrocarbyl, heteroalkyl, heterocyclic and/or aryl group is substituted with from 1 to 4 (preferably from one to three, and more preferably one or two) independently selected substituents.

Preferred substituents on alkyl groups include, but are not limited to, hydroxyl, halogen (e.g., a single halogen substituent or multiple halo substituents; in the latter case, groups such as —CF₃ or an alkyl group bearing Cl₃), oxo, cyano, nitro, alkyl, cycloalkyl, alkenyl, cycloalkenyl, alkynyl, heterocycle, aryl, —OR^{a}, —SR^{a}, —S(═O)R^{e}, —S(═O)₂R^{e}, —P(═O)₂R^{e}, —S(═O)₂OR^{e}, —P(═O)₂OR^{e}, —NR^{b}R^{c}, —NR^{b}S(═O)₂R^{e}, —NR^{b}P(O)₂R^{e}, —S(═O)₂NR^{b}R^{c}, —P(═O)₂NR^{b}R^{c}, —C(═O)OR^{e}, —C(═O)R^{a}, —C(═O)NR^{b}R^{c}, —OC(═O)R^{e}, —OC(═O)NR^{b}R^{c}, —NR^{b}C(═O)OR^{e}, —NR^{d}C(═O)NR^{b}R^{c}, —NR^{d}S(═O)₂NR^{b}R^{c}, —NR^{d}P(═O)₂NR^{c}R^{c}, —NR^{b}C(═O)R^{a} or —NR^{b}P(═O)₂R^{e}, wherein R^{a} is hydrogen, alkyl, cycloalkyl, alkenyl, cycloalkenyl, alkynyl, heterocycle or aryl; R^{b}, R^{c} and R^{d} are independently hydrogen, alkyl, cycloalkyl, heterocycle or aryl, or said R^{b} and R^{c} together with the N to which they are bonded optionally form a heterocycle; and R^{e} is alkyl, cycloalkyl, alkenyl, cycloalkenyl, alkynyl, heterocycle or aryl. In the aforementioned exemplary substituents, groups such as alkyl, cycloalkyl, alkenyl, alkynyl, cycloalkenyl, heterocycle and aryl can themselves be optionally substituted.

Preferred substituents on alkenyl and alkynyl groups include, but are not limited to, alkyl or substituted alkyl, as well as those groups recited as preferred alkyl substituents.

Preferred substituents on cycloalkyl groups include, but are not limited to, nitro, cyano, alkyl or substituted alkyl, as well as those groups recited about as preferred alkyl substituents. Other preferred substituents include, but are not limited to, spiro-attached or fused cyclic substituents, preferably spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on cycloalkenyl groups include, but are not limited to, nitro, cyano, alkyl or substituted alkyl, as well as those groups recited as preferred alkyl substituents. Other preferred substituents include, but are not limited to, spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on aryl groups include, but are not limited to, nitro, cycloalkyl or substituted cycloalkyl, cycloalkenyl or substituted cycloalkenyl, cyano, alkyl or substituted alkyl, as well as those groups recited above as preferred alkyl substituents. Other preferred substituents include, but are not limited to, fused cyclic groups, especially fused cycloalkyl, fused cycloalkenyl, fused heterocycle, or fused aryl, where the aforementioned cycloalkyl, cylcoalkenyl, heterocycle and aryl substituents can themselves be optionally substituted. Still other preferred substituents on aryl groups (phenyl, as a non-limiting example) include, but are not limited to, haloalkyl and those groups recited as preferred alkyl substituents.

Preferred substituents on heterocylic groups include, but are not limited to, cycloalkyl, substituted cycloalkyl, cycloalkenyl, substituted cycloalkenyl, nitro, oxo (i.e., ═O), cyano, alkyl, substituted alkyl, as well as those groups recited as preferred alkyl substituents. Other preferred substituents on heterocyclic groups include, but are not limited to, spiro-attached or fused cylic substituents at any available point or points of attachment, more preferably spiro-attached cycloalkyl, spiro-attached cycloalkenyl, spiro-attached heterocycle (excluding heteroaryl), fused cycloalkyl, fused cycloakenyl, fused heterocycle and fused aryl, where the aforementioned cycloalkyl, cycloalkenyl, heterocycle and aryl substituents can themselves be optionally substituted.

In certain preferred embodiments, a heterocyclic group is substituted on carbon, nitrogen and/or sulfur at one or more positions. Preferred substituents on carbon include those groups recited as preferred alkyl substituents. Preferred substituents on nitrogen include, but are not limited to alkyl, aryl, aralkyl, alkylcarbonyl, alkylsulfonyl, arylcarbonyl, arylsulfonyl, alkoxycarbonyl, or aralkoxycarbonyl. Preferred substituents on sulfur include, but are not limited to, oxo and C₁₋₆alkyl. In certain preferred embodiments, nitrogen and sulfur heteroatoms may independently be optionally oxidized and nitrogen heteroatoms may independently be optionally quaternized.

Especially preferred substituents on ring groups, such as aryl, heteroaryl, cycloalkyl and heterocyclyl, include halogen, alkoxy and alkyl.

Especially preferred substituents on alkyl groups include halogen and hydroxy.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally optionally substituted. The term “sulfonamido” refers to a sulfonamide substituent attached by either the sulfur or the nitrogen atom. The term “amino” is meant to include NH₂, alkylamino, di-alkyl-amino, arylamino, and cyclic amino groups. The term “ureido” as employed herein refers to a substituted or unsubstituted urea moiety.

The term “radical” as used herein means a chemical moiety comprising one or more unpaired electrons.

Where optional substituents are chosen from “one or more” groups it is to be understood that this definition includes all substituents being chosen from one of the specified groups or the substituents being chosen from two or more of the specified groups.

In addition, substituents on cyclic moieties (i.e., cycloalkyl, heterocyclyl, aryl, heteroaryl) include 5- to 6-membered mono- and 9- to 14-membered bi-cyclic moieties fused to the parent cyclic moiety to form a bi- or tri-cyclic fused ring system. Substituents on cyclic moieties also include 5- to 6-membered mono- and 9- to 14-membered bi-cyclic moieties attached to the parent cyclic moiety by a covalent bond to form a bi- or tri-cyclic bi-ring system. For example, an optionally substituted phenyl includes, but is not limited to, the following:

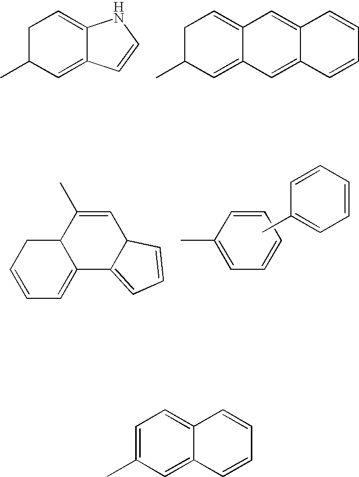

When a carbocyclic or heterocyclic group is substituted by two C₁₋₆ alkyl groups, the two alkyl groups may combine together to form an alkylene chain, preferably a C₁₋₃ alkylene chain. Carbocyclic or heterocyclic groups having this crosslinked structure include bicyclo[2.2.2]octanyl and norbornanyl.

Throughout the specification, preferred embodiments of one or more chemical substituents are identified. Also preferred are combinations of preferred embodiments. For example, the invention describes preferred embodiments of L in the compounds of formula (I) and describes preferred embodiments of group Y. Thus, as an example, also contemplated as within the scope of the invention are compounds in which preferred examples of L are as described and in which preferred examples of group Y are as described.

The term “therapeutically effective amount” as employed herein is an amount of a compound of the invention, that when administered to a patient, elicits the desired therapeutic effect. The therapeutic effect is dependent upon the disease being treated and the results desired. As such, the therapeutic effect can be treatment of a disease-state. The amount of a compound which constitutes a “therapeutically effective amount” will vary depending on the compound, the disease state and its severity, the age of the patient to be treated, and the like. The therapeutically effective amount can be determined routinely by one of ordinary skill in the art.

The term “patient” as employed herein for the purposes of the present invention includes humans and other animals, particularly mammals, and other organisms. Thus the compounds, compositions and methods of the present invention are applicable to both human therapy and veterinary applications. In a preferred embodiment the patient is a mammal, and in a most preferred embodiment the patient is human.

The terms “treating”, “treatment”, or the like, as used herein covers the treatment of a disease-state in an animal and includes at least one of: (i) preventing the disease-state from occurring, in particular, when such animal is predisposed to the disease-state but has not yet been diagnosed as having it; (ii) inhibiting the disease-state, i.e., partially or completely arresting its development; (iii) relieving the disease-state, i.e., causing regression of symptoms of the disease-state, or ameliorating a symptom of the disease; and (iv) reversal or regression of the disease-state, preferably eliminating or curing of the disease. In a preferred embodiment of the present invention the animal is a mammal, preferably a primate, more preferably a human. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art. In a preferred embodiment, treatment includes at least one of (ii), (iii) and (iv).

The foregoing merely summarizes one aspect and embodiments of the invention and is not intended to be limiting in nature. This aspect and embodiments are described more fully below.

Compounds that are particularly useful in the methods according to the invention include histone deacetylase (HDAC) inhibitors that are selective for HDAC 1, HDAC2 and HDAC3. These compounds are shown herein to induce expression of both MT3 and TSP1. Generically, HDAC inhibitors having a structure as described in US 2004/0106599, U.S. Pat. No. 6,897,220, US 2006/0058298, US 2005/0288282, WO 2005/030705, US 2005/0245518, U.S. Ser. No. 11/687,398, U.S. Ser. No. 11/696,8801, U.S. 60/906,733 have been shown to be selective for HDAC1, HDAC2 and/or HDAC3.

Particularly useful compounds selective for HDAC1, HDAC2 and HDAC3 include those having a structure represented by Formula (I):

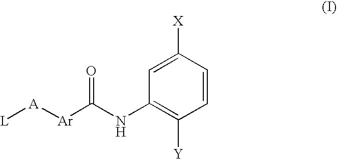

and N-oxides, hydrates, solvates, pharmaceutically acceptable salts, prodrugs and complexes thereof, and racemic and scalemic mixtures, diastereomers, enantiomers and tautomers thereof, wherein
X is H, halo-, C₁-C₄-alkyl, C₁-C₄-alkoxy, —CH₂F, —CHF₂, —CF₃, aryl or heteroaryl, each of which is optionally substituted (preferably with one to three substituents independently selected from halo, —CN, —CH═N(OH), hydroxy, C₁-C₃-hydrocarbyl, —O—C₁-C₄alkyl, methoxy, or mono-, di-, or tri-halo substituted alkyl),
Y is —NH₂ or OH;
Ar is arylene or heteroarylene, each of which is optionally substituted;
A is selected from the group consisting of a covalent bond, M¹-L²-M¹, and L²-M²-L² wherein
   L², at each occurrence, is independently selected from the group consisting of a chemical bond, C₀-C₄ hydrocarbyl, C₀-C₄-hydrocarbyl-(NH)—C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-SO—C₀-C₄-hydrocarbyl, C₀-C₄-hydrocarbyl-SO₂-C₀-C₄-hydrocarbyl, C₀-C₄-hydrocarbyl-NH—CO—C₀-C₄-hydrocarbyl, and C₀-C₄-hydrocarbyl-CO—NH-C₀-C₄-hydrocarbyl, provided that L² is not a chemical bond when X¹ is M¹-L²-M¹;
   M¹, at each occurrence, is independently selected from the group consisting of —O—, —N(R⁷)—, —S—, —S(O)—, S(O)₂—, —S(O)₂N(R⁷)—, —N(R⁷)—S(O)₂—, —C(O)—, —C(O)—NH—, —NH—C(O)—, —NH—C(O)—O— and —O—C(O)—NH—, wherein R⁷ is selected from the group consisting of hydrogen, alkyl, aryl, aralkyl, acyl, heterocyclyl, and heteroaryl; and
   M² is selected from the group consisting of M¹, heteroarylene, and heterocyclylene, either of which rings optionally is substituted; and
L is selected from the group consisting of H, cycloalkyl, aryl, heteroaryl, or heterocyclyl, each of which is optionally substituted and each of which is optionally fused to one or more aryl or heteroaryl rings, or to one or more saturated or partially unsaturated cycloalkyl or heterocyclic rings, each of which rings is optionally substituted.
   In other compounds, X is phenyl, thienyl, furanyl, pyridyl, or pyrimidyl.
   In other compounds of formula (I), Y is —NH₂.
   In other compounds of formula (I), Ar is phenyl, preferably unsubstituted phenyl.
   In other compounds of formula (I), A is —N(R⁷)—(CH₂)—.
   In other compounds of formula (I), L is optionally substituted -heteroaryl-heteroaryl, optionally substituted -alkyl or optionally substituted heteroaryl.
   In other compounds of formula (I), R⁷ is H.

Other compounds of Formula (I) include those having a structure represented by Formula (II):

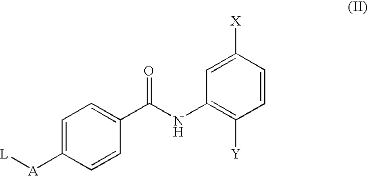

and N-oxides, hydrates, solvates, pharmaceutically acceptable salts, prodrugs and complexes thereof, and racemic and scalemic mixtures, diastereomers, enantiomers and tautomers thereof, wherein
X is H, phenyl, thienyl, furanyl, pyridyl or pyrimidyl, each of which is optionally substituted;
Y is —NH₂;
A is —N(R⁷)—(CH₂)—; and
L is -heteroaryl-heteroaryl, -alkyl or heteroaryl, each of which is optionally substituted;
   wherein R⁷ is selected from the group consisting of hydrogen, alkyl, aryl, aralkyl, acyl, heterocyclyl, and heteroaryl.
   In other compounds of formula (II), X is H.
   In other compounds of formula (II), X is phenyl or pyridyl, each of which is optionally substituted.
   In other compounds of formula (II), L is optionally substituted heteroaryl-heteroaryl.
   In other compounds of formula (II), R⁷ is H.

Other particularly useful compounds selective for HDAC1, HDAC2 and HDAC3 include those having a structure represented by Formula (III):

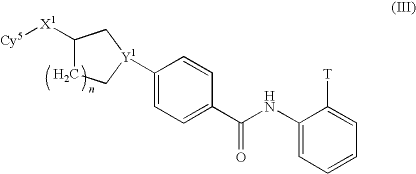

and N-oxides, hydrates, solvates, pharmaceutically acceptable salts, prodrugs and complexes thereof, and racemic and scalemic mixtures, diastereomers, enantiomers and tautomers thereof, wherein
Cy⁵ is aryl, or heteroaryl, each of which is optionally substituted and wherein each of aryl and heteroaryl is optionally fused to one or more aryl or heteroaryl rings, or to one or more saturated or partially unsaturated cycloalkyl or heterocyclic rings, each of which rings is optionally substituted;
X¹ is selected from the group consisting of: a covalent bond, C₀-C₄-hydrocarbyl, C₀-C₄-hydrocarbyl-(CO)—C₀-C₄-hydrocarbyl, C₀-C₄-hydrocarbyl-N(R⁸)—C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-(SO)—C₀-C₄-hydrocarbyl, C₀-C₄-hydrocarbyl-(SO₂)—C₀-C₄-hydrocarbyl, C₀-C₄-hydrocarbyl-(NH)—(CO)—C₀-C₄-hydrocarbyl, C₀-C₄-hydrocarbyl-(CO)—(NH)-C₀-C₄-hydrocarbyl, —NH—CO—NH—, —NH—CS—NH—, —O—CO—O—, —O—CS—O—, —NH—C(NH)—NH—, —S(O)₂—N(R⁸)—, —N(R⁸)—S(O)₂—, —NH—C(O)—O—, and —O—C(O)—NH—;
wherein R⁸ is selected from the group consisting of hydrogen, C₁-C₅-alkyl, aryl, aralkyl, acyl, heterocyclyl, heteroaryl, SO₂-alkyl, SO₂-aryl, CO-alkyl, CO-aryl, CO—NH-alkyl, CO—NH-aryl, CO—O-alkyl and CO—O-aryl, each of which is optionally substituted;
n is 0 to 4;
Y¹ is N or CH; and
T is NH₂ or OH.
   In other compounds of formula (III), T is —NH₂.
   In other compounds of formula (III), Y¹ is N.
   In other compounds of formula (III), n is 1.
   In other compounds of formula (III), X¹ is —N(H)—.
   In other compounds of formula (III), Cy5 is optionally substituted heteroaryl.

Particularly useful compounds selective for HDAC1 and HDAC2 include those having a structure represented by formula (IV):

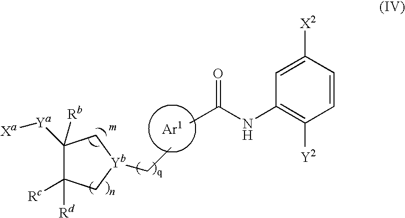

and N-oxides, hydrates, solvates, pharmaceutically acceptable salts, prodrugs and complexes thereof, and racemic and scalemic mixtures, diastereomers, enantiomers and tautomers thereof, wherein
X² is aryl, cycloalkyl, heteroaryl or heterocyclyl, each of which is optionally substituted;
Ar¹ is aryl, heteroaryl, cycloalkyl or heterocyclyl, each of which is optionally substituted;
R^{a} is H or an optional substituent, preferably halo;
R^{b}, R^{c} and R^{d} are each independently hydrogen, C₁-C₈ alkyl, aryl, heteroaryl, cycloalkyl, heterocyclyl or halo; or
R^{b} and R^{c} together with the atoms to which they are bonded, optionally form a 5- or 6-membered cycloalkyl or heterocycloalkyl having 1 or 2 annular heteroatoms; each of which is optionally substituted with from 1 to 3 substituents;
Y² is —NH₂ or —OH;
Y^{b} is —N— or —CH—;
Y^{a} is a direct bond, —O—, —N(R³⁴)—, —C(O)—, —OC(O)—, —C(O)O—, —N(R³⁴)—C(O)—, —C(O)—N(R³⁴)—, —N(R³⁴) —C(S)—, —C(S)—N(R³⁴)—, —N(R³⁴)—C(O)—N(R³⁵)—, —N(R³⁴)—C(NR³⁴)—N(R³⁵)—, —N(R³⁴)—C(NR³⁵)—, —C(NR³⁵)—N(R³⁴)—, —N(R³⁴)—C(S)—N(R³⁵)—, —N(R³⁴)—C(O)—O—, —O—C(O)—N(R³⁴)—, —N(R³⁴)—C(S)O—, —O—C(S)—N(R³⁵)—, —S(O)₀₋₂—, —SO₂N(R³⁵)—, —N(R³⁵)—SO₂—, N(R³⁴)—S(O)₂, N(R³⁵)—, —O—C₁-C₃alkyl-, —N(R³⁴)—C₁-C₃alkyl-, —C(O)—C₁-C₃alkyl- or —O—C(O)—C₁-C₃alkyl-;
X³ is C₁-C₈alkyl-, C₁-C₈alkenyl-, C₁-C₈alkynyl-, C₀-C₃alkyl-C₁-C₈alkenyl-C₀-C₃alkyl-, C₀-C₃alkyl-C₁-C₈alkynyl-C₀-C₃alkyl-, C₁-C₃alkyl-O—C₁-C₃alkyl-, HO—C₁-C₃alkyl-, C₁-C₄alkyl-N(R³⁴)—C₀-C₃alkyl-, N(R³⁴)(R³⁵)—C₀-C₃alkyl-, C₁-C₃alkyl-S(O)₀₋₂—C₁-C₃alkyl-, CF₃-C₀-C₃alkyl-, CF₂H-C₀-C₃alkyl-, C₁-C₈heteroalkyl-, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, aryl-C₀-C₂alkyl-heterocyclyl-C₀-C₂alkyl-, heteroaryl-C₀-C₂alkyl-heterocyclyl-C₀-C₂alkyl-, N(R³⁴)(R³⁵)-heterocyclyl-C₀-C₃alkyl-, heteroaryl-C₀-C₃alkyl-heterocyclyl- or C₁-C₄alkyl-CH(N(R³⁴)(R³⁵))—C(O)—N(R³⁴)-aryl-, wherein the aryl, cycloalkyl, heteroaryl and heterocycyl are optionally substituted with from 1 to 3 independently selected substituents;
   or
X^{a}-Y^{a}- is selected from the group consisting of H—, halo-, HO—, HS—, HC(O)—, HOC(O)—, C₁-C₄alkyl-, H₂N—, (R³⁴)(R³⁵)N—, C₁-C₄alkyl-NH—, (C₁-C₄alkyl)₂-N—, HC(O)N(R³⁴)—, (R³⁴)(R³⁵)N—S(O)₂—(R³⁶)—, (R³⁴)(R³⁵)N—C(O)—, H₂N—C(O)—, HC(S)N(R³⁴)—, (R³⁴)(R³⁵)N—C(S)—, H₂N—C(S)—, (R³⁴)(R³⁵)N—C(O)—O—, (R³⁴)(R³⁵)N—C(S)—O—, (R³⁴)(R³⁵)N—C(O)—N(R³⁶)—, (C₁-C₃alkylN)₂—C═N—, (R³⁴)(R³⁵)N—C(NR³⁷)—N(R³⁶)—, (R³⁴)(R³⁵)N—C(NR³⁶)—, cycloalkyl-C₀-C₂alkyl-C(NR³⁶)—, heterocyclyl-C₀-C₂alkyl-C(NR³⁶)-, aryl-C₀-C₂alkyl-C(NR³⁶)—, heteroaryl-C₀-C₂alkyl-C(NR³⁶)—, C₀-C₃alkyl-C(NR³⁶)—, C₁-C₄alkyl-S(O)₂—N(R³⁶)—, CF₃-C₀-C₄alkyl-S(O)₂—N(R³⁶)—, CF₃-C₀-C₄alkyl-C(O)—N(R³⁶)—, aryl-C₀-C₄alkyl-S(O)₂—N(R³⁶)—, heteroaryl-C₀-C₄alkyl-S(O)₂—N(R³⁶)—, cycloalkyl-C₀-C₄alkyl-S(O)₂—N(R³⁶)—, heterocyclyl-C₀-C₄alkyl-S(O)₂—N(R³⁶)—, C₁-C₄alkyl-O—C(O)—NH—, C₁-C₄alkyl-O—C(O)—N(H)—C₁-C₄alkyl-, C₁-C₄alkyl-N(H)—C(O)—N(H)—, C₁-C₄alkyl-NH—C(O)—O—, C₁-C₄alkyl-C(O)—N(H)—, C₁-C₄alkyl-O—C(S)—N(H)—, C₁-C₄alkyl-N(H)—C(S)—N(H)—, C₁-C₄alkyl-N(H)—C(S)—O—, C₁-C₄alkyl-C(S)—N(H)—, Me-C(O)—O—, Me-C(O)—N(H)—, aryl-C₀-C₄alkyl-O—C(O)—N(H)—, aryl-C₀-C₄alkyl-O—C(O)—N(C₁-C₄alkyl)-, aryl-C₀-C₄alkyl-C(O)—N(H)—, heteroaryl-C₀-C₄alkyl-O—C(O)—N(H)—, heteroaryl-C₀-C₄alkyl-O—C(O)—N(C₁-C₄alkyl)-, heteroaryl-C₀-C₄alkyl-C(O)—N(H)—, aryl-C₀-C₄alkyl-N(H)—C(O)—O—, heteroaryl-C₀-C₄alkyl-N(H)—C(O)—O—, heterocyclyl-C₀-C₄alkyl-O—C(O)—N(H)—, heterocyclyl-C₀-C₄alkyl-O—C(O)—N(C₁-C₄alkyl)-, heterocyclyl-C₀-C₄alkyl-C(O)—N(H)—, cycloalkyl-C₀-C₄alkyl-O—C(O)—N(H)—, cycloalkyl-C₀-C₄alkyl-O—C(O)—N(C₁-C₄alkyl)-, cycloalkyl-C₀-C₄alkyl-C(O)—N(H)—, heterocyclyl-C₀-C₄alkyl-N(H)—C(O)—O—, cycloalkyl-C₀-C₄alkyl-N(H)—C(O)—O—, heterocyclyl-C₀-C₄alkyl-C(O)—N(H)—, aryl-C₀-C₄alkyl-N(H)—C(O)—N(H)—, aryl-C₀-C₄alkyl-N(H)—, aryl-C₀-C₄alkyl-O—, aryl-C₀-C₄alkyl-S(O)₀₋₂—, heteroaryl-C₀-C₄alkyl-N(H)—C(O)—N(H)—, heteroaryl-C₀-C₄alkyl-N(H)—, heteroaryl-C₀-C₄alkyl-O—, heteroaryl-C₀-C₄alkyl-S(O)₀₋₂—, heterocyclyl-C₀-C₄alkyl-N(H)—C(O)—N(H)—, heterocyclyl-C₀-C₄alkyl-N(H)—, heterocyclyl-C₀-C₄alkyl-O—, heterocyclyl-C₀-C₄alkyl-S(O)₀₋₂—, cycloalkyl-C₀-C₄alkyl-N(H)—C(O)—N(H)—, cycloalkyl-C₀-C₄alkyl-N(H)—, cycloalkyl-C₀-C₄alkyl-O—, cycloalkyl-C₀-C₄alkyl-S(O)₀₋₂—, aryl-C₀-C₄alkyl-C(S)—N(H)—, heteroaryl-C₀-C₄alkyl-C(S)—N(H)—, aryl-C₀-C₄alkyl-O—C(S)—N(H)—, heteroaryl-C₀-C₄alkyl-O—C(S)—N(H)—, aryl-C₀-C₄alkyl-N(H)—C(S)—O—, heteroaryl-C₀-C₄alkyl-N(H)—C(S)—O—, heterocyclyl-C₀-C₄alkyl-C(S)—N(H)—, cycloalkyl-C₀-C₄alkyl-C(S)—N(H)—, heterocyclyl-C₀-C₄alkyl-O—C(S)—N(H)—, cycloalkyl-C₀-C₄alkyl-O—C(S)—N(H)—, heterocyclyl-C₀-C₄alkyl-N(H)—C(S)—O—, cycloalkyl-C₀-C₄alkyl-N(H)—C(S)—O—, heterocyclyl-C₀-C₄alkyl-C(S)—N(H)—, aryl-C₀-C₄alkyl-N(H)—C(S)—NH—, heteroaryl-C₀-C₄alkyl-N(H)—C(S)—N(H)—, heterocyclyl-C₀-C₄alkyl-N(H)—C(S)—N(H)—, cycloalkyl-C₀-C₄alkyl-N(H)—C(S)—N(H)—, C₁-C₄alkyl-O—C₁-C₄alkyl-C(O)—N(H)—, C₁-C₄alkyl-O—C₂-C₄alkyl-O—C(O)—N(H)—, C₁-C₄alkyl-O—C₂-C₄alkyl-N(H)—C(O)—N(H)—, C₁-C₄alkyl-O—C₂-C₄alkyl-N(H)—, C₁-C₄alkyl-O—C₂-C₄alkyl-O—, C₁-C₄alkyl-O—C₂-C₄alkyl-N(H)—C(O)—O—, HO—C₁-C₄alkyl-C(O)—N(H)—, HO—C₁-C₄alkyl-N(H)—, HO—C₁-C₄alkyl-N(R³)—, HO—C₁-C₄alkyl-O—, HO—C₁-C₄alkyl-S(O)₀₋₂—, HO—C₂-C₄alkyl-O—C(O)—N(H)—, HO—C₂-C₄alkyl-N(H)—C(O)—N(H)—, HO—C₂-C₄alkyl-N(H)—C(O)—O—, C₁-C₄alkyl-O—C₁-C₄alkyl-C(S)—N(H)—, C₁-C₄alkyl-O—C₂-C₄alkyl-O—C(S)—N(H)—, C₁-C₄alkyl-O—C₂-C₄alkyl-N(H)C(S)—N(H)—, C₁-C₄alkyl-O—C₂-C₄alkyl-N(H)—C(S)—O—, HO—C₂-C₄alkyl-O—C(S)—N(H)—, HO—C₂-C₄alkyl-N(H)—C(S)—N(H)—, HO—C₂-C₄alkyl-N(H)—C(S)—O—, (C₁-C₄alkyl)₂N—C₁-C₄alkyl-C(O)—N(H)—, (C₀-C₄alkyl)-O—C₁-C₄alkyl-C(O)—N(H)—, (C₀-C₄alkyl)-O—C₁-C₄alkyl-C(S)—N(H)—, (C₀-C₄alkyl)-O—C₁-C₄alkyl-C(O)—O—, (C₀-C₄alkyl)-O—C₂-C₄alkyl-N(H)—C(O)—N(H)—, (C₀-C₄alkyl)-O—C₂-C₄alkyl-O—C(O)—N(H)—, (C₀-C₄alkyl)-O—C₂-C₄alkyl-N(H)—C(NH)—N(H)—, (C₀-C₄alkyl)-O—C₂-C₄alkyl-N(H)—C(O)—, (C₁-C₄alkyl)₂N—C₂-C₄alkyl-O—C(O)—N(H)—, (C₁-C₄alkyl)₂N—C₂-C₄alkyl-N(H)—, (C₁-C₄alkyl)₂N—C₂-C₄alkyl-O—, (C₁-C₄alkyl)₂N—C₂-C₄alkyl-S(O)₀₋₂—, (C₁-C₄alkyl)₂N—C₂-C₄alkyl-N(H)—C(O)—N(H)—, (C₁-C₄alkyl)₂N—C₂-C₄alkyl-N(H)—C(O)—O—, (C₁-C₄alkyl)₂N—C₁-C₄alkyl-C(S)—N(H)—, (C₁-C₄alkyl)₂N—C₂-C₄alkyl-N(H)—C(S)—N(H)—, (C₁-C₄alkyl)₂N—C₂-C₄alkyl-N(H)—C(S)—O—, (C₁-C₄alkyl)-O—C(O)C₁-C₈alkyl-C(O)—(H)—, HO—C(O)C₁-C₈alkyl-C(O)—N(H)—, HO—NH—C(O)C₁-C₈alkyl-C(O)—N(H)—, CF₂H-C₀-C₄alkyl-C(O)—N(H)—, CF₃-C₀-C₄alkyl-C(O)—N(H)—, CF₃-C₀-C₄alkyl-N(H)—, CF₃-C₀-C₄alkyl-N(R³)—, CF₃-C₀-C₄alkyl-O—, CF₃-C₀-C₄alkyl-S(O)₀₋₂—, CF₃-C₀-C₄alkyl-O—C(O)—N(H)—, CF₃-C₀-C₄alkyl-N(H)C(O)—N(H)—, CF₃-C₀-C₄alkyl-N(H)—C(O)—O—, CF₃-C₀-C₄alkyl-O—C(S)—N(H)—, CF₃-C₀-C₄alkyl-N(H)—C(S)—N(H)—, CF₃-C₀-C₄alkyl-N(H)—C(S)—O—, CF₃-C₀-C₄alkyl-C(S)—N(H)—, CF₂H-C₀-C₄alkyl-N(H)—, CF₂H-C₀-C₄alkyl-O—, CF₂H-C₀-C₄alkyl-S(O)₀₋₂—, CF₂H-C₀-C₄alkyl-O—C(O)—N(H)—, CF₂H-C₀-C₄alkyl-N(H)C(O)—N(H)—, CF₂H-C₀-C₄alkyl-N(H)—C(O)—O—, CF₂H-C₀-C₄alkyl-O—C(S)—N(H)—, CF₂H-C₀-C₄alkyl-N(H)—C(S)—N(H)—, CF₂H-C₀-C₄alkyl-N(H)—C(S)—O—, CF₂H-C₀-C₄alkyl-C(S)—N(H)—, (H)(R³⁴)N—C₁-C₃alkyl-, (H)(R³⁴)N—C₁-C₃alkyl-, HO—C₁-C₃alkyl-, (H)(R³⁴)N—S(O)₂—N(R³⁵)—, (H)(R³⁵)N—S(O)₂—, (H)(R³⁴)N—C(S)—O—, (H)(R³⁴)N—C(O)—O—, (H)(R³⁴)N—C(S)—N(R³⁵)—, (H)(R³⁴)N—C(NR³⁵)—, (H)(R³⁴)N—C(NR³⁴)—N(R³⁸)—, (H)(R³⁴)N—C(O)—N(R³⁵)—, HO—C(O)—C₁-C₃alkyl-, C₁-C₄alkyl-S(O)₂—NH— and ((R³⁴)(R³⁵)N)₂—C═N—;
m and n are independently 0, 1, 2 or 3;
q is 0, 1 or 2; and
R³⁴, R³⁵, R³⁶ and R³⁷ are each independently selected rom the group consisting of hydrogen, cyano, oxo, hydroxyl, —C₁-C₈alkyl, C₁-C₈heteroalkyl, C₁-C₈alkenyl, carboxamido, C₁-C₃alkyl-carboxamido-, carboxamido-C₁-C₃alkyl-, amidino, C₂-C₈hydroxyalkyl, C₁-C₃alkylaryl-, aryl-C₁-C₃alkyl-, C₁-C₃alkylheteroaryl-, heteroaryl-C₁-C₃alkyl-, C₁-C₃alkylheterocyclyl-, heterocyclyl-C₁-C₃alkyl-, C₁-C₃alkylcycloalkyl-, cycloalkyl-C₁-C₃alkyl-, C₂-C₈alkoxy-, C₂-C₈alkoxy-C₁-C₄alkyl-, C₁-C₈alkoxycarbonyl-, aryloxycarbonyl-, aryl-C₁-C₃alkoxycarbonyl-, heteroaryloxycarbonyl-, heteroaryl-C₁-C₃alkoxycarbonyl-, C₁-C₈acyl, C₀-C₈alkyl-carbonyl-, aryl-C₀-C₈alkyl-carbonyl-, heteroaryl-C₀-C₈alkyl-carbonyl-, cycloalkyl-C₀-C₈alkyl-carbonyl-, C₀-C₈alkyl-N(H)-carbonyl-, aryl-C₀-C₈alkyl-N(H)-carbonyl-, heteroaryl-C₀-C₈alkyl-N(H)-carbonyl-, cycloalkyl-C₀-C₈alkyl-N(H)-carbonyl-, C₀-C₈alkyl-O-carbonyl-, aryl-C₀-C₈alkyl-O-carbonyl-, heteroaryl-C₀-C₈alkyl-O-carbonyl-, cycloalkyl-C₀-C₈alkyl-O-carbonyl-, C₁-C₈ alkylsulfonyl-, arylalkylsulfonyl-, arylsulfonyl-, heteroarylalkylsulfonyl-, heteroarylsulfonyl-, C₁-C₈alkyl-N(H)-sulfonyl-, arylalkyl-N(H)-sulfonyl-, aryl-N(H)-sulfonyl-, heteroarylalkyl-N(H)-sulfonyl-, heteroaryl-N(H)-sulfonyl, aroyl, aryl, cycloalkyl, heterocyclyl, heteroaryl, aryl-C₁-C₃alkyl-, cycloalkyl-C₁-C₃alkyl-, heterocyclyl-C₁-C₃ alkyl-, heteroaryl-C₁-C₃ alkyl-, and a protecting group, wherein each of the foregoing is further optionally substituted with one more moieties; or
R³⁴ and R³⁵ taken together with the N to which they are attached form a heterocyclyl or heteroaryl, each of which is optionally substituted with from 1 to 3 substituents, wherein the heterocyclyl may also be bridged (forming a bicyclic moiety with a methylene, ethylene or propylene bridge),
provided that 1) when Y^{b} is N, then m is not 0 if Y^{a} is bound to the ring comprising Y, via a N, S or O in Y^{a}, or 2) when m and n are both 0 then Y^{b} is —CH—.
   In other compounds of formula (IV), X² is aryl, preferably phenyl.
   In other compounds of formula (IV), X² is heteroaryl, preferably pyridyl.
   In other compounds of formula (IV), Y² is —NH₂.
   In other compounds of formula (IV), Ar¹ is optionally substituted phenyl.
   In other compounds of formula (IV), n and m are each 1.
   In other compounds of formula (IV), each of R^{b}, R^{c} and R^{d} are H.
   In other compounds of formula (IV), -Y^{a}-X^{a} is —N(R³⁴)(R³⁵).

Other compounds of formula (IV) have the formula (IVa):

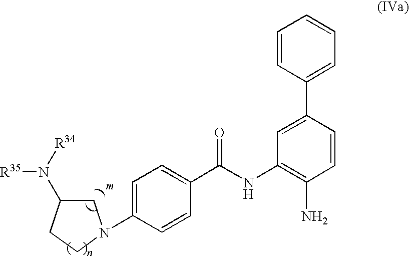

wherein m, n, R³⁴ and R³⁵ are as defined for formula (IV).

Other particularly useful compounds selective for HDAC1 and HDAC2 include those having a structure represented by formula (V):

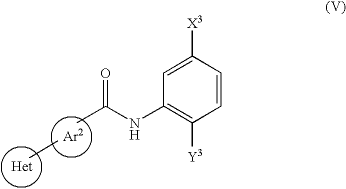

and N-oxides, hydrates, solvates, pharmaceutically acceptable salts, prodrugs or complexes thereof, and racemic and scalemic mixtures, diastereomers and enantiomers thereof wherein
X³ is aryl, cycloalkyl, heteroaryl or heterocyclyl, each of which is optionally substituted;
Y³ is —NH₂ or —OH;
Ar² is optionally substituted aryl or optionally substituted heteroaryl; and
Het is an optionally substituted heterocyclyl.
   In other compounds according to formula (V), X³ is heteroaryl, preferably pyridyl.
   In other compound according to formula (V), X³ is aryl, preferably phenyl.
   In other compounds according to formula (V), Y³ is —NH₂.
   In other compounds according to formula (V), Ar² is aryl, preferably phenyl.
   In other compounds according to formula (V), Het is an optionally substituted six-membered heterocyclyl.
   In other compounds according to formula (V), Het is optionally substituted piperazinyl.
   In other compounds according to formula (V), Het is piperazinyl optionally substituted with alkyl.

These and other compounds that can be readily identified by the methods taught herein are useful in the methods according to the invention. Particularly interesting HDAC inhibitors useful in the invention include those having the structures shown in Table 1

TABLE 1
structure and IC50's of compound A, B, C, D, E, F, G and H in vitro against recombinant human HDAC enzymes

| | | IC50 (μM) | | | | | | | |
|---|---|---|---|---|---|---|---|---|---|
| STRUCTURE | NAME | HD1 | HD2 | HD3 | HD4 | HD5 | HD6 | HD7 | HD8 |
| 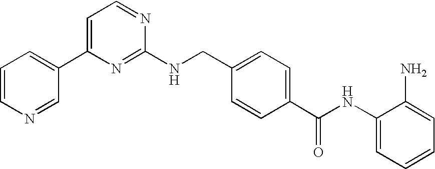 | Compound A | 0.2 | 0.3 | 1.7 | >10 | >10 | >10 | >10 | >10 |
| 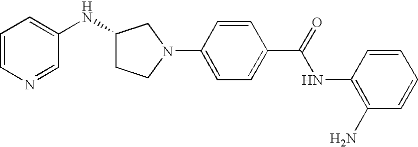 | Compound B | 0.2 | 0.5 | 0.3 | >10 | >10 | >10 | >10 | 1.63 |
| 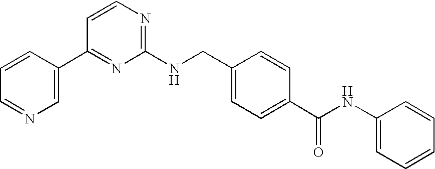 | Compound C | >10 | >10 | >10 | NT | NT | NT | NT | NT |
| 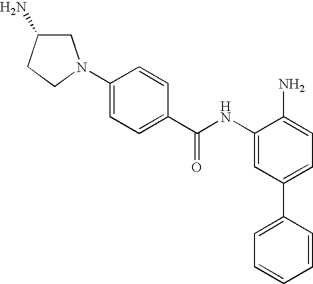 | Compound D | 0.04 | 0.09 | 9.0 | >10 | >10 | >10 | >10 | >10 |
| 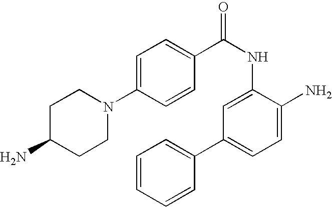 | Compound E | 0.02 | 0.08 | NT | >10 | >10 | >10 | >10 | >10 |
| 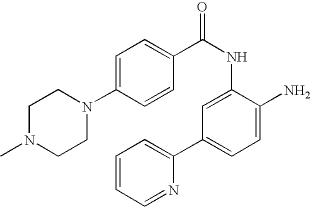 | Compound F | 0.10 | 0.10 | NT | >10 | >10 | >10 | >10 | >10 |
| 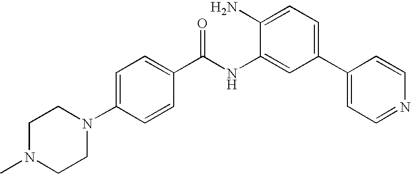 | Compound G | 0.06 | 0.10 | NT | >10 | >10 | >10 | >10 | >10 |
| 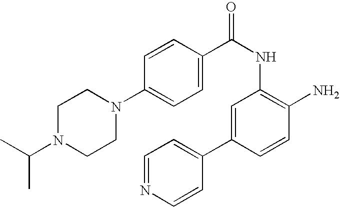 | Compound H | 0.06 | 0.09 | NT | >10 | >10 | >10 | >10 | >10 |

Of the compounds shown in Table 1, Compounds A and B are selective inhibitors of HDAC1, 2 and 3, while Compound C is an inactive compound used as a negative control. Compounds D, E, F, G, H are HDAC inhibitors that are selective for HDAC1 and HDAC 2.

Other compounds that are useful in the methods according to the invention are compounds that stabilize microtubules. Many of these compounds are taxanes, including, without limitation, Paclitaxel (taxol) and Docetaxel (taxotere). Other compounds that are useful in the methods according to the invention include, without limitation, epothilones (for example epothilone A, B and D) and epothilone analogs (for example ixabepilone).

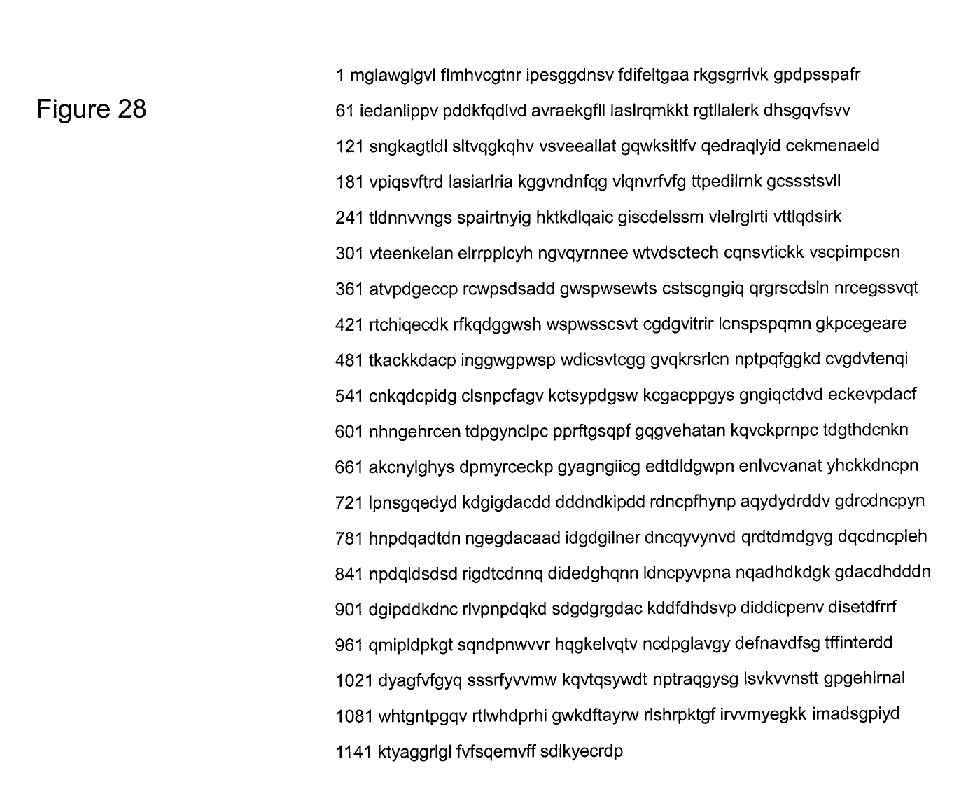

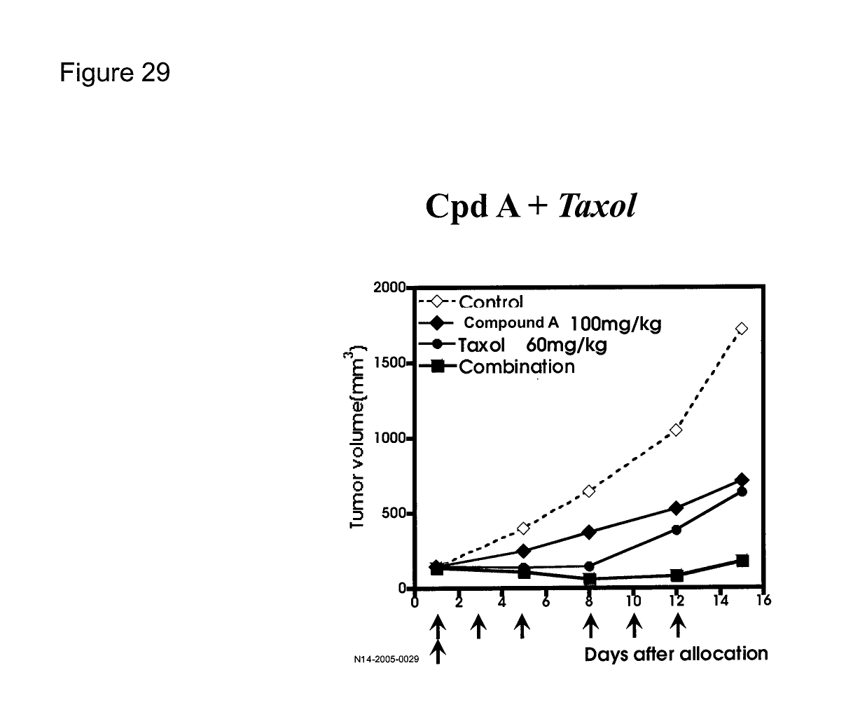

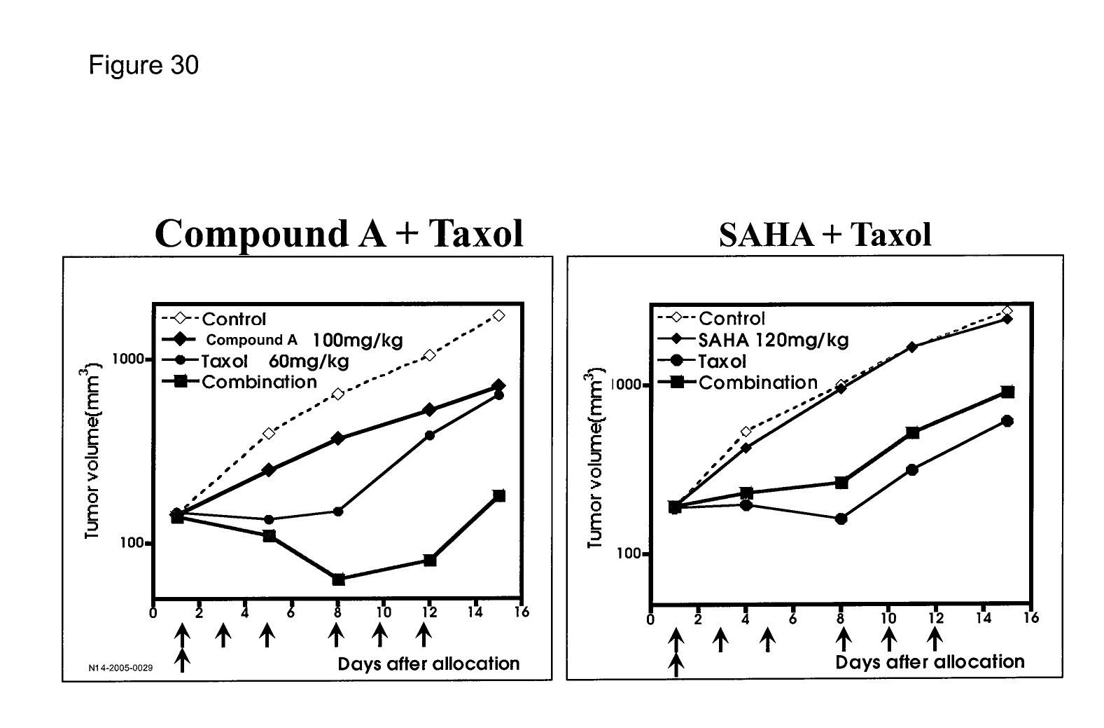

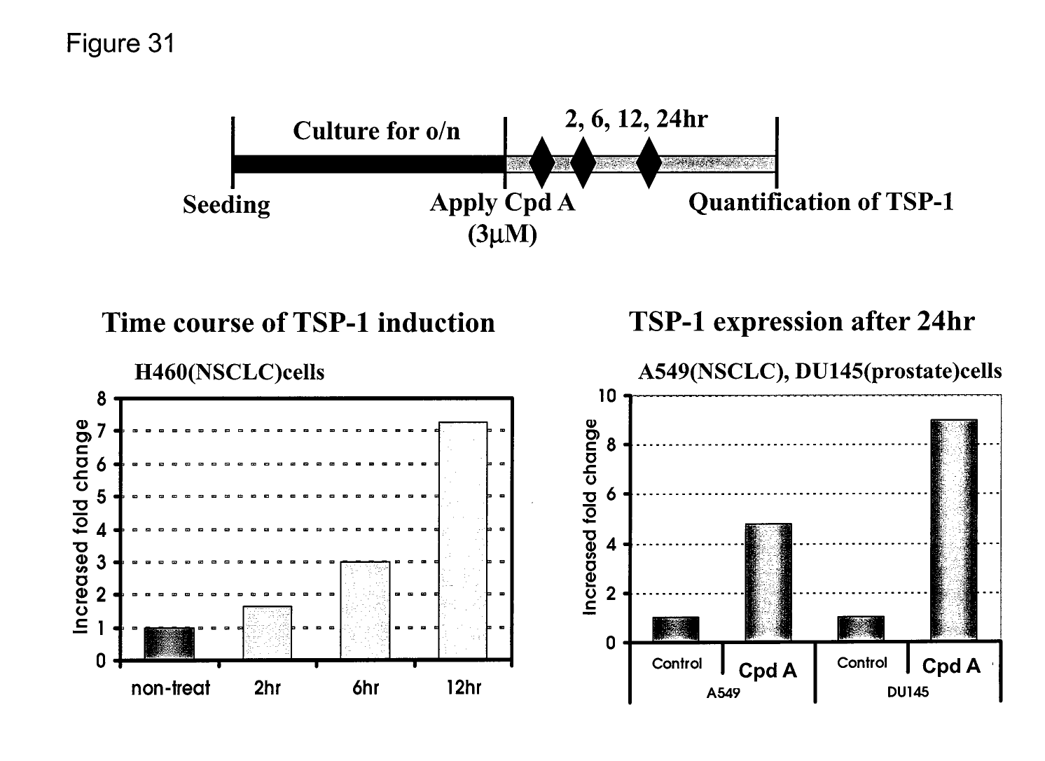

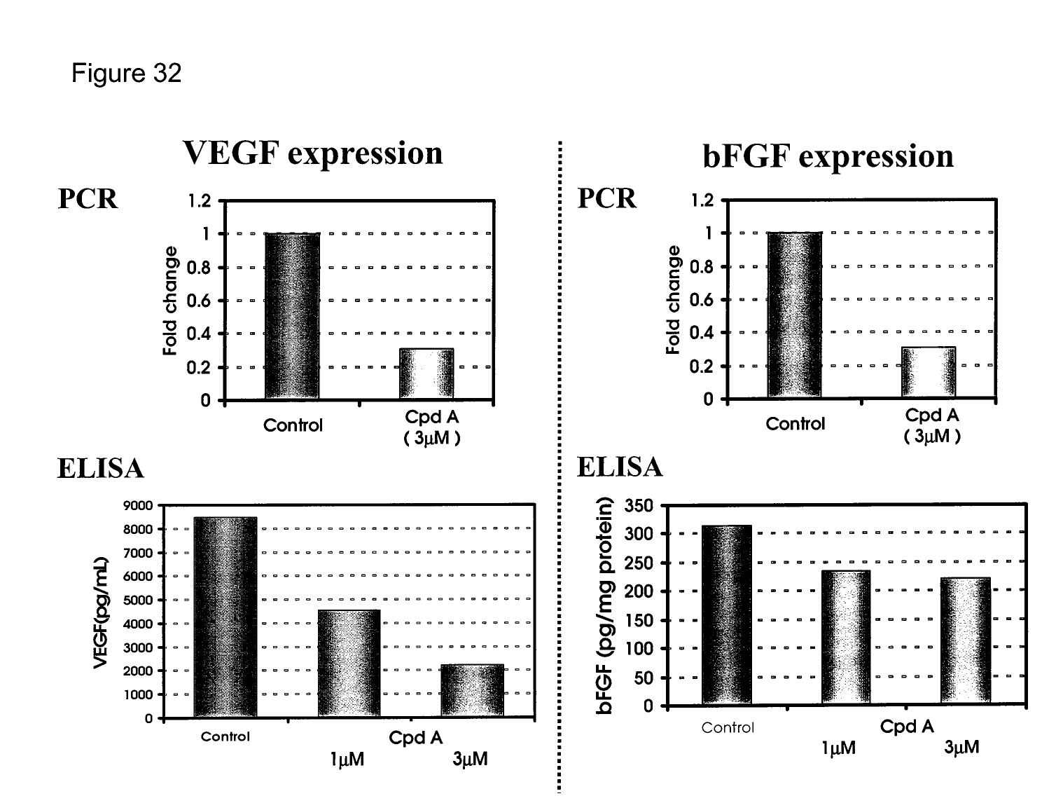

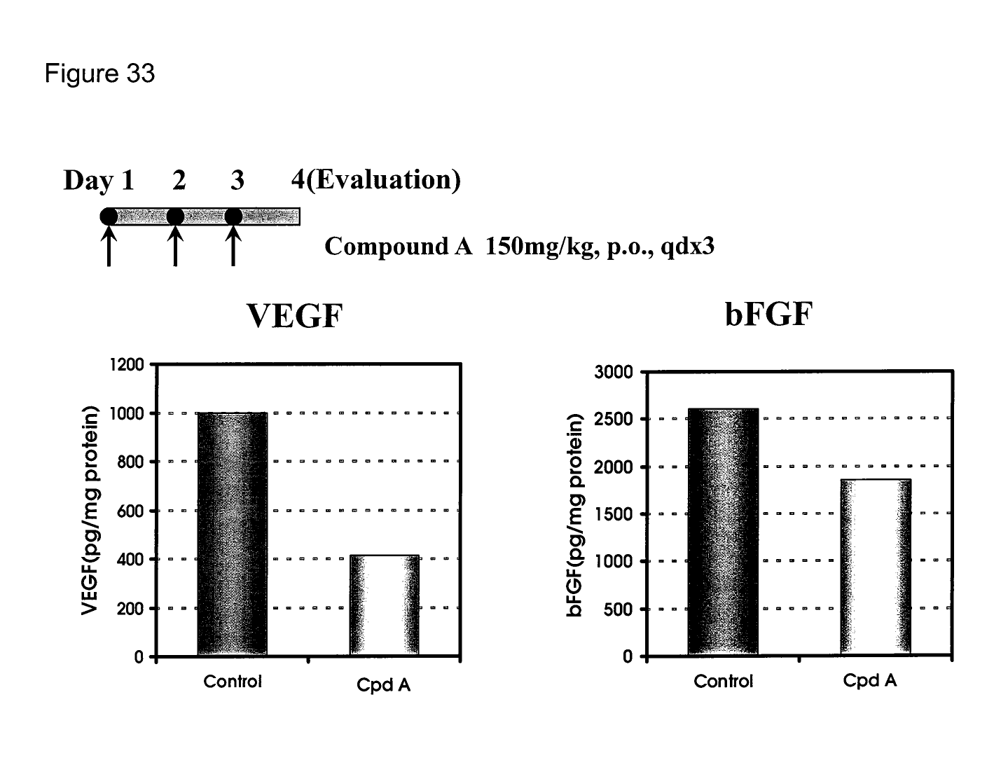

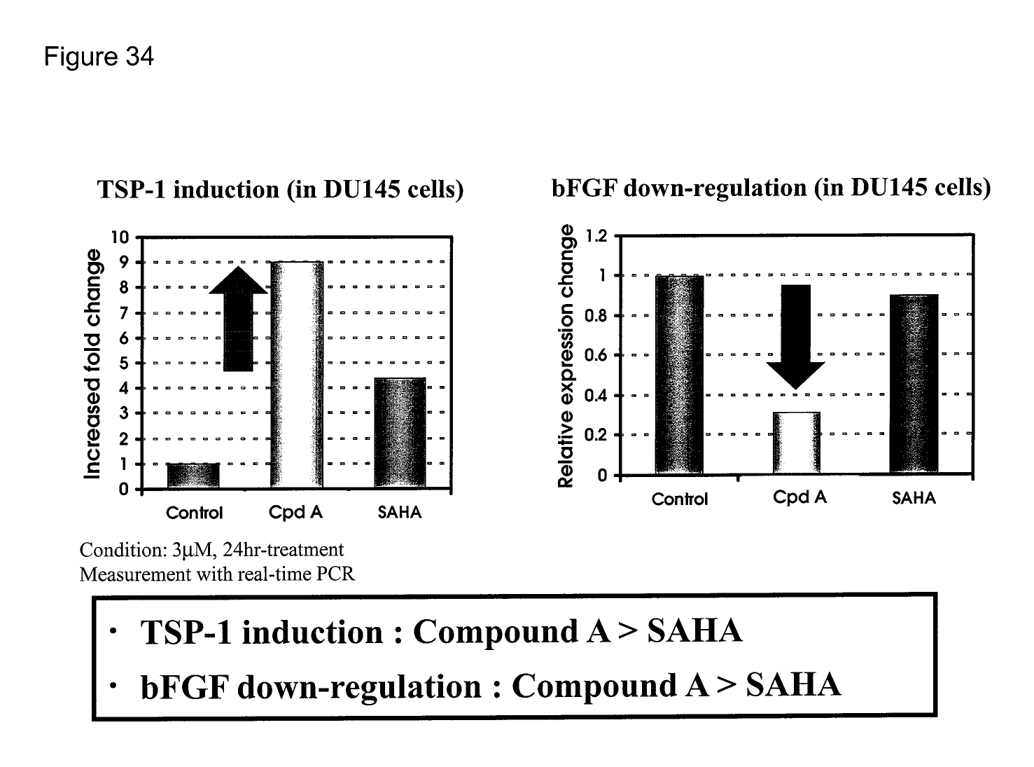

In certain embodiments, additional compounds useful in the methods according to the invention are agonists of thrompospondin-1 (TSP1) receptor, including, without limitation, recombinant TSP1 (FIG. 28) and mimetics of the active TSP1 heptapeptide, such as ABT-510, (Ac-G V _{D}I T R I R-_{Neth}, as in Dawson et al. Molecular Pharmacology (1999) 55:332-338).

In a first aspect, the invention provides a method for inhibiting abnormal cell growth and/or abnormal cell proliferation in a mammal comprising administering to a mammal in need thereof an effective amount of a selective inhibitor of histone deacetylase (HDAC)1, HDAC2 and/or HDAC3 in combination with an effective amount of a compound that stabilizes microtubules.

For purposes of this aspect of the invention a “selective inhibitor of HDAC1, HDAC2 and/or HDAC3” is a compound that inhibits the enzymatic activity of HDAC1, HDAC2 and/or HDAC3 with an IC₅₀ that is at least 5-fold, more preferably at least 10-fold lower than its IC₅₀ for any of HDAC4, HDAC5, HDAC6, HDAC7, HDAC8, HDAC9, HDAC10 and HDAC11. Preferred selective inhibitors of HDAC1, HDAC2 and/or HDAC3 include, without limitation, compounds with Formula (I), (II) and (III), such as Compound A and Compound B. A “compound that stabilizes microtubules” is a compound that inhibits disassembly of tubulin from the (−) end of a microtubule at least 2-fold, preferably at least 3-fold, more preferably at least 5-fold and more preferably still at least 10-fold greater than it inhibits assembly of tubulin at the (+) end of a microtubule. Preferred compounds that stabilize microtubules include, without limitation, taxanes, such as Paclitaxel (taxol) and Docetaxel (taxotere). Other preferred compounds include, without limitation, epothilones (for example epothilone A, B and D) and epothilone analogs (for example ixabepilone). “In combination with” means administered during the treatment of the same course of disease, which may be simultaneously or sequentially, or both simultaneously and sequentially.

In an embodiment of the first aspect, the invention provides a method for inhibiting tumor cell growth in a mammal, comprising administering to a mammal in need thereof an effective amount of a selective inhibitor of histone deacetylase (HDAC)1, HDAC2 and/or HDAC3 in combination with an effective amount of a compound that stabilizes microtubules.

In certain embodiments, a selective inhibitor of HDAC1, HDAC2 and/or HDAC3 is administered either orally or intravenously. In certain embodiments, a compound that stabilizes microtubules is administered intravenously.

In a second aspect, the invention provides a method for inhibiting abnormal cell growth and/or abnormal cell proliferation in a mammal comprising administering to a mammal in need thereof an effective amount of a selective inhibitor of histone deacetylase (HDAC)1 and/or HDAC2 in combination with an effective amount of a compound that stabilizes microtubules.

For purposes of this aspect of the invention a “selective inhibitor of HDAC1 and/or HDAC2” is a compound that inhibits the enzymatic activity of HDAC1 and/or HDAC2 with an IC₅₀ that is at least 5-fold, more preferably at least 10-fold lower than its IC₅₀ for any of HDAC3, HDAC4, HDAC5, HDAC6, HDAC7, HDAC8, HDAC9, HDAC10 and HDAC11. Preferred selective inhibitors of HDAC1 and/or HDAC2 include, without limitation, compounds with Formula (IV), (IVa) and (V), such as Compound D, Compound E, Compound F, Compound G, and Compound H. The terms “compound that stabilizes microtubules” and “in combination with” are as described for the first aspect of the invention.

In an embodiment of the second aspect, the invention provides a method for inhibiting tumor cell growth in a mammal comprising administering to a mammal in need thereof an effective amount of a selective inhibitor of histone deacetylase (HDAC)1 and/or HDAC2 in combination with an effective amount of a compound that stabilizes microtubules.

In certain embodiments, a selective inhibitor of HDAC1 and/or HDAC2 is administered either orally or intravenously. In certain embodiments, a compound that stabilizes microtubules is administered intravenously.

In a third aspect, the invention provides a method for inhibiting abnormal cell growth and/or abnormal cell proliferation in a mammal comprising up-regulating the expression of metalothionene 3 (MT3) in the cell and/or up-regulating the expression of thrombospondin-1 (TSP1) in the cell in combination with administering a compound that stabilizes microtubules.

For purposes of this aspect of the invention, “up-regulating the expression of MT3” means causing an increase of MT3 expression in the cell of at least 2-fold. “up-regulating the expression of TSP1” means causing an increase in TSP1 of at least 1.5-fold, preferably at least 1.8-fold and more preferably at least 2 or 3-fold in the cell. A “compound that stabilizes microtubules” and “in combination with” have the same meanings as in the first aspect of the invention. Such up-regulation may be measured by the level of protein, the level of mRNA encoding the protein or both. In certain preferred embodiments, up-regulating expression of MT3 and TSP1 is achieved by selectively inhibiting HDAC1, HDAC2 and/or HDAC3, preferably HDAC1 and/or HDAC2. “Selectively inhibiting HDAC1, HDAC2 and/or HDAC3” means inhibiting the enzymatic activity of HDAC1, HDAC 2 and/or HDAC3 in a cell at least 5-fold, more preferably at least 10-fold greater than the inhibition of any of HDAC4, HDAC5, HDAC6, HDAC7, HDAC8, HDAC9, HDAC10 and HDAC11 in the cell.

In an embodiment of the third aspect, the invention provides a method for inhibiting tumor cell growth in a mammal, comprising up-regulating the expression of metalothionene 3 (MT3) in the tumor cells and/or up-regulating the expression of thrombospondin-1 (TSP1) in the tumor cells and/or stromal cells in a tumor, in combination with administering a compound that stabilizes microtubules.

For purposes of this embodiment of the third aspect of the invention, “up-regulating the expression of MT3 in tumor cells” means causing an increase of MT3 expression in the tumor cell of at least 2-fold. “up-regulating the expression of TSP1 in the tumor cells and/or stromal cells in a tumor” means causing an increase in TSP1 of at least 1.5-fold, preferably at least 1.8-fold and more preferably at least 2 or 3-fold in the tumor cells, in the stromal cells within a tumor, or in both. A “compound that stabilizes microtubules” and “in combination with” have the same meanings as in the first aspect of the invention. Such up-regulation may be measured by the level of protein, the level of mRNA encoding the protein or both. In certain preferred embodiments, up-regulating expression of MT3 and TSP1 is achieved by selectively inhibiting HDAC1, HDAC2 and/or HDAC3, preferably HDAC1 and/or HDAC2. “Selectively inhibiting HDAC1, HDAC2 and/or HDAC3” means inhibiting the enzymatic activity of HDAC1, HDAC 2 and/or HDAC3 in a tumor sample at least 5-fold, more preferably at least 10-fold greater than the inhibition of any of HDAC4, HDAC5, HDAC6, HDAC7, HDAC8, HDAC9, HDAC10 and HDAC11 in the tumor sample.

In a fourth aspect, the invention provides a method for inhibiting abnormal cell growth and/or abnormal cell proliferation in a mammal comprising administering to a mammal in need thereof an effective amount of an agonist of TSP1 receptor in combination with an effective amount of a compound that stabilizes microtubules. In certain embodiments, the agonist of TSP1 receptor is selected from recombinant TSP1 and a mimetic of active TSP1 heptapeptide. In further embodiments, the mimetic of active TSP1 heptapeptide is ABT-510. In alternate embodiments of this aspect of the invention, the method further comprises administering to the mammal an effective amount of a selective inhibitor of histone deacetylase (HDAC)1, HDAC2 and/or HDAC3, as described for the first aspect of the invention. In some embodiments of this aspect of the invention, the method further comprises administering to the mammal a selective inhibitor of HDAC1 and/or HDAC2, as described for the second aspect of the invention.

In an embodiment of the fourth aspect, the invention provides a method for inhibiting tumor cell growth in a mammal comprising administering to a mammal in need thereof an effective amount of an agonist of TSP1 receptor in combination with an effective amount of a compound that stabilizes microtubules.

In a fifth aspect, the invention provides a method for inhibiting abnormal cell growth and/or abnormal cell proliferation in a mammal comprising up-regulating the expression of thrombospondin-1 (TSP1) in the cells, in combination with administering a compound that stabilizes microtubules.

For purposes of this aspect of the invention, “up-regulating the expression of TSP1 in the cells” means causing an increase in TSP1 of at least 2-fold in the cells. The terms “compound that stabilizes microtubules” and “in combination with” are as described for the first aspect of the invention.

In an embodiment of the fifth aspect, the invention provides a method for inhibiting tumor cell growth in a mammal comprising up-regulating the expression of thrombospondin-1 (TSP1) in the tumor cells and/or stromal cells in a tumor, in combination with administering a compound that stabilizes microtubules.

For purposes of this embodiment of the fifth aspect of the invention, “up-regulating the expression of TSP1 in the tumor cells and/or stromal cells in a tumor” means causing an increase in TSP1 of at least 2-fold in the tumor cells, in stromal cells within the tumor, or in both. The terms “compound that stabilizes microtubules” and “in combination with” are as described for the first aspect of the invention.

In a sixth aspect, the invention provides a method for inhibiting abnormal cell growth and/or abnormal cell proliferation in a mammal comprising administering to a mammal in need thereof an agonist of metalothionene 3 (MT3) expression in the cells and/or an agonist of thrombospondin-1 (TSP1) expression in the cellsin combination with administering a compound that stabilizes microtubules.

For purposes of this aspect of the invention, the terms “compound that stabilizes microtubules” and “in combination with” are as described above in the previous aspects of the invention.

In an embodiment of the sixth aspect, the invention provides a method for inhibiting tumor cell growth in a mammal comprising administering to a mammal in need thereof an agonist of metalothionene 3 (MT3) expression in the tumor cells and/or an agonist of thrombospondin-1 (TSP1) expression in the tumor cells and/or stromal cells, in combination with administering a compound that stabilizes microtubules.

In a seventh aspect, the invention provides a method for inhibiting angiogenesis comprising administering to a mammal a selective inhibitor of histone deacetylase (HDAC)1, HDAC2 and/or HDAC3.

For the purposes of this aspect of the invention, the term “selective inhibitor of histone deacetylase (HDAC)1, HDAC2 and/or HDAC3” is as described in the first aspect of the invention.

In an embodiment of the seventh aspect, the invention provides a method for inhibiting angiogenesis in a tumor, comprising administering to the tumor a selective inhibitor of histone deacetylase (HDAC)1, HDAC2 and/or HDAC3. In another embodiment of this aspect the tumor is treated in a mammal. In another embodiment of the seventh aspect, the tumor is in a mammal and the mammal is administered the selective inhibitor of histone deacetylase (HDAC)1, HDAC2 and/or HDAC3.

In an eighth aspect, the invention provides a method for inducing expression of an anti-angiogenesis factor in a cell, the method comprising administering to the cell a selective inhibitor of histone deacetylase (HDAC)1, HDAC2 and/or HDAC3.

In an embodiment of this aspect, the cell is in a mammal, in which case the method comprises administering to the mammal a selective inhibitor of histone deacetylase (HDAC)1, HDAC2 and/or HDAC3. In another embodiment of this aspect, the cell is a mammalian tumor cell. In another embodiment of this aspect, the cell is a mammalian tumor cell, which tumor cell is in a mammal.

For the purposes of this aspect of the invention, the term “inducing expression of an anti-angiogenesis factor” in a cell means causing an increase of expression of an anti-angiogenesis factor of at least 1.5-fold, preferably at least 1.8-fold and more preferably at least 2 or 3-fold in the cell. In a preferred embodiment of this aspect of the invention, the anti-angiogenesis factor is TSP1.

For the purposes of this aspect of the invention, the term “selective inhibitor of histone deacetylase (HDAC)1, HDAC2 and/or HDAC3” is as described in the first aspect of the invention.

In a ninth aspect, the invention provides a method for inhibiting expression of an angiogenesis factor in a cell, the method comprising administering to the cell a selective inhibitor of histone deacetylase (HDAC)1, HDAC2 and/or HDAC3.

In an embodiment of this aspect, the cell is in a mammal, in which case the method comprises administering to the mammal a selective inhibitor of histone deacetylase (HDAC)1, HDAC2 and/or HDAC3. In another embodiment of this aspect, the cell is a tumor cell. In another embodiment of this aspect, the cell is a tumor cell, which tumor cell is in a mammal.

For the purposes of this aspect of the invention, the term “inhibiting expression of an angiogenesis factor” in a cell means causing an decrease of expression of an angiogenesis factor of at least 1.5-fold, preferably at least 1.8-fold and more preferably at least 2 or 3-fold in the cell. In an embodiment of this aspect of the invention, the angiogenesis factor is bFGF. For the purposes of this aspect of the invention, the term “selective inhibitor of histone deacetylase (HDAC)1, HDAC2 and/or HDAC3” is as described in the first aspect of the invention.

In a tenth aspect, the invention provides a method for treating disease manifested by abnormal cell growth and/or abnormal cell proliferation in a patient comprising administering to a patient in need thereof a selective inhibitor of histone deacetylase (HDAC)1, HDAC2 and/or HDAC3 in combination with a compound that stabilizes microtubules.

For the purposes of this aspect of the invention, the terms “selective inhibitor of histone deacetylase (HDAC)1, HDAC2 and/or HDAC3” and “compound that stabilizes microtubules” are as described in the first aspect of the invention.

In an embodiment of the tenth aspect, the invention provides a method for treating cancer in a patient comprising administering to a patient in need thereof a selective inhibitor of histone deacetylase (HDAC)1, HDAC2 and/or HDAC3 in combination with a compound that stabilizes microtubules.

In an eleventh aspect, the invention provides a method for treating disease manifested by abnormal cell growth and/or abnormal cell proliferation in a patient comprising administering to a patient in need thereof a selective inhibitor of histone deacetylase (HDAC)1 and/or HDAC2 in combination with a compound that stabilizes microtubules.

For the purposes of this aspect of the invention, the terms “selective inhibitor of histone deacetylase (HDAC)1 and/or HDAC2” and “compound that stabilizes microtubules” are as described in the second aspect of the invention.

In an embodiment of the eleventh aspect, the invention provides a method for treating cancer in a patient comprising administering to a patient in need thereof a selective inhibitor of histone deacetylase (HDAC)1 and/or HDAC2 in combination with a compound that stabilizes microtubules.

In a twelfth aspect, the invention provides the use of a selective inhibitor of histone deacetylase (HDAC)1, HDAC2 and/or HDAC3 in combination with a compound that stabilizes microtubules for the manufacture of a medicament to inhibit abnormal cell growth and/or abnormal cell proliferation or to treat cancer in a patient.

For the purposes of this aspect of the invention, the terms “selective inhibitor of histone deacetylase (HDAC)1, HDAC2 and/or HDAC3” and “compound that stabilizes microtubules” are as described in the first aspect of the invention.

In am embodiment of the twelfth aspect, the invention provides the use of a selective inhibitor of histone deacetylase (HDAC)1, HDAC2 and/or HDAC3 in combination with a compound that stabilizes microtubules for the manufacture of a medicament to inhibit tumor cell growth or to treat cancer in a patient.

Methods described and claimed herein are contemplated and now expected to be efficacious for the treatment of mammalian diseases manifested by abnormal cell growth and/or otherwise abnormal cell proliferation including, but not limited to, for example, cancer, such as, melanoma, myelodysplastic syndromes (MDS), leukemia, myelogenous leukemia, lymphocytic leukemia, myeloma, colon cancer, ovarian cancer, prostate cancer, small cell lung cancer, non-small cell lung cancer, glioblastoma multiforme (brain cancer), and breast cancer.

The following examples are intended to further illustrate certain preferred embodiments of the invention and are not intended to limit the scope of the invention in any way.

### Example 1

### Production of Recombinant HDAC Isotypes

cDNAs of human HDAC1-8 and 11 were generated by RT-PCR reactions using primers complementary to the 5′ and 3′ coding sequence of human HDAC gene sequences in GenBank. cDNAs corresponding to the full length human HDAC1, 2, 3 and 11 were cloned into pBlueBac4.5 vector (Invitrogen). The constructs were used to generate recombinant baculoviruses using the Bac-N-Blue™ DNA according to the manufacturer's instructions (Invitrogen). The recombinant HDAC1, 2, 3, 11 proteins produced harbor a FLAG tag at their C-termini. cDNAs encoding truncated versions of HDAC4, 5 and 7 encompassing their deacetylase domains were cloned into pDEST10 as an N-terminal hexahistidine fusion protein and recombinant baculoviruses were generated using the Bac-to-Bac™ Baculovirus expression system (Invitrogen). HDAC6 and 8 were cloned as full length N-terminally His-tagged protein. All HDAC proteins were expressed in insect Sf-9 cells (Spodoptera frugiperdai) upon infection with recombinant baculovirus. HDAC1 enzyme was purified from the Q-sepharose FF column (Amersham Pharmacia Biotech, Baie d'Urfe QC, Canada) followed by an anti-FLAG immunoaffinity column (Sigma). HDAC2, 3 and 11 were purified using Flag-antibody immunoaffinity purification. HDAC4, 5, 6, 7 and 8 were purified using either Ni-NTA resin (QIAGEN Mississauga ON, Canada) or His-Select resin (Sigma) with step washes and elution with different concentrations of imidazole in Buffer containing 25 mM Tris (or NaPO₄) pH 8.0, 10% glycerol and 150 mM or 500 mM NaCl.

### Example 2

### Fluorescence-Based HDAC Enzyme Assay Using Recombinant HDAC Enzymes

Recombinant HDAC enzymes were incubated with diluted compounds in assay buffer (25 mM Hepes, pH 8.0, 137 mM NaCl, 1 mM MgCl₂ and 2.7 mM KCl) for 10 minutes at ambient temperatures in black 96-well plate. Boc-Lys(Ac)-AMC (for HDAC1, 2, 3, 6, and 8 enzymes), which was purchased from Bachem Biosciences Inc., (King of Prussia, Philadelphia) were added into enzyme-compound mixture and incubated at 37° C. For HDAC4, 5, 7 assays, Boc-Lys(TFA)-AMC, which was synthesized in house, was used as substrate and 0.1% BSA was added to the buffer. The final concentration of substrates was 2 times over Ki of each isotype enzyme (between 70 uM to 200 uM). Reaction time was predetermined to ensure that reaction was linear for the incubation time. Reaction was stopped by adding a freshly prepared trypsin (1 mg/ml final concentration) with 1 μM TSA (Biomol) in assay buffer. After 30 minutes, fluorescence was measured using a fluorometer (SPECTRAMAX GeminiXS, Molecular Devices, Sunnylvale, Calif.). The 50% inhibitory concentrations (IC₅₀) for inhibitors were determined by analyzing dose-response inhibition curves.

### Example 3

### Cell-Based ELISA for H3 and Tubulin Acetylation

Bladder carcinoma T24 cells were seeded in black plates with clear bottoms (Costar #3603) at 1×10⁴ cells per well in a volume of 100 μl per well, and were allowed to settle for one day at 37° C. in a CO₂ incubator. The cells were treated for 16 h with various concentrations of HDAC inhibitors. 3 h before the end of the treatment, Alamar Blue (BioSource) was added to monitor cell viability according to the manufacturer's instructions. At the end of the treatment time, Alamar Blue OD (at 570 nm and 600 nm) was recorded, then the cells were carefully washed in PBS, fixed in pre-chilled methanol for 10 min at −20° C., washed again twice in PBS, and blocked in PBS containing 0.1% Triton X-100 and 1% BSA for a minimum of 30 min. For H3 acetylation, rabbit-anti-acetyl-H3 (Upstate #06-599) was used as primary antibody at a dilution of 1:1000 for 45 min; the secondary antibody was HRP-coupled goat-anti-rabbit (Sigma #A-0545), used at 1:8000 for 45 min. For tubulin acetylation, the primary antibody was mouse anti-acetyl-tubulin (Sigma #T-6793, 1:2000, 45 min) while the secondary was HRP-coupled goat-anti-mouse antibody (Sigma #A-2304, 1:8000, 45 min). All antibodies were diluted in blocking buffer, and the cells were washed in blocking buffer following each antibody incubation. After the final wash, the bound HRP-coupled antibodies were revealed with Amplex-Red (Invitrogen) according to the manufacturer's instructions. The fluorescent signal for acetylation was normalized by dividing with the viability data obtained from Alamar Blue. EC₅₀ was defined as the concentration of compound which gave a signal half-way between the basal (untreated) level and the maximum level generated by high doses of the HDAC pan-inhibitor NVP-LAQ-824.

### Example 4

### Reduction of Transcription of VEGF and Angiogenesis Factor bFGF In Vitro by Compound A and SAHA in Human Prostate Cancer Du145 Cells

Human prostate cancer Du145 cells were exposed to Cpd A or SAHA (3 microM) for 24 hours. Total RNA was collected by Isogen (Nippongene, Tokyo, Japan) and reverse-transcribed to cDNA by ExScript® RT reagent Kit (TAKARA, Kyoto, Japan). The expression level of bFGF mRNA was detected by ABI7700 analyzer using probe/primer pre-mixture reagent (ABI, Cat# Hs00266645_m1, CA, USA) and TaqMan® Universal PCR Master Mix (ABI, Cat# 430-4437) as described in the ABI's protocol. The expression level of VEGF mRNA was detected in a similar manner using the appropriate probe/primer reagents.

### Example 4a

### Induction of Transcription of Angiogenesis Factor TSP-1 In Vitro by Compound a or SAHA in Human Prostate Cancer Du145 Cells, Human H460 Non-Small Cell Lung Cancer Cells and Human A549 Non-Small Cell Lung Cancer Cells

Cells were exposed by Cpd A or SAHA (3 microM) for 24 hours. Total RNA was collected by Isogen (Nippongene, Tokyo, Japan) and reverse-transcribed to cDNA by ExScript® RT reagent Kit (TAKARA, Kyoto, Japan). The expression level of TSP-1 mRNA was detected by ABI7700 analyzer using a probe/primer pre-mixture reagent (ABI, Cat#, CA, USA) and TaqMan® Universal PCR Master Mix (ABI, Cat# 430-4437) as described in the ABI's protocol.

### Example 4b

### Induction of Transcription of Anti-Angiogenesis Genes in Colon Adenocarcinoma HCT15 Cells by Compound A Using Microarray Analysis

Human colon cancer HCT15 cells were treated for 24 h with 1 μM Compound A. Microarray gene analysis: Total RNA was extracted using RNeasy Mini kit (Qiagen). RNA labeling, microarray hybridization, scanning and analysis were performed by Genotypics (India). RNAs were labeled with either Cy3 or Cy5 using Agilent's optimized labeling kits and hybridized to Human whole genome 44K Oligo Microarray. Array chips were ordered from Agilent (Palo Alto, Calif.). Slides were scanned using a DNA microarry scanner from Agilent and the raw data was extracted using Agilent's image analysis tool (feature extraction software). Normalization and Statistical analysis were performed using GeneSpring software. Biological analysis was performed using Biointerpreter software.

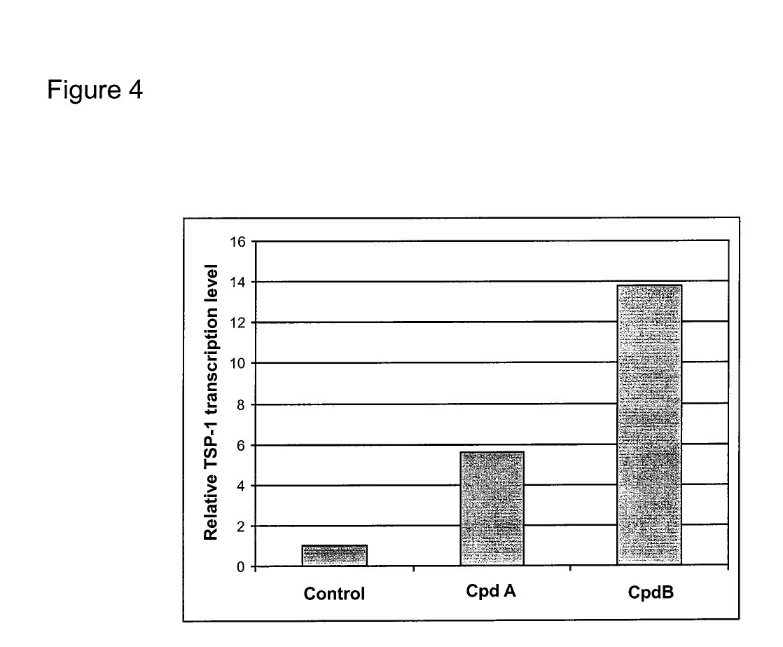

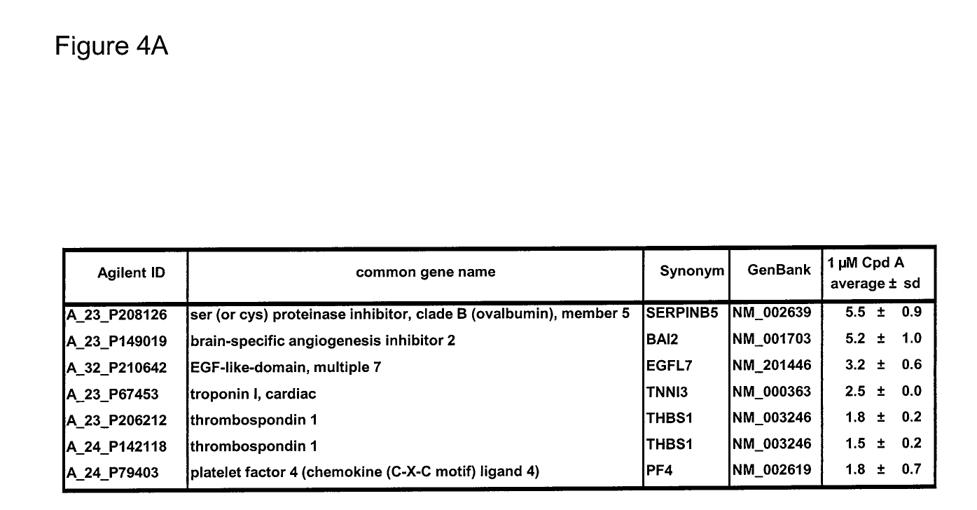

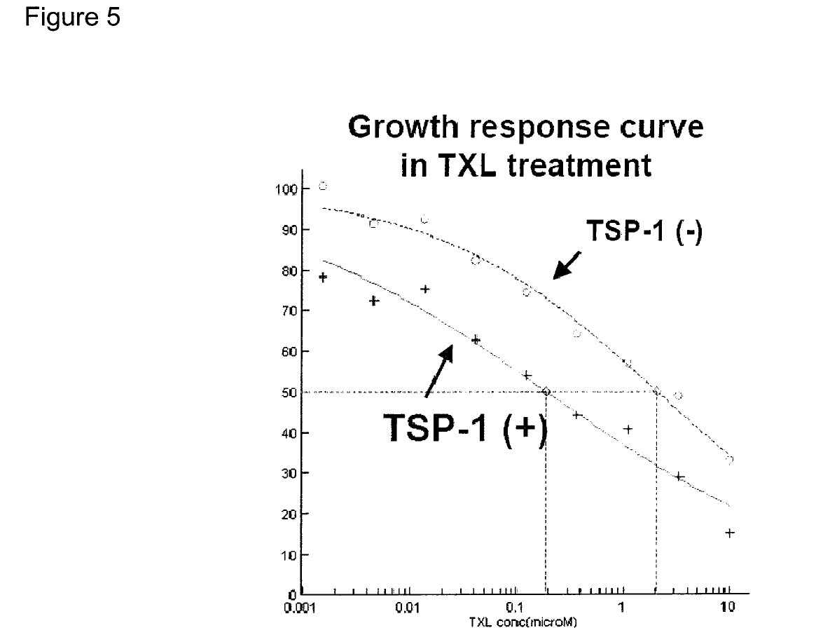

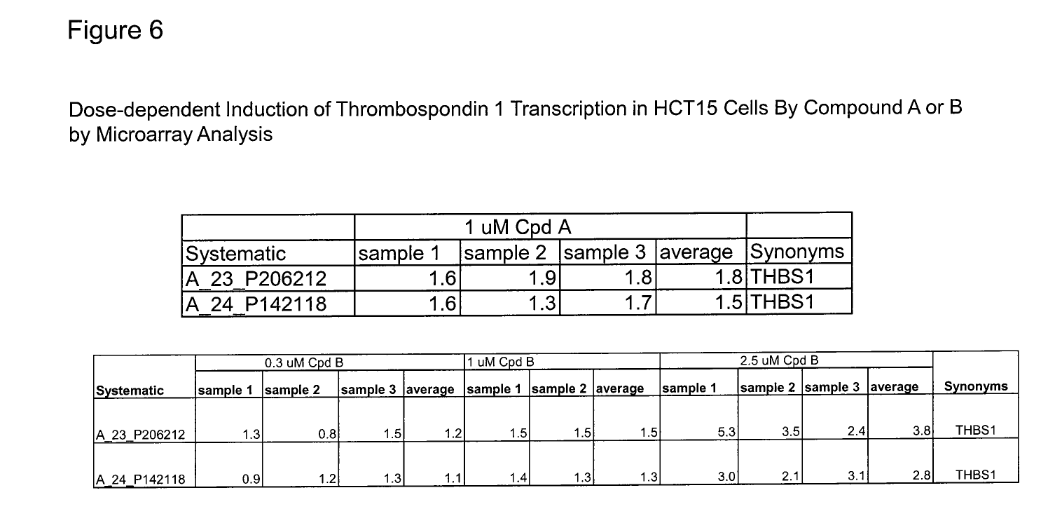

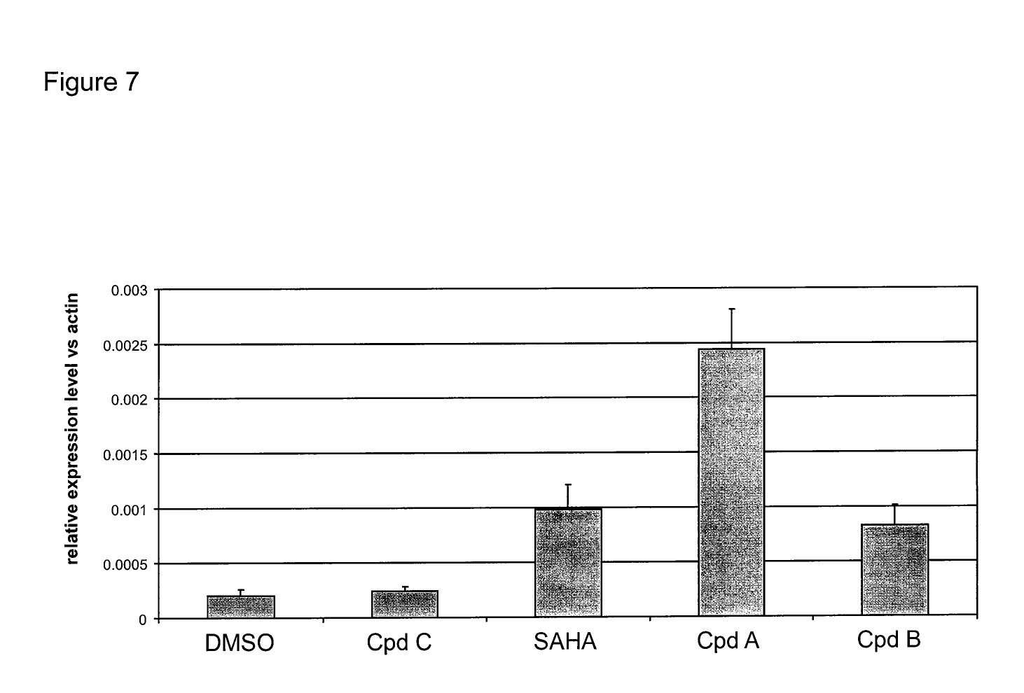

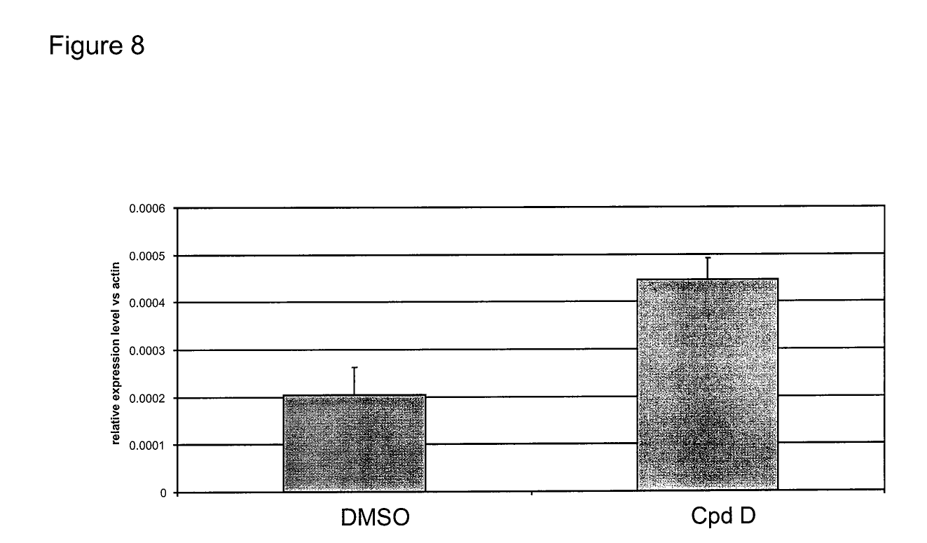

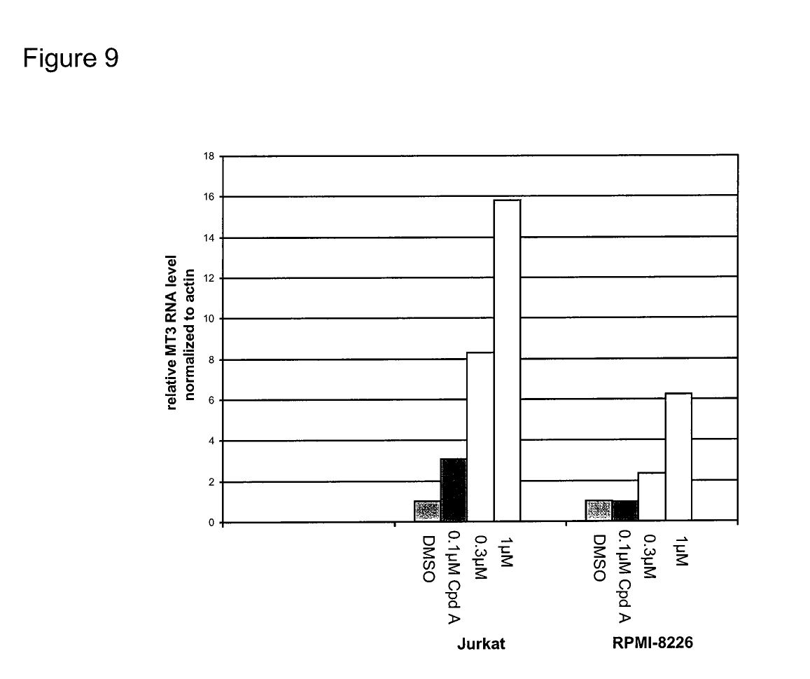

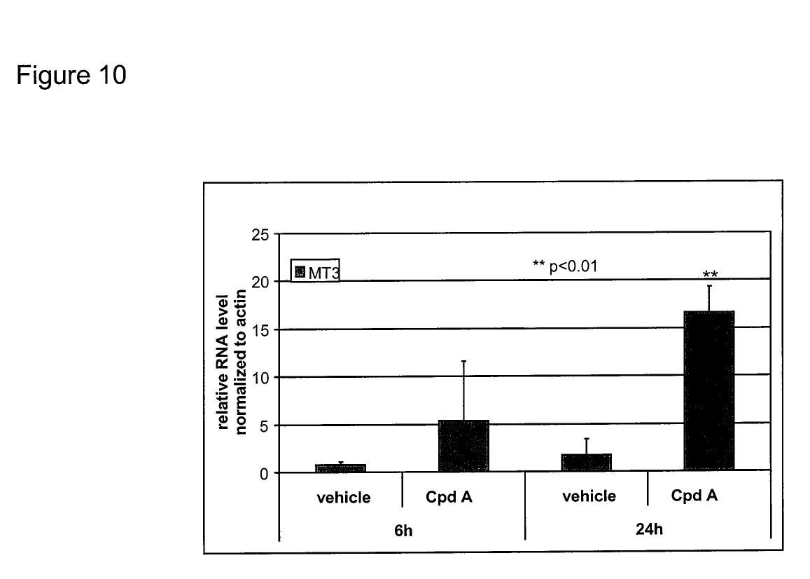

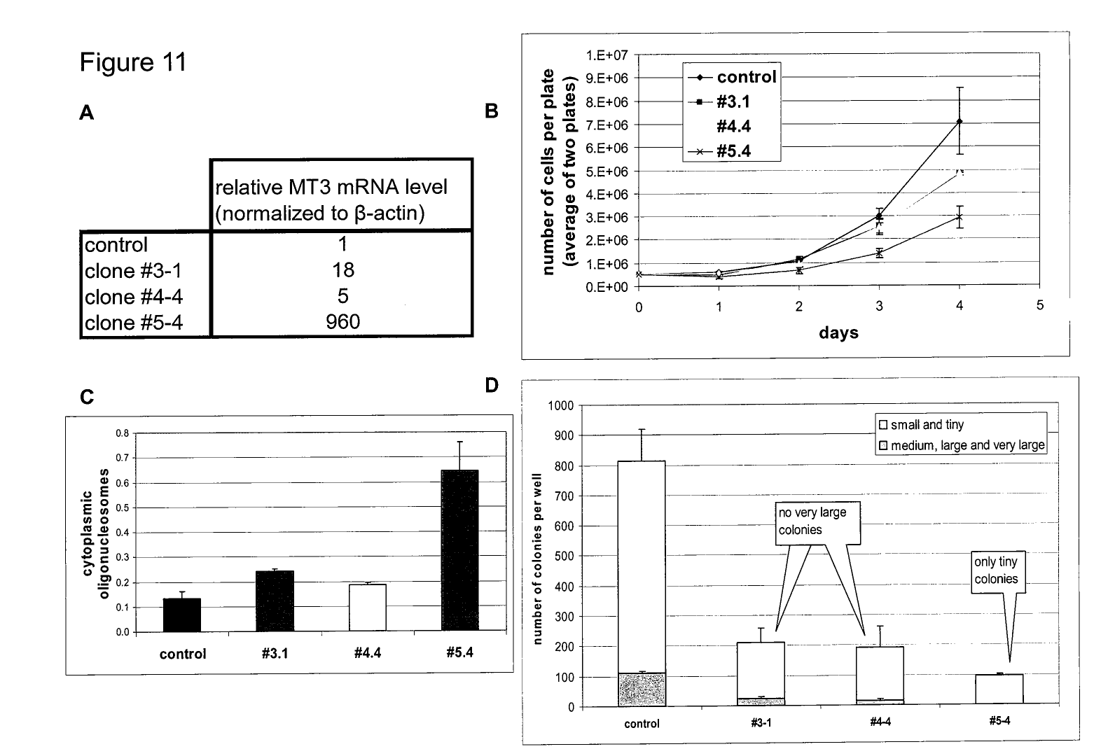

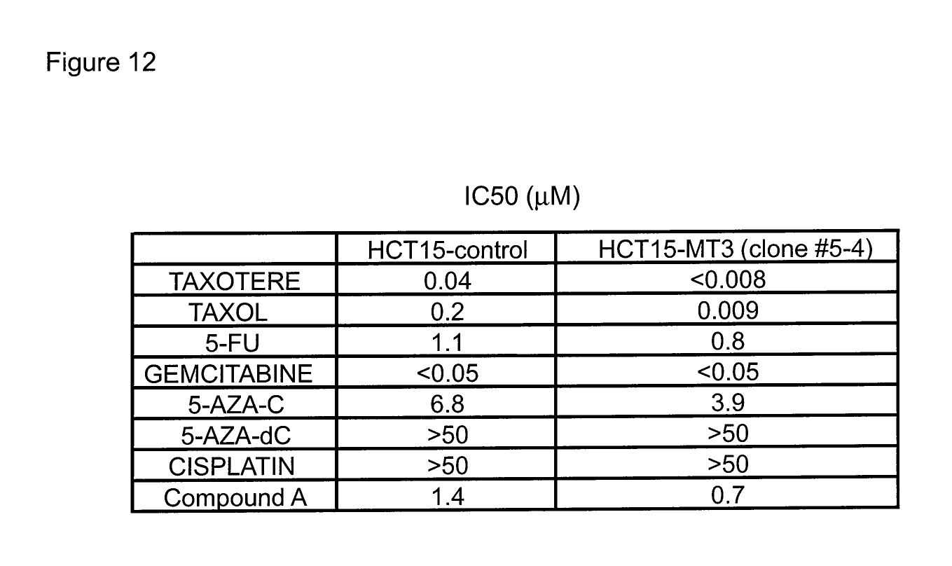

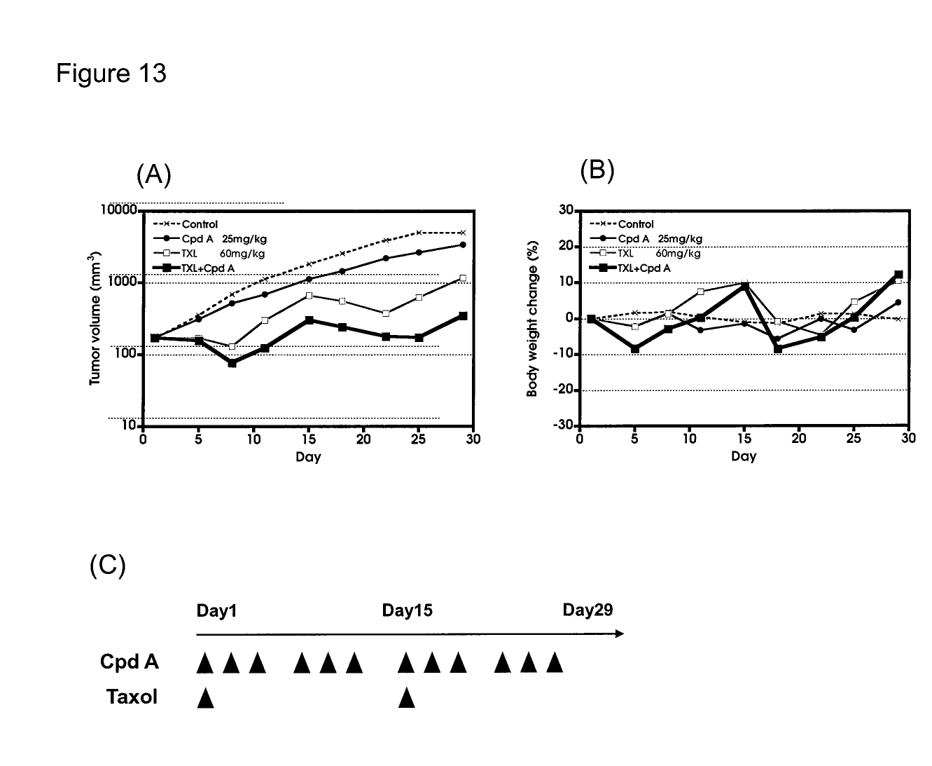

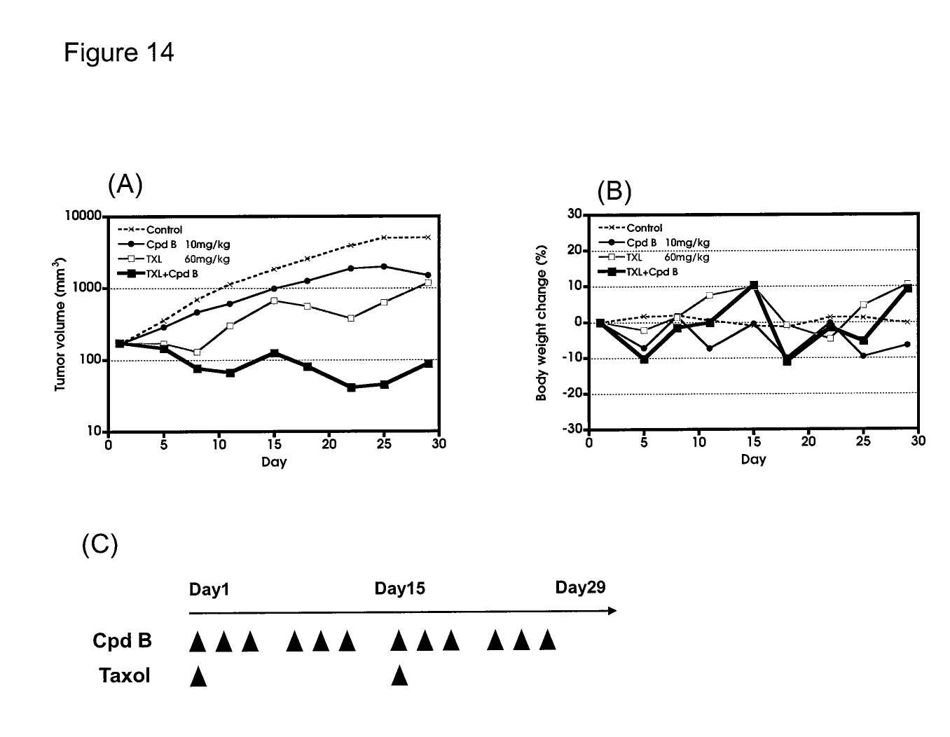

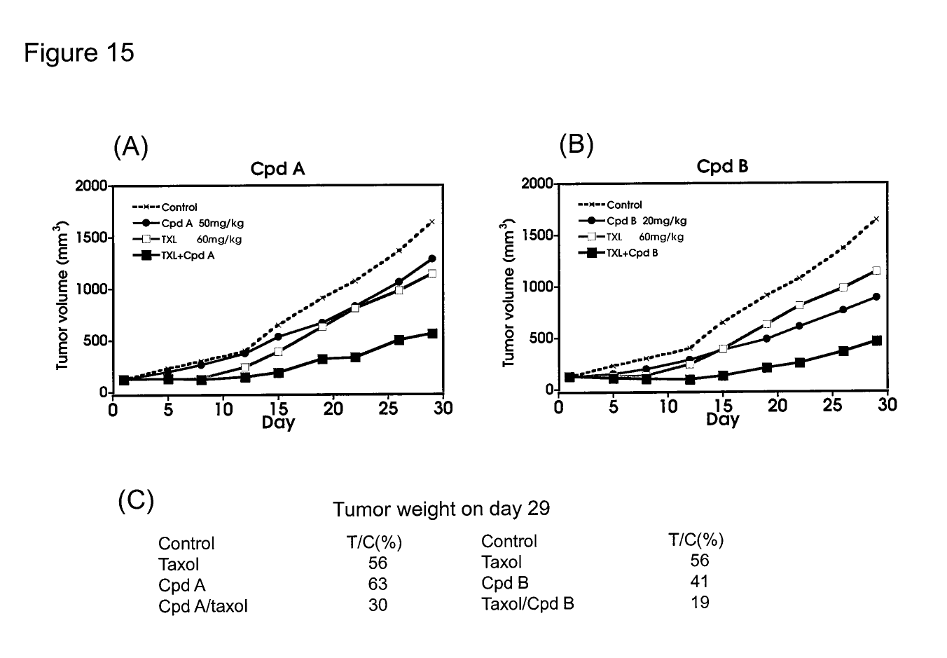

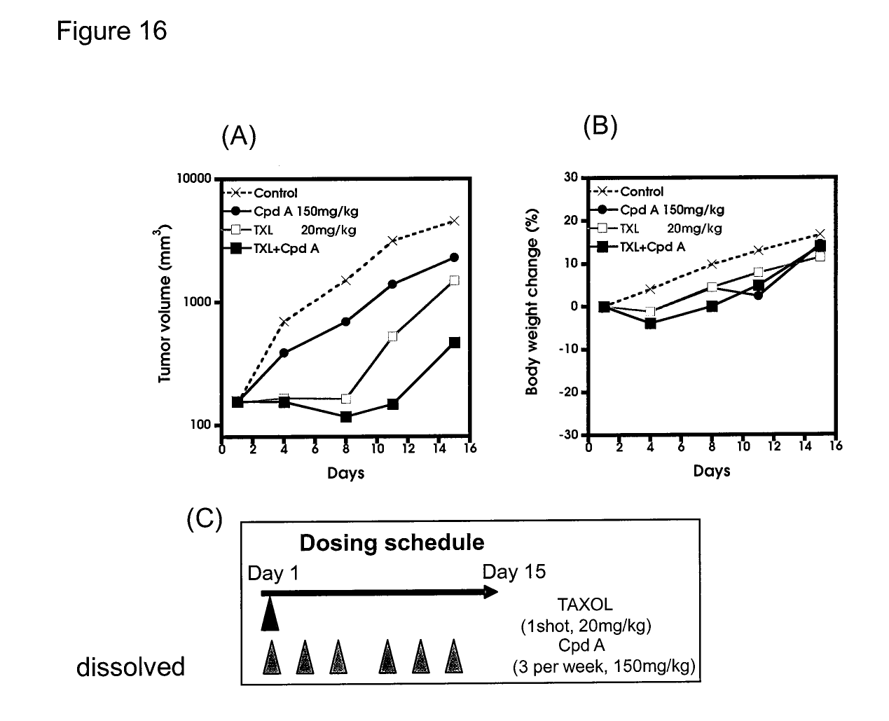

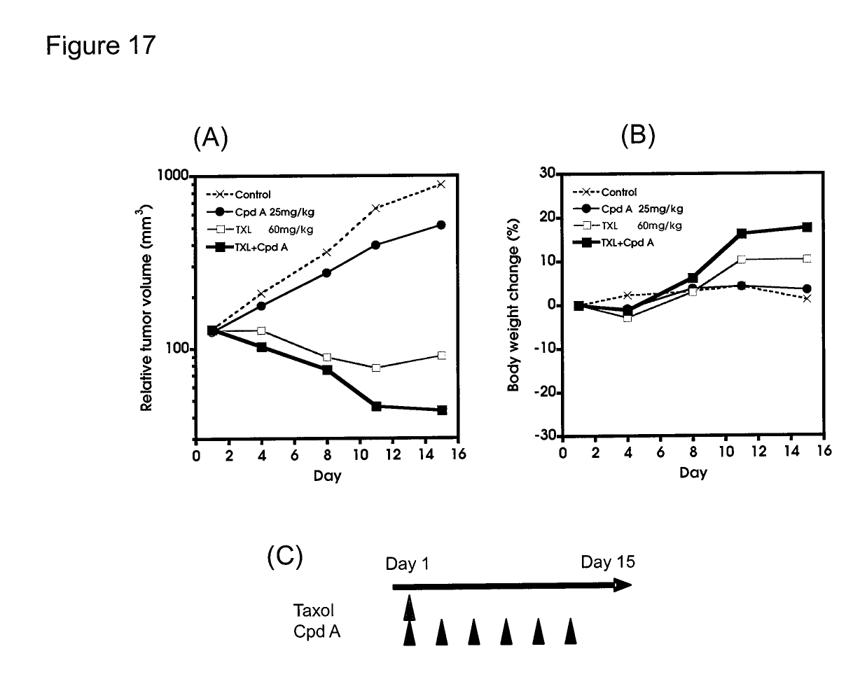

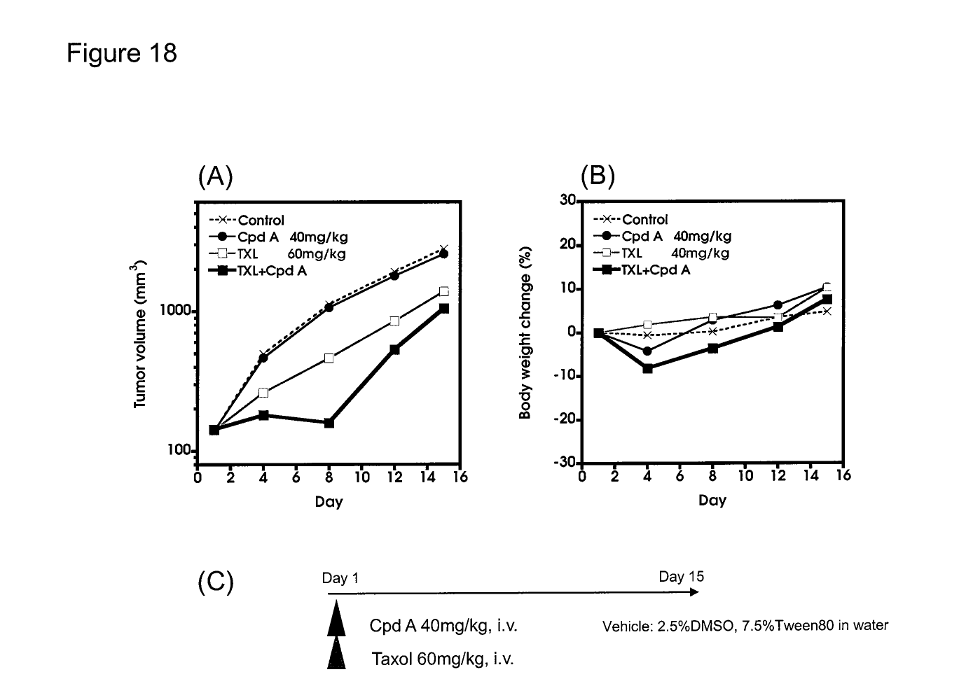

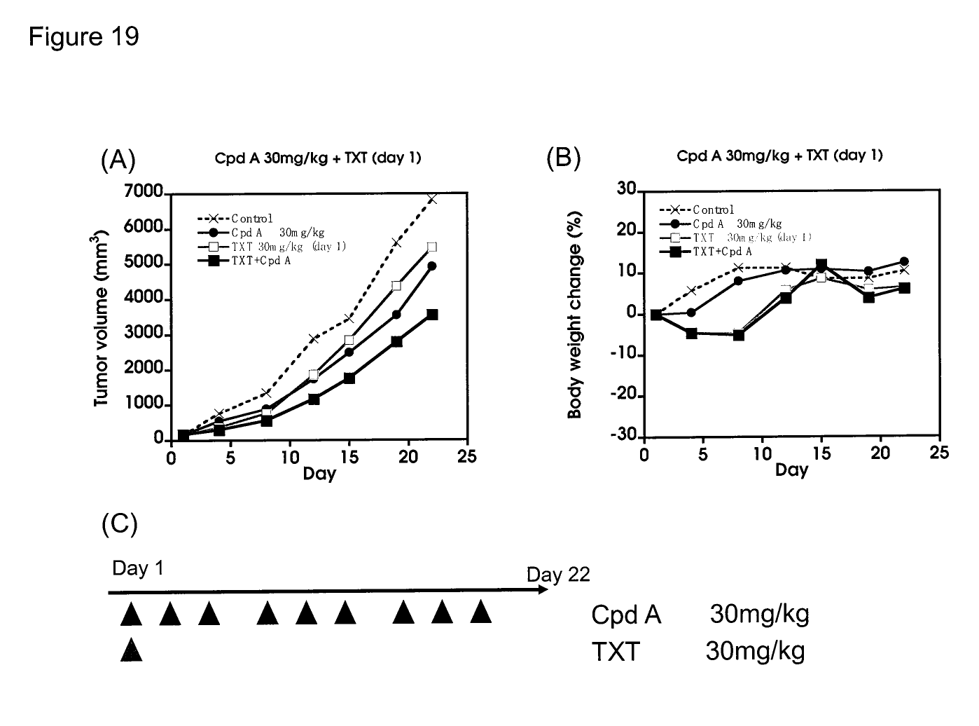

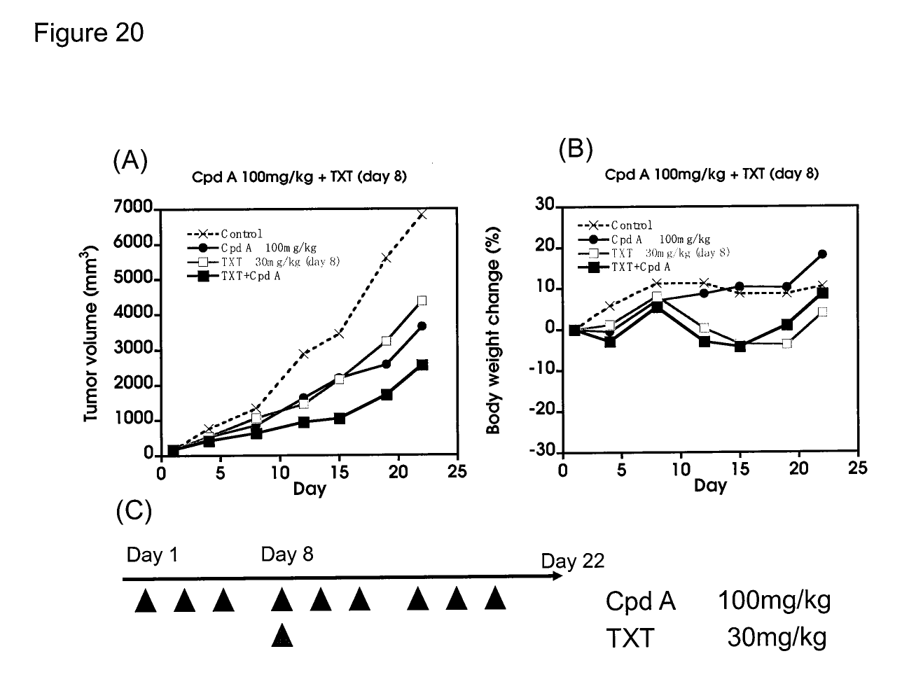

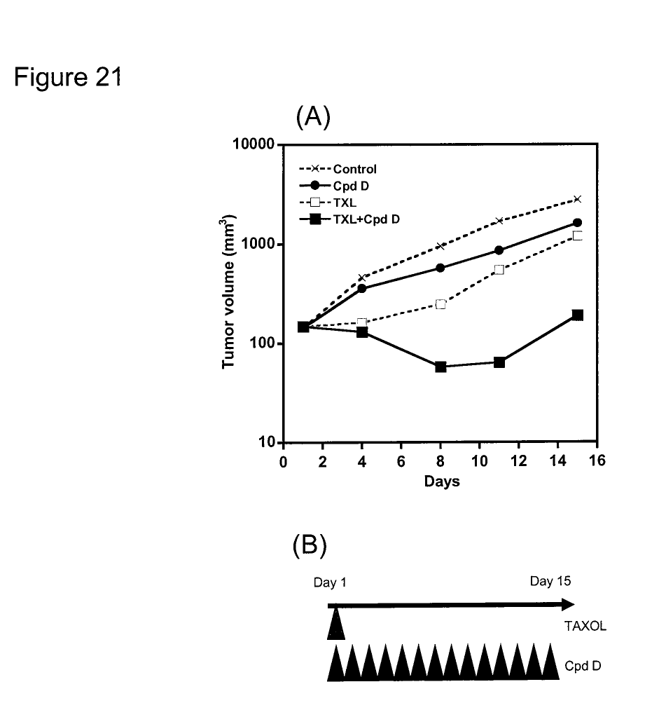

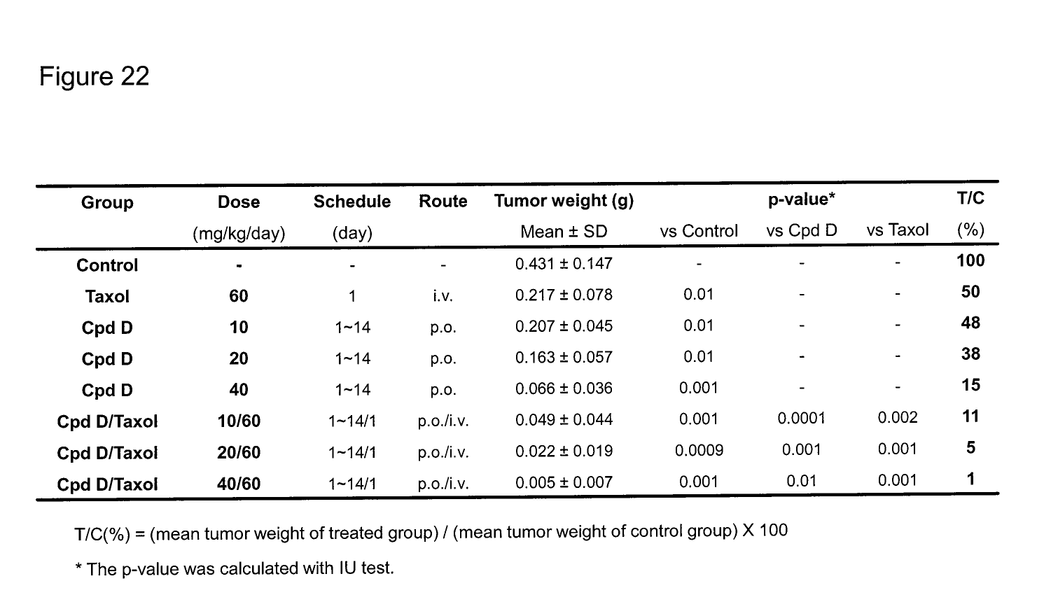

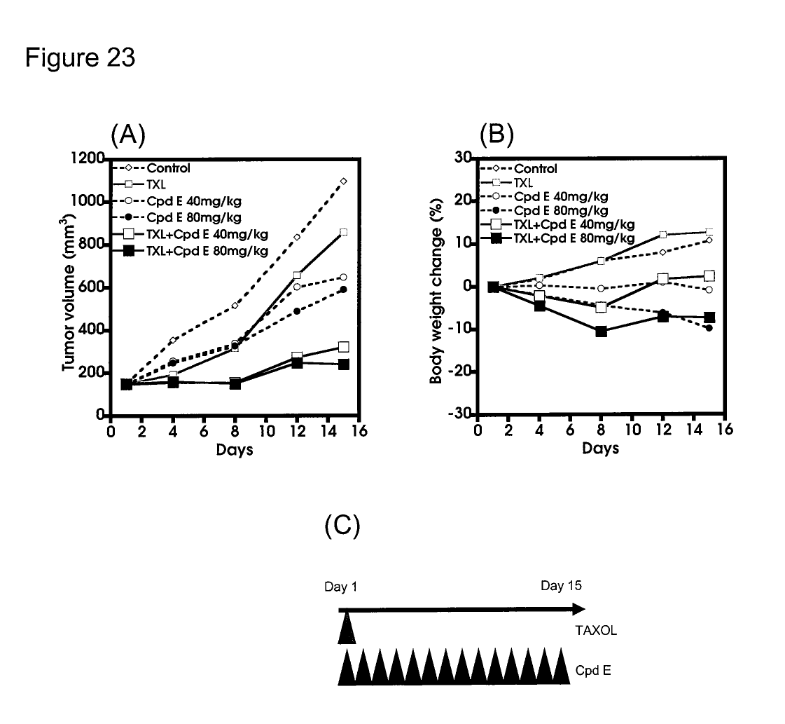

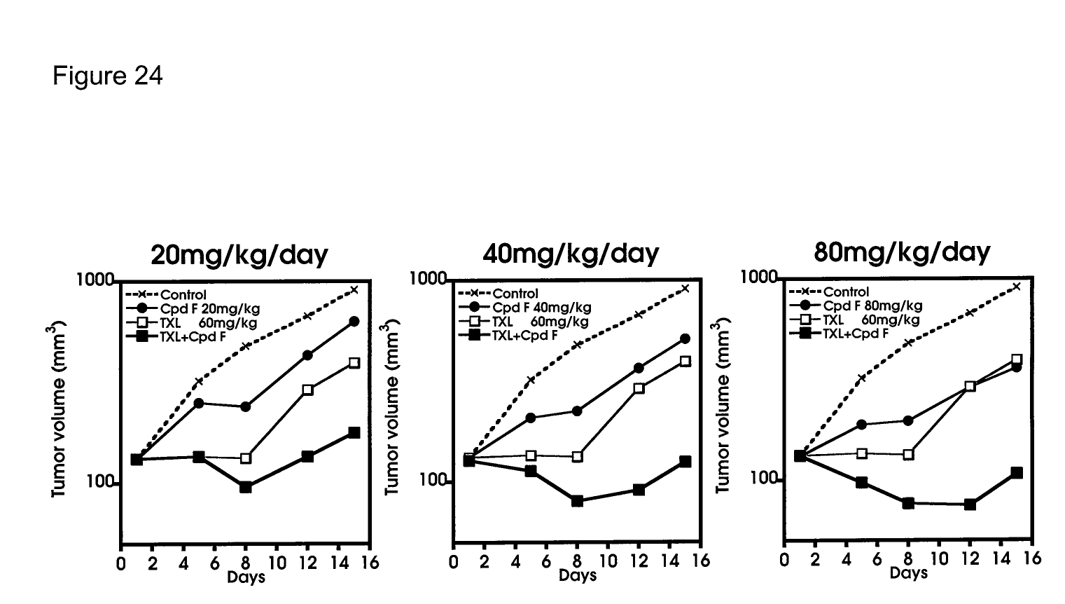

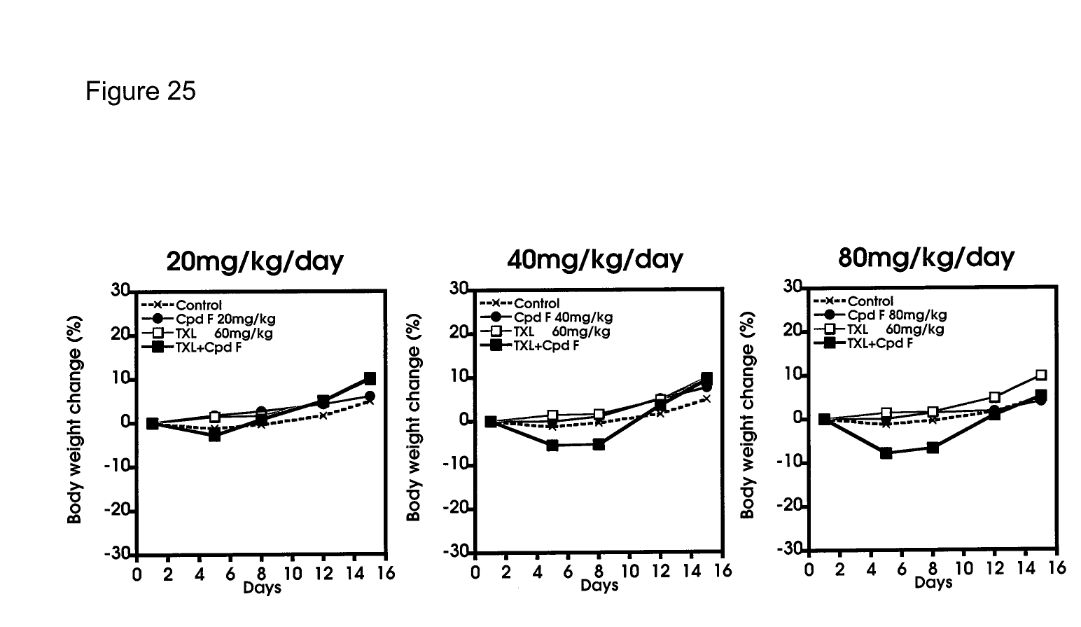

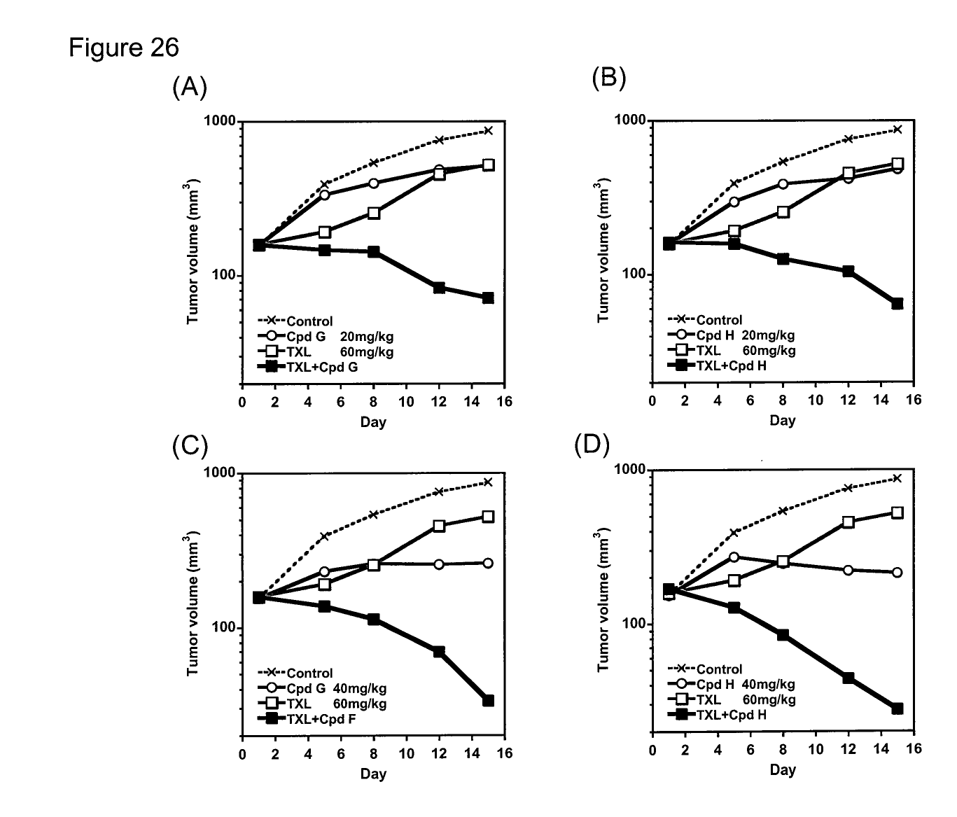

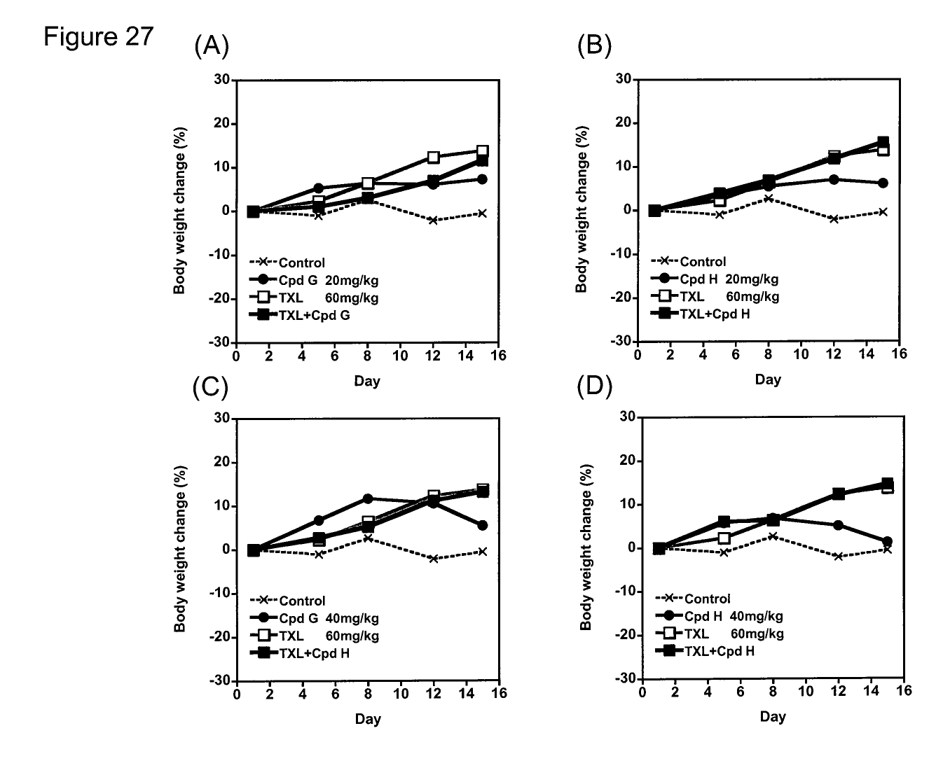

Microarray analysis revealed that Compound A affects several genes of the angiogenesis pathway. FIG. 4a indicates a list of selected genes with anti-angiogenesis function. The numbers indicate the fold induction in treated samples compared to non-treated samples (average of three biological replicates ±standard deviation).

### Example 5

### Anti-Angiogenesis Effect of Compound a In Vitro in a Human Multicellular Angiogenesis Model

Anti-angiogenesis effect of Compound A was analyzed in vitro using a human multicellular angiogenesis model, AngioKit, from TCS Cellworks, Buckingham, U.K. The AngioKits which contains co-cultured human endothelial cells were prepared by TCS Cellworks(Buckingham, UK). Briefly, 24 well plates were seeded with cells on day 0 and medium was changed on days 3, 4, 7, 10 and 12. Compound A at the appropriate dilutions (30, 100 and 300 nM) were included in the medium changes on days 4, 7, 10 and 12. ‘Untreated’ control wells were included in each plate as were wells containing DMSO (0.05%), DMSO and 20 μM suramin (negative control), and DMSO and 2 ng/ml VEGF (positive control). All AngioKits were then fixed and stained on day 14, using the CD31 Staining Kits according to the standard AngioKit procedure. Comparison of tubule development was conducted using the “AngioSys” image analysis system developed specifically for the analysis of images produced using the AngioKit. Four images taken from predetermined positions within each well were recorded. Each concentration of test compound therefore yielded 4 images for analysis in duplicate. Images were always taken from as close to the centre of each quadrant as possible. Four tubule parameters were measured: total tubule length, total tubule area, number of branch points and number of tubules formed. All statistical analyses were carried out using the Stat 100 programme from BIOSOFT Ltd. using ANOVA and Duncan's Multiple Comparison Test to measure differences between the test compounds with the untreated control values. Alpha was always 0.05 unless otherwise stipulated.

### Example 6

### Induction of Anti-Angiogenesis Factor TSP-1 in Mouse Stromal Cells from Xenografted H460 Tumors from Mice Treated with Compound a and Compound B In Vivo

Male BALBc/A nude mice (from Japan Crea Inc., Japan) implanted with H460 tumors were treated with either vehicle (0.5% HPMC) or CpdA (100 mg/kg) or Cpd B (40 mg/kg) by three times a week. Each group contains three mice. Tumor tissues were harvested 6 hours after the last administration at the end of week 1. The expression level of TSP-1 mRNA was detected by ABI7700 analyzer using probe/primer pre-mixture reagent (ABI, Cat# Mm01335418 ml) and TaqMan® Universal PCR Master Mix (ABI, Cat# 430-4437) as described in the ABI's protocol.

### Example 7

### Recombinant TSP-1 Potentiates Proapoptotic Effect of Taxol Toward Mouse Endothelial Cells In Vitro

Mouse endothelial MS-1 cells were seeded into 96 well-plate and incubated in 5% CO₂ incubator for 24 hours. Taxol at various concentrations was added into cell culture and 6 hours later media were replaced with fresh media containing recombinant TSP1 (10 ug/ml) but without taxol. After 72 hour incubation, the growth inhibitory effect was determined by crystal violet staining.

### Example 8

### Microarray Gene Expression Analysis of Human Cancer HCT15 Cancer Cells Treated with Compound A or Compound B In Vitro

Human colon cancer HCT15 cells were treated with Compound A or Compound B or Compound D for 24 hours in vitro. Total RNAs were extracted and RNA quality analysis was done using Agilent 2100 bioanalyzer and Agilent's RNA Labchip kits. RNAs were labeled with either Cy3 or Cy5 using Agilent's optimized labeling kits and hybridized to Human whole genome 44K Oligo Microarray (Agilent, Palo Alto, Calif.). Slides were scanned using DNA microarry scanner from Agilent and the raw data was extracted using Agilent's image analysis tool (feature extraction software). Normalization and statistical analysis were performed using GeneSpring software. Biological analysis was performed using Biointerpreter software.

### Example 9

### Induction of Transcription of MT3 in Human Cancer Cells Treated with Compound A, Compound B, Compound C, Compound D or SAHA In Vitro Analyzed by Real Time RT-PCR

Human cancer colon cancer HCT15 cells, leukemic Jurkat-T cells, and lymphoma RPMI-8226 cells were treated with various concentrations of Compound A, Compound B, or inactive analog of Compound A (Compound C), or Compound D, or SAHA for 24 hours in vitro. Total RNAs were extracted from cell pellets or from tumors using QiaShredder and RNeasy mini kit (Qiagen). 1 μg RNA was converted into cDNA using Expand RT enzyme (Roche) and Oligo(dT) primers (Invitrogen) in a 20 μl reaction volume. For quantitative real-time PCR, the primers used for MT3 were 5′CCC TGC GGA GTG TGA GAA GT 3′ and 5′TGC TTC TGC CTC AGC TGC CT 3′ and those for β-actin were 5′CTC TTC CAG CCT TCC TTC CT 3′ and 5′AGC ACT GTG TTG GCG TAC AG 3′. Reactions with either pair of primers included an annealing temperature of 63.4° C. All real-time PCR reactions were performed on the MasterCycler ep Realplex (Eppendorf) using FastStart SYBRGreen Master (Roche).

### Example 10

### Induction of Transcription of MT3 in Implanted H460 Tumor In Vivo in Mice Treated with Compound A Orally

Male BALBc/A nude mice (from Japan Crea Inc., Japan) implanted with H460 tumors were treated with either vehicle (0.5% HPMC) or 100 mg/kg Compound A (2HBr salt) as single administration. 6 hours or 24 hours post drug administration, mice were sacrificed and tumor excised and put in RNAlater (Ambion, Austin, Tex.) and stored at −70 C until RNAs were extracted using QiaShredder and RNeasy mini kit (Qiagen). For real time RT-PCR to determine MT3 transcription level, 1 μg RNA was converted into cDNA using Expand RT enzyme (Roche) and Oligo(dT) primers (Invitrogen) in a 20 μl reaction volume. For quantitative real-time PCR, the primers used for MT3 were 5′CCC TGC GGA GTG TGA GAA GT 3′ and 5′TGC TTC TGC CTC AGC TGC CT 3′ and those for β-actin were 5′CTC TTC CAG CCT TCC TTC CT 3′ and 5′AGC ACT GTG TTG GCG TAC AG 3′. Reactions with either pair of primers included an annealing temperature of 63.4° C. All real-time PCR reactions were performed on the MasterCycler ep Realplex (Eppendorf) using FastStart SYBRGreen Master (Roche).

### Example 11

### Generation of Human Colon Cancer HCT15 Clones which Overexpress MT3

To obtain clones overexpressing MT3, colon adenocarcinoma HCT15 cells (ATCC) were lipofectin-transfected for 6 h with a pCMV6-XL5 vector expressing MT3 (Origene) along with pcDNA3.1 plasmid to confer resistance to Geneticin (Gibco). Selection with 400 μM Geneticin was initiated after 48 h and allowed the formation of colonies. Individual, well isolated clones were picked up after 19 days of selection. Several independent clones were selected from separate plates. A control clone was obtained by transfecting HCT15 cells with pcDNA3.1 alone.

### Example 12

### Induction of Apoptosis in Human Cancer HCT15 Cells by Overexpressing MT3

Human colon cancer HCT15 clones which overexpresses MT3 were analyzed for induction of apoptosis by measuring the amount of cytoplasmic oligonucleosome release using “Cell Death ELISA Plus” kit (Roche, Cat#1774425). Typically, 2×10⁴ cells were seeded in each well of a 96-well plate and allowed to settle for one day. After a 16-hour long treatment with various concentrations of compounds, apoptosis was evaluated according to manufacturer's instructions.

### Example 13

### Inhibition of Anchorage-Independent Growth in Human HCT15 Cancer Cells by Overexpressing MT3

Cells from MT3 overexpressing stable clones or vector control were trypsinized and counted, then plated as a suspension in a soft agar layer (0.26% agar in 1× Iscove's supplemented with 20% FBS), in sandwich between two feeding layers (0.6% agar in 1× Iscove's plus 10% FBS). After two weeks colonies were counted manually.

### Example 14

### In Vitro Sensitivity of Human Cancer HCT15 Cells which Overexpresses MT3 Toward Taxane Compounds

MT3 overexpressing cells or vector control cells were assayed for their sensitivity to chemoagents by MTT. Cells were incubated with tested compounds in a 96-well format for 72 hours at 37° C. in 5% CO₂ incubator, then MTT (3-[4,5-dimethylthiazol-2-yl]-2,5 diphenyl tetrazolium bromide, Sigma) was added for 4 h and solubilized dye was subsequently quantified by OD_{(57-630 nm)}. Readings were converted to cell numbers according to a standard growth curve of the relevant cell line. The concentration which reduces cell numbers to 50% of that of solvent treated cells was defined as MTT IC₅₀.

### Example 15

### Potentiation of Antitumor Activity of Taxol with Oral Administration of Compound A, Compound B or SAHA In Vivo in Nude Mice Bearing Implanted Human Lung, Prostate, and Gastric Tumors

Antitumor studies were done using human H460 non-small cell lung tumor, Du145 prostate tumor, TSU-Pr1 prostate tumor, and AZ521 gastric tumor xenograft model in male BALBc/A nude mice (from Japan Crea Inc., Japan). Male nude mice were used at age 8-10 weeks. Human carcinoma cells were injected subcutaneously in the animal flank and allowed to form solid tumors. Tumor fragments (about 2 mm³ fragments) were then removed and implanted subcutaneously through a small surgical incision to the right flank of other animals. When the tumor sizes reached about 100 to 200 mm³, recipient animals were treated with vehicle (0.5% HPMC, hydroxypropoximethylellulose), Compound A (2HBr salt, dissolved in 0.1N HCl), Compound B (suspended in 0.5% HPMC, hydroxypropoximethylellulose), or SAHA by oral administration, or taxol by i.v. injection, or combination of taxol with oral administration of Compound A, or with Compound B, or with SAHA. Typically, taxol was administered once per week by iv injection on the first day of the week in the morning, while Compound A, Compound B or SAHA were administered 3 times weekly on day 1, day 3 and day 5. Tumor volumes and gross body weight of animals were monitored twice weekly for up to 2 weeks for AZ-521 and TSU-Pr1, or 4 weeks for H460 and Du145 xenografts. Each experimental group contained 6 animals.

### Example 16

### Potentiation of Antitumor Activity of Taxol with Compound a In Vivo in Nude Mice Bearing Implanted Human Lung Tumors by iv-iv Combination

Antitumor studies were done using human H460 non-small cell lung tumor xenograft model in male BALBc/A nude mice (from Japan Crea Inc., Japan). Male nude mice were used at age 8-10 weeks. Human carcinoma cells were injected subcutaneously in the animal flank and allowed to form solid tumors. Tumor fragments (about 2 mm³ fragments) were then removed and implanted subcutaneously to the right flank of other animals. When the tumor sizes reached about 100 to 200 mm³, recipient animals were treated by injection as a single shot with vehicle (2.5% DMSO, 7.5% Tween 80 in water), Compound A (2HBr salt, 40 mg/kg) or taxol (60 mg/kg), or combination of Compound A with taxol, by i.v. on day 1. Tumor volumes and gross body weight of animals were monitored twice weekly for up to 15 days. Each experimental group contained 6 animals.

### Example 17

### Potentiation of Antitumor Activity of Taxotere with Compound a In Vivo in Nude Mice Bearing Implanted Human Lung Tumors

Antitumor studies were done using human H460 non-small cell lung tumor xenograft model in male BALBc/A nude mice (from Japan Crea Inc., Japan). Male nude mice were used at age 8-10 weeks. Human carcinoma cells were injected subcutaneously in the animal flank and allowed to form solid tumors. Tumor fragments (about 2 mm² fragments) were then removed and implanted subcutaneously to the right flank of other animals. When the tumor sizes reached about 100 to 200 mm³, recipient animals were treated with vehicle (0.5% HPMC, hydroxypropoximethylellulose), Compound A (2HBr salt) by oral administration, or taxotere by i.v. injection, or combination of taxotere with oral administration of Compound A. Typically, taxotere was administered as a single dose by iv injection on day 1 (schedule A) or on day 8 (schedule B), while Compound A was administered 3 times weekly for 3 weeks. Tumor volumes and gross body weight of animals were monitored twice weekly for up to 3 weeks. Each experimental group contained 6 animals.

### Example 18

### Potentiation of Antitumor Activity of Taxol by Compound D In Vivo in Nude Mice Bearing Human AZ521 Gastric Tumors

Antitumor studies were done using human AZ521 gastric tumor xenograft model in male BALBc/A nude mice (from Japan Crea Inc., Japan). Male nude mice were used at age 8-10 weeks. Human carcinoma cells were injected subcutaneously in the animal flank and allowed to form solid tumors. Tumor fragments (about 2 mm² fragments) were then removed and implanted subcutaneously to the right flank of other animals. When the tumor sizes reached about 100 to 200 mm³, recipient animals were treated Compound D (40 mg/kg, suspended with 0.5% HPMC) by oral administration alone, or taxol by i.v. injection (20 mg/kg), or combination of taxol with oral administration of Compound D. Typically, taxol was administered once per week by iv injection on the first day as a single administration, while Compound D was administered once daily for 14 days. Tumor volumes and gross body weight of animals were monitored twice weekly for up to 2 weeks. Each experimental group contained 6 animals.

### Example 19

### Potentiation of Antitumor Activity of Taxol by Compound D, E, F, G, H with Taxol In Vivo in Nude Mice Bearing Human Prostate Du145 Tumors

Antitumor studies were done using human Du145 prostate tumor xenograft model in male BALBc/A nude mice (from Japan Crea Inc., Japan). Male nude mice were used at age 8-10 weeks. Human carcinoma cells were injected subcutaneously in the animal flank and allowed to form solid tumors. Tumor fragments (about 2 mm² fragments) were then removed and implanted subcutaneously to the right flank of other animals. When the tumor sizes reached about 100 to 200 mm³, recipient animals were treated with Compound D, Compound E, Compound F, Compound G or Compound H (suspended with 0.5% HPMC) by oral administration alone, or taxol by i.v. injection (60 mg/kg), or combination of taxol with oral administration of Compound D, Compound E, Compound F, Compound G, Compound H. Typically, taxol was administered once per week by iv injection on the first day as a single administration, while Compound D, Compound E, Compound F, Compound G, or Compound H were administered once daily for 14 days. Tumor volumes and gross body weight of animals were monitored twice weekly for up to 2 weeks. Each experimental group contained at least 6 animals.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for inhibiting abnormal cell growth and/or abnormal cell proliferation in a mammal, comprising administering to a mammal in need thereof an effective amount of a selective inhibitor of histone deacetylase (HDAC)1, HDAC2 and/or HDAC3 in combination with an effective amount of a compound that stabilizes microtubules.

2. The method according to claim 1, wherein the selective inhibitor of HDAC1, HDAC2 and/or HDAC 3 has a structure represented by Formula (I), Formula (II) or Formula (III) and N-oxides, hydrates, solvates, pharmaceutically acceptable salts, prodrugs and complexes thereof, and racemic and scalemic mixtures, diastereomers, enantiomers and tautomers thereof, wherein
Formula (I) has the structure 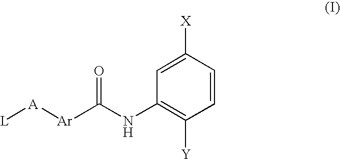
wherein
X is H, halo-, C₁-C₄-alkyl, C₁-C₄-alkoxy, —CH₂F, —CHF₂, —CF₃, aryl or heteroaryl, each of which is optionally substituted (preferably with one to three substituents independently selected from halo, —CN, —CH═N(OH), hydroxy, C₁-C₃-hydrocarbyl, —O—C₁-C₄alkyl, methoxy, or mono-, di-, or tri-halo substituted alkyl),
Y is —NH₂ or OH;
Ar is arylene or heteroarylene, each of which is optionally substituted;
A is selected from the group consisting of a covalent bond, M¹-L²-Ml, and L²-M²-L² wherein
L², at each occurrence, is independently selected from the group consisting of a chemical bond, C₀-C₄ hydrocarbyl, C₀-C₄-hydrocarbyl-(NH)-C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-SO—C₀-C₄-hydrocarbyl, C₀-C₄-hydrocarbyl-SO₂-C₀-C₄-hydrocarbyl, C₀-C₄-hydrocarbyl-NH—CO—C₀-C₄-hydrocarbyl, and C₀-C₄-hydrocarbyl-CO—NH—C₀-C₄-hydrocarbyl, provided that L² is not a chemical bond when X¹ is M¹-L²-M¹;
M¹, at each occurrence, is independently selected from the group consisting of —O—, —N(R⁷)—, —S—, —S(O)—, S(O)₂—, —S(O)₂N(R⁷)—, —N(R⁷)—S(O)₂—, —C(O)—, —C(O)—NH—, —NH—C(O)—, —NH—C(O)—O— and —O—C(O)—NH—, wherein R⁷ is selected from the group consisting of hydrogen, alkyl, aryl, aralkyl, acyl, heterocyclyl, and heteroaryl; and
M² is selected from the group consisting of M¹, heteroarylene, and heterocyclylene, either of which rings optionally is substituted; and
L is selected from the group consisting of H, cycloalkyl, aryl, heteroaryl, or heterocyclyl, each of which is optionally substituted and each of which is optionally fused to one or more aryl or heteroaryl rings, or to one or more saturated or partially unsaturated cycloalkyl or heterocyclic rings, each of which rings is optionally substituted;
Formula (II) has the structure: 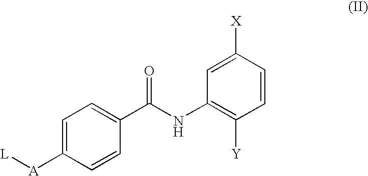
wherein
X is H, phenyl, thienyl, furanyl, pyridyl or pyrimidyl, each of which is optionally substituted;
Y is —NH₂;
A is —N(R⁷)—(CH₂)—; and
L is -heteroaryl-heteroaryl, -alkyl or heteroaryl, each of which is optionally substituted; wherein
R⁷ is selected from the group consisting of hydrogen, alkyl, aryl, aralkyl, acyl, heterocyclyl, and heteroaryl; and
Formula (III) has the structure: 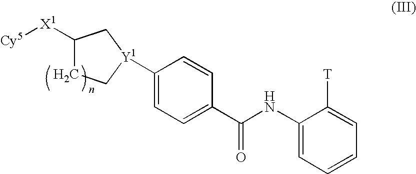
wherein
Cy⁵ is aryl, or heteroaryl, each of which is optionally substituted and wherein each of aryl and heteroaryl is optionally fused to one or more aryl or heteroaryl rings, or to one or more saturated or partially unsaturated cycloalkyl or heterocyclic rings, each of which rings is optionally substituted;
X¹ is selected from the group consisting of: a covalent bond, C₀-C₄-hydrocarbyl, C₀-C₄-hydrocarbyl-(CO)—C₀-C₄-hydrocarbyl, C₀-C₄-hydrocarbyl-N(R⁸)—C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-(SO)—C₀-C₄-hydrocarbyl, C₀-C₄-hydrocarbyl-(SO₂)-C₀-C₄-hydrocarbyl, C₀-C₄-hydrocarbyl-(NH)—(CO)—C₀-C₄-hydrocarbyl, C₀-C₄-hydrocarbyl -(CO)—(NH)—C₀-C₄-hydrocarbyl, —NH—CO—NH—, —NH—CS—NH—, —O—CO—O—, —O—CS—O—, —NH—C(NH)—NH—, —S(O)₂—N(R⁸)—, —N(R⁸)—S(O)₂—, —NH—C(O)—O—, and —O—C(O)—NH—;
wherein R⁸ is selected from the group consisting of hydrogen, C₁-C₅-alkyl, aryl, aralkyl, acyl, heterocyclyl, heteroaryl, SO₂-alkyl, SO₂-aryl, CO-alkyl, CO-aryl, CO—NH-alkyl, CO—NH-aryl, CO—O-alkyl and CO—O-aryl, each of which is optionally substituted;
n is 0 to 4;
Y¹ is N or CH; and
T is NH₂ or OH.

3. The method according to claim 1, wherein the selective inhibitor of HDAC1, HDAC2 and/or HDAC 3 has the structure

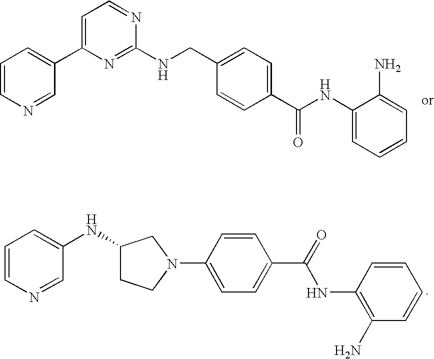

4. The method according to claim 1, wherein the compound that stabilizes microtubules is a taxane, an epothilone or an elpothilone analog.

5. The method according to claim 4, wherein the taxane is taxol or taxotere.

6. A method for inhibiting abnormal cell growth and/or abnormal cell proliferation in a mammal, comprising administering to a mammal in need thereof an effective amount of a selective inhibitor of histone deacetylase (HDAC)1, and/or HDAC2 in combination with an effective amount of a compound that stabilizes microtubules.

7. The method according to claim 6, wherein the selective inhibitor of HDAC1 and/or HDAC2 has a structure represented by Formula (IV), Formula (IVa) or Formula (V) and N-oxides, hydrates, solvates, pharmaceutically acceptable salts, prodrugs and complexes thereof, and racemic and scalemic mixtures, diastereomers, enantiomers and tautomers thereof, wherein
Formula (IV) has the structure. 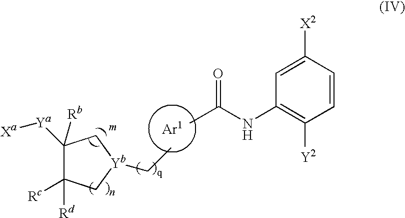
wherein
X² is aryl, cycloalkyl, heteroaryl or heterocyclyl, each of which is optionally substituted;
Ar¹ is aryl, heteroaryl, cycloalkyl or heterocyclyl, each of which is optionally substituted;
R^{a} is H or an optional substituent, preferably halo;
R^{b}, R^{c} and R^{d} are each independently hydrogen, C₁-C₈ alkyl, aryl, heteroaryl, cycloalkyl, heterocyclyl or halo; or
R^{b} and R^{c} together with the atoms to which they are bonded, optionally form a 5- or 6-membered cycloalkyl or heterocycloalkyl having 1 or 2 annular heteroatoms; each of which is optionally substituted with from 1 to 3 substituents;
Y² is —NH₂ or —OH;
Y^{b} is —N— or —CH—;
Y^{a} is a direct
bond, —O—, —N(R³⁴)—, —C(O)—, —OC(O)—, —C(O)O—, —N(R³⁴)—C(O)—, —C(O)—N(R³⁴)—, —N(R³⁴)—C(S) —, —C(S)—N(R³⁴)—, —N(R³⁴)—C(O)—N(R³⁵)—, —N(R³⁴)—C(NR³⁴)—N(R³⁵)—, —N(R³⁴)—C(NR³⁵), —C(NR³⁵)—N(R³⁴)—, —N(R³⁴)—C(S)—N(R³⁵)—, —N(R³⁴)—C(O)—O—, —O—C(O)—N(R³⁴)—, —N(R³⁴)—C(S)O—, —O—-C(S)—N(R³⁵)—, —S(O)₀₋₂, —SO₂N(R³⁵)—, —N(R³⁵)—SO₂—, N(R³⁴)—S(O)₂—N(R³⁵)—, —O—C₁-C₃alkyl-, —N(R³⁴)—C₁-C₃alkyl-, —C(O)—C₁-C₃alkyl- or —O—C(O)—C₁-C₃alkyl-;
X^{a} is C₁-C₈alkyl-, C₁-C₈alkenyl-, C₁-C₈alkynyl-, C₀-C₃alkyl-C₁-C₈alkenyl-C₀-C₃alkyl-, C₀-C₃alkyl-C₁-C₈alkynyl-C₀-C₃alkyl-, C₁-C₃alkyl-O—C₁-C₃alkyl-, HO—C₁-C₃alkyl-, C₁-C₄alkyl-N(R³⁴)—C₀-C₃alkyl-, N(R³⁴)(R³⁵)—C₀-C₃alkyl-, C₁-C₃alkyl-S(O)₀₋₂—C₁-C₃alkyl-, CF₃—C₀-C₃alkyl-, CF₂H-C₀-C₃alkyl-, C₁-C₈heteroalkyl-, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, aryl-C₀-C₂alkyl-heterocyclyl-C₀-C₂alkyl-, heteroaryl-C₀-C₂alkyl-heterocyclyl-C₀-C₂alkyl-, N(R³⁴)(R³⁵)-heterocyclyl-C₀-C₃alkyl-, heteroaryl-C₀-C₃alkyl-heterocyclyl- or C₁-C₄alkyl-CH(N(R³⁴)(R³⁵))—C(O)—N(R³⁴)-aryl-, wherein the aryl, cycloalkyl, heteroaryl and heterocycyl are optionally substituted with from 1 to 3 independently selected substituents;
or
X^{a}-Y^{a}- is selected from the group consisting of H—, halo-, HO—, HS—, HC(O)—, HOC(O)—, C₁-C₄alkyl-, H₂N—, (R³⁴)(R³⁵)N—, C₁-C₄alkyl-NH—, (C₁-C₄alkyl)₂-N—, HC(O)N(R³⁴)—, (R³⁴)(R³⁵)N—S(O)₂—N(R³⁶)—, (R³⁴)(R³⁵)N—C(O)—, H₂N—C(O)—, HC(S)N(R³⁴)—, (R³⁴)(R³⁵)N—C(S)—, H₂N—C(S)—, (R³⁴)(R³⁵)N—C(O)—O—, (R³⁴)(R³⁵)N—C(S)—O—, (R³⁴)(R³⁵)N—C(O)—N(R³⁶)—, (C₁-C₃alkylN)₂—C═N—, (R³⁴)(R³⁵)N—C(NR³⁷)—N(R³⁶)—, (R³⁴)(R³⁵)N—C(NR³⁶)—, cycloalkyl-C₀-C₂alkyl-C(NR³⁶)—, heterocyclyl-C₀-C₂alkyl-C(NR³⁶)—, aryl-C₀-C₂alkyl-C(NR³⁶)—, heteroaryl-C₀-C₂alkyl-C(NR³⁶)—, C₀-C₃alkyl-C(NR³⁶)—, C₁-C₄alkyl-S(O)₂—N(R³⁶)—, CF₃—C₀-C₄alkyl-S(O)₂—N(R³⁶)—, CF₃—C₀-C₄alkyl-C(O)—N(R³⁶)—, aryl-C₀-C₄alkyl-S(O)₂—N(R³⁶)—, heteroaryl-C₀-C₄alkyl-S(O)₂—N(R³)—, cycloalkyl-C₀-C₄alkyl-S(O)₂—N(R³⁶)—, heterocyclyl-C₀-C₄allyl-O S(O)₂—N(R³⁶)—, C₁-C₄alkyl-O—C(O)—NH—, C₁-C₄alkyl-O—C(O)—N(H)—C₁-C₄alkyl-, C₁-C₄alkyl-N(H)—C(O)—N(H)—, C₁-C₄alkyl-NH—C(O)—O—, C₁-C₄alkyl-C(O)—N(H)—, C₁-C₄alkyl-O—C(S)—N(H)—, C₁-C₄alkyl-N(H)—C(S)—N(H)—, C₁-C₄alkyl-N(H)—C(S)—O—, C₁-C₄alkyl-C(S)—N(H)—, Me-C(O)—O—, Me-C(O)—N(H)—, aryl-C₀-C₄alkyl-O—C(O)—N(H)—, aryl-C₀-C₄alkyl-O—C(O)—N(C₁-C₄alkyl)-, aryl-C₀-C₄alkyl-C(O)—N(H)—, heteroaryl-C₀-C₄alkyl-O—C(O)—N(H)—, heteroaryl-C₀-C₄alkyl-O—C(O)—N(C₁-C₄alkyl)-, heteroaryl-C₀-C₄alkyl-C(O)—N(H)—, aryl-C₀-C₄alkyl-N(H)—C(O)—O—, heteroaryl-C₀-C₄alkyl-N(H)—C(O)—O—, heterocyclyl-C₀-C₄alkyl-O—C(O)—N(H)—, heterocyclyl-C₀-C₄alkyl-O—C(O)—N(C₁-C₄alkyl)-, heterocyclyl-C₀-C₄alkyl-C(O)—N(H)—, cycloalkyl-C₀-C₄alkyl-O—C(O)—N(H)—, cycloalkyl-C₀-C₄alkyl-O—C(O)—N(C₁-C₄alkyl)-, cycloalkyl-C₀-C₄alkyl-C(O)—N(H)—, heterocyclyl-C₀-C₄alkyl-N(H)—C(O)—O—, cycloalkyl-C₀-C₄alkyl-N(H)—C(O)—O—, heterocyclyl-C₀-C₄alkyl-C(O)—N(H)—, aryl-C₀-C₄alkyl-N(H)—C(O)—N(H)—, aryl-C₀-C₄alkyl-N(H)—, aryl-C₀-C₄alkyl-O—, aryl-C₀-C₄alkyl-S(O)₀₋₂—, heteroaryl-C₀-C₄alkyl-N(H)—C(O)—N(H)—, heteroaryl-C₀-C₄alkyl-N(H)—, heteroaryl-C₀-C₄alkyl-O—, heteroaryl-C₀-C₄alkyl-S(O)₀₋₂—, heterocyclyl-C₀-C₄alkyl-N(H)—C(O)—N(H)—, heterocyclyl-C₀-C₄alkyl-N(H)—, heterocyclyl-C₀-C₄alkyl-O—, heterocyclyl-C₀-C₄alkyl-S(O)₀₋₂—, cycloalkyl-C₀-C₄alkyl-N(H)—C(O)—N(H)—, cycloalkyl-C₀-C₄alkyl-N(H)—, cycloalkyl-C₀-C₄alkyl-O—, cycloalkyl-C₀-C₄alkyl-S(O)₀₋₂—, aryl-C₀-C₄alkyl-C(S)—N(H)—, heteroaryl-C₀-C₄alkyl-C(S)—N(H)—, aryl-C₀-C₄alkyl-O—C(S)—N(H)—, heteroaryl-C₀-C₄alkyl-O—C(S)—N(H)—, aryl-C₀-C₄alkyl-N(H)—C(S)—O—, heteroaryl-C₀-C₄alkyl-N(H)—C(S)—O—, heterocyclyl-C₀-C₄alkyl-C(S)—N(H)—, cycloalkyl-C₀-C₄alkyl-C(S)—N(H)—, heterocyclyl-C₀-C₄alkyl-O—C(S)—N(H)—, cycloalkyl-C₀-C₄alkyl-O—C(S)—N(H)—, heterocyclyl-C₀-C₄alkyl-N(H)—C(S)—O—, cycloalkyl-C₀-C₄alkyl-N(H)—C(S)—O—, heterocyclyl-C₀-C₄alkyl-C(S)—N(H)—, aryl-C₀-C₄alkyl-N(H)—C(S)—NH—, heteroaryl-C₀-C₄alkyl-N(H)—C(S)—N(H)—, heterocyclyl-C₀-C₄alkyl-N(H)—C(S)—N(H)—, cycloalkyl-C₀-C₄alkyl-N(H)—C(S)—N(H)—, C₁-C₄alkyl-O—C₁-C₄alkyl-C(O)—N(H)—, C₁-C₄alkyl-O—C₂-C₄alkyl-O—C(O)—N(H)—, C₁-C₄alkyl-O—C₂-C₄alkyl-N(H)—C(O)—N(H)—, C₁-C₄alkyl-O—C₂-C₄alkyl-N(H)—, C₁-C₄alkyl-O—C₂-C₄alkyl-O—, C₁-C₄alkyl-O—C₂-C₄alkyl-N(H)—C(O)—O—, HO—C₁-C₄alkyl-C(O)—N(H)—, HO—C₁-C₄alkyl-N(H)—, HO—C₁-C₄alkyl-N(R³)—, HO—C₁-C₄alkyl-O—, HO—C₁-C₄alkyl-S(O)₀₋₂—, HO—C₂-C₄alkyl-O—C(O)—N(H)—, HO—C₂-C₄alkyl-N(H)—C(O)—N(H)—, HO—C₂-C₄alkyl-N(H)—C(O)—O—, C₁-C₄alkyl-O—C₁-C₄alkyl-C(S)—N(H)—, C₁-C₄alkyl-O—C₂-C₄alkyl-O—C(S)—N(H)—, C₁-C₄alkyl-O—C₂-C₄alkyl-N(H)C(S)—N(H)—, C₁-C₄alkyl-O—C₂-C₄alkyl-N(H)—C(S)—O—, HO—C₂-C₄alkyl-O—C(S)—N(H)—, HO—C₂-C₄alkyl-N(H)—C(S)—N(H)—, HO—C₂-C₄alkyl-N(H)—C(S)—O—, (C₁-C₄alkyl)₂N—C₁-C₄alkyl-C(O)—N(H)—, (CO—C₄alkyl)-O—C₁-C₄alkyl-C(O)—N(H)—, (CO—C₄alkyl)-O—C₁-C₄alkyl-C(S)—N(H)—, (CO—C₄alkyl)-O—C₁-C₄alkyl-C(O)—O—, (CO—C₄alkyl)-O—C₂-C₄alkyl-N(H)—C(O)—N(H)—, (CO—C₄alkyl)-O—C₂-C₄alkyl-O—C(O)—N(H)—, (CO—C₄alkyl)-O—C₂-C₄alkyl-N(H)—C(NH)—N(H)—, (CO—C₄alkyl)-O—C₂-C₄alkyl-N(H)—C(O)—, (C₁-C₄alkyl)₂N—C₂-C₄alkyl-O—C(O)—N(H)—, (C₁-C₄alkyl)₂N—C₂-C₄alkyl-N(H)—, (C₁-C₄alkyl)₂N—C₂-C₄alkyl-O—, (C₁-C₄alkyl)₂N—C₂-C₄alkyl-S(O)₀₋₂—, (C₁-C₄alkyl)₂N—C₂-C₄alkyl-N(H)—C(O)—N(H)—, (C₁-C₄alkyl)₂N—C₂-C₄alkyl-N(H)—C(O)—O—, (C₁-C₄alkyl)₂N—C₁-C₄alkyl-C(S)—N(H)—, (C₁-C₄alkyl)₂N—C₂-C₄alkyl-N(H)—C(S)—N(H)—, (C₁-C₄alkyl)₂N—C₂-C₄alkyl-N(H)—C(S)—O—, (C₁-C₄alkyl)-O—C(O)C₁-C₈alkyl-C(O)—(H)—, HO—C(O)C₁-C₈alkyl-C(O)—N(H)—, HO—NH—C(O)C₁-C₈alkyl-C(O)—N(H)—, CF₂H—C₀-C₄alkyl-C(O)—N(H)—, CF₃—C₀-C₄alkyl-C(O)—N(H)—, CF₃—C₀-C₄alkyl-N(H)—, CF₃—C₀-C₄alkyl-N(R³)—, CF₃—C₀-C₄alkyl-O—, CF₃—C₀-C₄alkyl-S(O)₀₋₂—, CF₃—C₀-C₄alkyl-O—C(O)—N(H)—, CF₃—C₀-C₄alkyl-N(H)C(O)—N(H)—, CF₃—C₀-C₄alkyl-N(H)—C(O)—O—, CF₃—C₀-C₄alkyl-O—C(S)—N(H)—, CF₃—C₀-C₄alkyl-N(H)—C(S)—N(H)—, CF₃—C₀-C₄alkyl-N(H)—C(S)—O—, CF₃-C₀-C₄alkyl-C(S)—N(H)—, CF₂H—C₀-C₄alkyl-N(H)—, CF₂H—C₀-C₄alkyl-O—, CF₂H—C₀-C₄alkyl-S(O)₀₋₂—, CF₂H—C₀-C₄alkyl-O—C(O)—N(H)—, CF₂H—C₀-C₄alkyl-N(H)C(O)—N(H)—, CF₂H—C₀-C₄alkyl-N(H)—C(O)—O—, CF₂H—C₀-C₄alkyl-O—C(S)—N(H)—, CF₂H—C₀-C₄alkyl-N(H)—C(S)—N(H)—, CF₂H—C₀-C₄alkyl-N(H)—C(S)—O—, CF₂H—C₀-C₄alkyl-C(S)—N(H)—, (H)(R³⁴)N—C₁-C₃alkyl-, (H)(R³⁴)N—C₁-C₃alkyl-, HO—C₁-C₃alkyl-, (H)(R³⁴)N—S(O)₂—N(R³⁵)—, (H)(R³⁵)N—S(O)₂—, (H)(R³⁴)N—C(S)—O—, (H)(R³⁴)N—C(O)—O—, (H)(R³⁴)N—C(S)—N(R³⁵)—, (H)(R³⁴)N—C(NR³⁵)—, (H)(R³⁴)N—C(NR³⁴)—N(R³⁸)—, (H)(R³⁴)N—C(O)—N(R³⁵)—, HO—C(O)—C₁-C₃alkyl-, C₁-C₄alkyl-S(O)₂—NH— and ((R³⁴)(R³⁵)N)₂—C═N—;
m and n are independently 0, 1, 2 or 3;
q is 0, 1 or 2; and
R³⁴, R³⁵, R³⁶ and R³⁷ are each independently selected from the group consisting of hydrogen, cyano, oxo, hydroxyl, —C₁-C₈alkyl, C₁-C₈heteroalkyl, C₁-C₈alkenyl, carboxamido, C₁-C₃alkyl-carboxamido-, carboxamido-C₁-C₃alkyl-, amidino, C₂-C₈hydroxyalkyl, C₁-C₃alkylaryl-, aryl-C₁-C₃alkyl-, C₁-C₃alkylheteroaryl-, heteroaryl-C₁-C₃alkyl-, C₁-C₃alkylheterocyclyl-, heterocyclyl-C₁-C₃alkyl-, C₁-C₃alkylcycloalkyl-, cycloalkyl-C₁-C₃alkyl-, C₂-C₈alkoxy-, C₂-C₈alkoxy-C₁-C₄alkyl-, C₁-C₈alkoxycarbonyl-, aryloxycarbonyl-, aryl-C₁-C₃alkoxycarbonyl-, heteroaryloxycarbonyl-, heteroaryl-C₁-C₃alkoxycarbonyl-, C₁-C₈acyl, C₀-C₈alkyl-carbonyl-, aryl-C₀-C₈alkyl-carbonyl-, heteroaryl-C₀-C₈alkyl-carbonyl-, cycloalkyl-C₀-C₈alkyl-carbonyl-, C₀-C₈alkyl-N(H)-carbonyl-, aryl-C₀-C₈alkyl-N(H)-carbonyl-, heteroaryl-C₀-C₈alkyl-N(H)-carbonyl-, cycloalkyl-C₀-C₈alkyl-N(H)-carbonyl-, C₀-C₈alkyl-O-carbonyl-, aryl-C₀-C₈alkyl-O-carbonyl-, heteroaryl-C₀-C₈alkyl-O-carbonyl-, cycloalkyl-C₀-C₈alkyl-O-carbonyl-, C₁-C₈ alkylsulfonyl-, arylalkylsulfonyl-, arylsulfonyl-, heteroarylalkylsulfonyl-, heteroarylsulfonyl-, C₁-C₈alkyl-N(H)-sulfonyl-, arylalkyl-N(H)-sulfonyl-, aryl-N(H)-sulfonyl-, heteroarylalkyl-N(H)-sulfonyl-, heteroaryl-N(H)-sulfonyl, aroyl, aryl, cycloalkyl, heterocyclyl, heteroaryl, aryl-C₁-C₃alkyl-, cycloalkyl-C₁-C₃alkyl-, heterocyclyl-C₁-C₃ alkyl-, heteroaryl-C₁-C₃ alkyl-, and a protecting group, wherein each of the foregoing is further optionally substituted with one more moieties; or
R³⁴ and R³⁵ taken together with the N to which they are attached form a heterocyclyl or heteroaryl, each of which is optionally substituted with from 1 to 3 substituents, wherein the heterocyclyl may also be bridged (forming a bicyclic moiety with a methylene, ethylene or propylene bridge),
provided that 1) when Y^{b} is N, then m is not 0 if Y^{a} is bound to the ring comprising Y, via a N, S or O in Y^{a}, or 2) when m and n are both 0 then Y^{b} is —CH—;
Formula (IVa) has the structure: 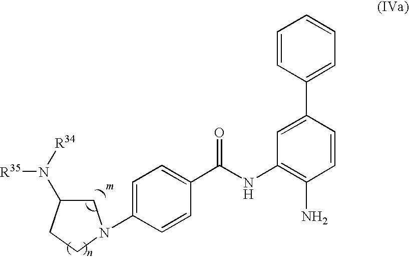
wherein m, n, R³⁴ and R³⁵ are as defined for formula (IV); and
Formula (V) has the structure: 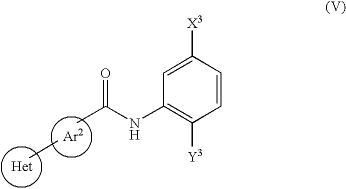
wherein
X³ is aryl, cycloalkyl, heteroaryl or heterocyclyl, each of which is optionally substituted;
Y³ is —NH₂ or —OH;
Ar² is optionally substituted aryl or optionally substituted heteroaryl; and
Het is an optionally substituted heterocyclyl.

8. The method according to claim 6, wherein the selective inhibitor of HDAC1 and/or HDAC2 has the structure

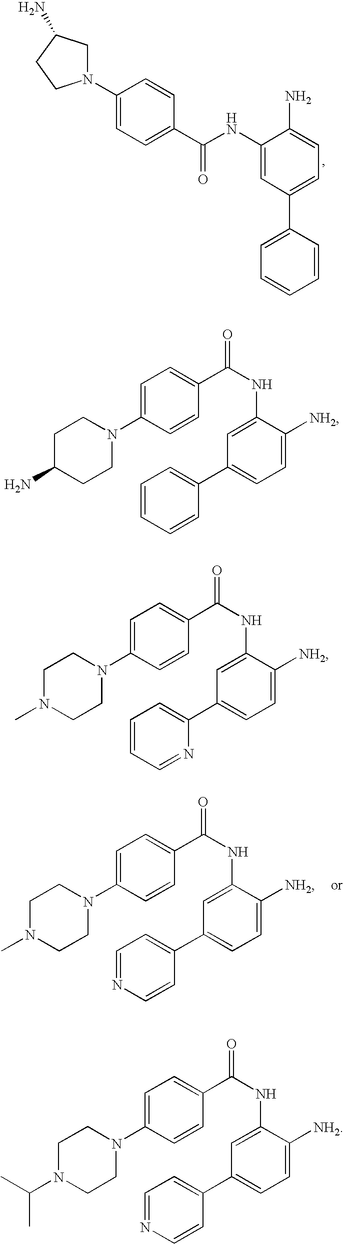

9. The method according to claim 6, wherein the compound that stabilizes microtubules is a taxane, an epothilone or an elpothilone analog.

10. The method according to claim 9, wherein the taxane is taxol or taxotere.

11. A method for inhibiting abnormal cell growth and/or abnormal cell proliferation in a mammal, comprising up-regulating the expression of metalothionene 3 (MT3) in the cells and/or up-regulating the expression of thrombospondin-1 (TSP1) in the cells, in combination with administering a compound that stabilizes microtubules.

12. The method according to claim 11, wherein the compound that stabilizes microtubules is a taxane, an epothilone or an elpothilone analog.

13. The method according to claim 12, wherein the taxane is taxol or taxotere.

14. A method for inhibiting abnormal cell growth and/or abnormal cell proliferation in a mammal, the method comprising administering to a mammal in need thereof an agonist of TSP1 receptor in combination with a compound that stabilizes microtubules.

15. The method according to claim 14, wherein the compound that stabilizes microtubules is a taxane, an epothilone or an elpothilone analog.

16. The method according to claim 15, wherein the taxane is taxol or taxotere.

17. A method for inhibiting abnormal cell growth and/or abnormal cell proliferation in a mammal, the method comprising up-regulating the expression of thrombospondin-1 (TSP1) in the cell, in combination with administering a compound that stabilizes microtubules.

18. The method according to claim 17, wherein the compound that stabilizes microtubules is a taxane, an epothilone or an elpothilone analog.

19. The method according to claim 18, wherein the taxane is taxol or taxotere.

20. A method for inhibiting abnormal cell growth and/or abnormal cell proliferation in a mammal, comprising up-regulating the expression of metalothionene 3 (MT3) in the cell and/or up-regulating the expression of thrombospondin-1 (TSP1) in the cell, in combination with administering a compound that stabilizes microtubules.

21. The method according to claim 20, wherein the compound that stabilizes microtubules is a taxane, an epothilone or an elpothilone analog.

22. The method according to claim 21, wherein the taxane is taxol or taxotere.

23. A method for inhibiting angiogenesis in a mammal, comprising administering an effective amount of a selective inhibitor of histone deacetylase (HDAC)1, HDAC2 and/or HDAC3.

24. The method according to claim 23 wherein the selective inhibitor of HDAC1, HDAC2 and/or HDAC 3 has a structure represented by Formula (I), Formula (II) or Formula (III) and N-oxides, hydrates, solvates, pharmaceutically acceptable salts, prodrugs and complexes thereof, and racemic and scalemic mixtures, diastereomers, enantiomers and tautomers thereof, wherein
Formula (I) has the structure 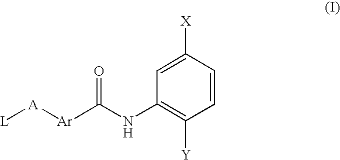
wherein
X is H, halo-, C₁-C₄-alkyl, C₁-C₄-alkoxy, —CH₂F, —CHF₂, —CF₃, aryl or heteroaryl, each of which is optionally substituted (preferably with one to three substituents independently selected from halo, —CN, —CH═N(OH), hydroxy, C₁-C₃-hydrocarbyl, —O—C₁-C₄alkyl, methoxy, or mono-, di-, or tri-halo substituted alkyl),
Y is —NH₂ or OH;
Ar is arylene or heteroarylene, each of which is optionally substituted;
A is selected from the group consisting of a covalent bond, M¹-L²-M¹, and L²-M²-L² wherein
L², at each occurrence, is independently selected from the group consisting of a chemical bond, C₀-C₄ hydrocarbyl, C₀-C₄-hydrocarbyl-(NH)—C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-SO—C₀-C₄-hydrocarbyl, C₀-C₄-hydrocarbyl-SO₂—C₀-C₄-hydrocarbyl, C₀-C₄-hydrocarbyl-NH—C₀-C₀-C₄-hydrocarbyl, and C₀-C₄-hydrocarbyl-CO—NH—C₀-C₄-hydrocarbyl, provided that L² is not a chemical bond when X¹ is M¹-L²-M¹;
M¹, at each occurrence, is independently selected from the group consisting of —O—, —N(R⁷)—, —S—, —S(O)—, S(O)₂—, —S(O)₂N(R⁷)—, —N(R⁷)—S(O)₂—, —C(O)—, —C(O)—NH—, —NH—C(O)—, —NH—C(O)—O— and —O—C(O)—NH—, wherein R⁷ is selected from the group consisting of hydrogen, alkyl, aryl, aralkyl, acyl, heterocyclyl, and heteroaryl; and
M² is selected from the group consisting of M¹, heteroarylene, and heterocyclylene, either of which rings optionally is substituted; and
L is selected from the group consisting of H, cycloalkyl, aryl, heteroaryl, or heterocyclyl, each of which is optionally substituted and each of which is optionally fused to one or more aryl or heteroaryl rings, or to one or more saturated or partially unsaturated cycloalkyl or heterocyclic rings, each of which rings is optionally substituted;
Formula (II) has the structure: 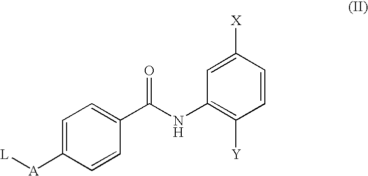
wherein
X is H, phenyl, thienyl, furanyl, pyridyl or pyrimidyl, each of which is optionally substituted;
Y is —NH₂;
A is —N(R⁷)—(CH₂)—; and
L is -heteroaryl-heteroaryl, -alkyl or heteroaryl, each of which is optionally substituted; wherein
R⁷ is selected from the group consisting of hydrogen, alkyl, aryl, aralkyl, acyl, heterocyclyl, and heteroaryl; and
Formula (III) has the structure: 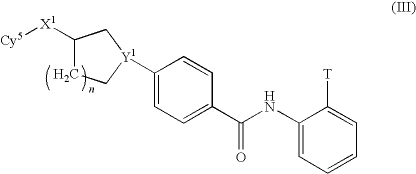
wherein
Cy⁵ is aryl, or heteroaryl, each of which is optionally substituted and wherein each of aryl and heteroaryl is optionally fused to one or more aryl or heteroaryl rings, or to one or more saturated or partially unsaturated cycloalkyl or heterocyclic rings, each of which rings is optionally substituted;
X¹ is selected from the group consisting of: a covalent bond, C₀-C₄-hydrocarbyl, C₀-C₄-hydrocarbyl-(CO)—C₀-C₄-hydrocarbyl, C₀-C₄-hydrocarbyl-N(R⁸)—C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-(SO)—C₀-C₄-hydrocarbyl, C₀-C₄-hydrocarbyl-(SO₂)-C₀-C₄-hydrocarbyl, C₀-C₄-hydrocarbyl-(NH)—(CO)—C₀-C₄-hydrocarbyl, C₀-C₄-hydrocarbyl -(CO)—(NH)—C₀-C₄-hydrocarbyl, —NH—CO—NH—, —NH—CS—NH—, —O—CO—O—, —O—CS—O—, —NH—C(NH)—NH—, —S(O)₂—N(R⁸)—, —N(R⁸)—S(O)₂—, —NH—C(O)—O—, and —O—C(O)—NH—;
wherein R⁸ is selected from the group consisting of hydrogen, C₁-C₈-alkyl, aryl, aralkyl, acyl, heterocyclyl, heteroaryl, SO₂-alkyl, SO₂-aryl, CO-alkyl, CO-aryl, CO—NH-alkyl, CO—NH-aryl, CO—O-alkyl and CO—O-aryl, each of which is optionally substituted;
n is 0 to 4;
Y¹ is N or CH; and
T is NH₂ or OH.

25. The method according to claim 23, wherein the selective inhibitor of HDAC1, HDAC2 and/or HDAC 3 has the structure

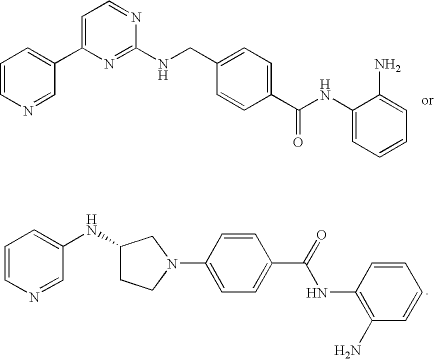

26. The method according to claim 23, wherein the compound that stabilizes microtubules is a taxane, an epothilone or an elpothilone analog.

27. The method according to claim 26, wherein the taxane is taxol or taxotere.

28. A method for inducing expression of an anti-angiogenesis factor in a cell, the method comprising administering to the cell a selective inhibitor of histone deacetylase (HDAC)1, HDAC2 and/or HDAC3.

29. The method according to claim 28, wherein the selective inhibitor of HDAC1, HDAC2 and/or HDAC 3 has a structure represented by Formula (I), Formula (II) or Formula (III) and N-oxides, hydrates, solvates, pharmaceutically acceptable salts, prodrugs and complexes thereof, and racemic and scalemic mixtures, diastereomers, enantiomers and tautomers thereof, wherein
Formula (I) has the structure 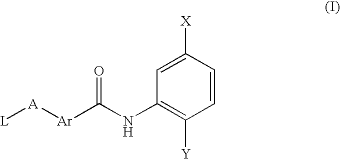
wherein
X is H, halo-, C₁-C₄-alkyl, C₁-C₄-alkoxy, —CH₂F, —CHF₂, —CF₃, aryl or heteroaryl, each of which is optionally substituted (preferably with one to three substituents independently selected from halo, —CN, —CH═N(OH), hydroxy, C₁-C₃-hydrocarbyl, —O—C₁-C₄alkyl, methoxy, or mono-, di-, or tri-halo substituted alkyl),
Y is —NH₂ or OH;
Ar is arylene or heteroarylene, each of which is optionally substituted;
A is selected from the group consisting of a covalent bond, M¹-L²-Ml, and L²-M²-L² wherein
L², at each occurrence, is independently selected from the group consisting of a chemical bond, C₀-C₄ hydrocarbyl, C₀-C₄-hydrocarbyl-(NH)—C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-SO—C₀-C₄-hydrocarbyl, C₀-C₄-hydrocarbyl-SO₂—C₀-C₄-hydrocarbyl, C₀-C₄-hydrocarbyl-NH—CO—C₀-C₄-hydrocarbyl, and C₀-C₄-hydrocarbyl-CO—NH—C₀-C₄-hydrocarbyl, provided that L² is not a chemical bond when X¹ is M¹-L¹-M¹;
M¹, at each occurrence, is independently selected from the group consisting of —O—, —N(R⁷)—, —S—, —S(O)—, S(O)₂—, —S(O)₂N(R⁷)—, —N(R⁷)—S(O)₂—, —C(O)—, —C(O)—NH—, —NH—C(O)—, —NH—C(O)—O— and —O—C(O)—NH—, wherein R⁷ is selected from the group consisting of hydrogen, alkyl, aryl, aralkyl, acyl, heterocyclyl, and heteroaryl; and
M² is selected from the group consisting of M¹, heteroarylene, and heterocyclylene, either of which rings optionally is substituted; and
L is selected from the group consisting of H, cycloalkyl, aryl, heteroaryl, or heterocyclyl, each of which is optionally substituted and each of which is optionally fused to one or more aryl or heteroaryl rings, or to one or more saturated or partially unsaturated cycloalkyl or heterocyclic rings, each of which rings is optionally substituted;
Formula (II) has the structure: 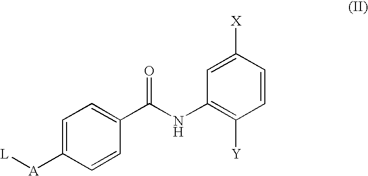
wherein
X is H, phenyl, thienyl, furanyl, pyridyl or pyrimidyl, each of which is optionally substituted;
Y is —NH₂;
A is —N(R⁷)—(CH₂)—; and
L is -heteroaryl-heteroaryl, -alkyl or heteroaryl, each of which is optionally substituted; wherein R⁷ is selected from the group consisting of hydrogen, alkyl, aryl, aralkyl, acyl, heterocyclyl, and heteroaryl; and
Formula (III) has the structure: 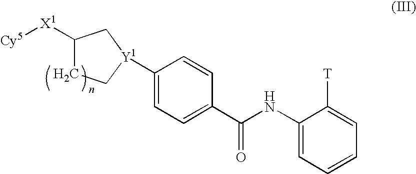
wherein
Cy⁵ is aryl, or heteroaryl, each of which is optionally substituted and wherein each of aryl and heteroaryl is optionally fused to one or more aryl or heteroaryl rings, or to one or more saturated or partially unsaturated cycloalkyl or heterocyclic rings, each of which rings is optionally substituted;
X¹ is selected from the group consisting of: a covalent bond, C₀-C₄-hydrocarbyl, C₀-C₄-hydrocarbyl-(CO)—C₀-C₄-hydrocarbyl, C₀-C₄-hydrocarbyl-N(R⁸)—C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-(SO)—C₀-C₄-hydrocarbyl, C₀-C₄-hydrocarbyl-(SO₂)-C₀-C₄-hydrocarbyl, C₀-C₄-hydrocarbyl-(NH)—(CO)—C₀-C₄-hydrocarbyl, C₀-C₄-hydrocarbyl -(CO)—(NH)—C₀-C₄-hydrocarbyl, —NH—CO—NH—, —NH—CS—NH—, —O—CO—O—, —O—CS—O—, —NH—C(NH)—NH—, —S(O)₂—N(R⁸)—, —N(R⁸)—S(O)₂—, —NH—C(O)—O—, and —O—C(O)—NH—;
wherein R⁸ is selected from the group consisting of hydrogen, C₁-C₅-alkyl, aryl, aralkyl, acyl, heterocyclyl, heteroaryl, SO₂-alkyl, SO₂-aryl, CO-alkyl, CO-aryl, CO—NH-alkyl, CO—NH-aryl, CO—O-alkyl and CO—O-aryl, each of which is optionally substituted;
n is 0 to 4;
Y¹ is N or CH; and
T is NH₂ or OH.

30. The method according to claim 28, wherein the selective inhibitor of HDAC1, HDAC2 and/or HDAC 3 has the structure

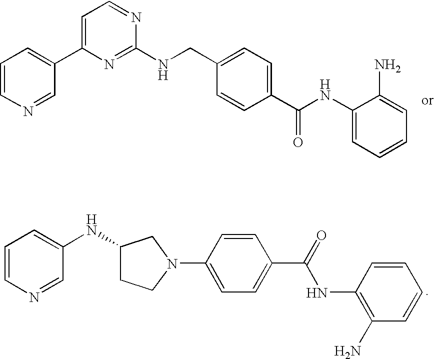

31. The method according to claim 28, wherein the compound that stabilizes microtubules is a taxane, an epothilone or an elpothilone analog.

32. The method according to claim 31, wherein the taxane is taxol or taxotere.

33. A method for inhibiting expression of an angiogenesis factor in a cell, the method comprising administering to the cell a selective inhibitor of histone deacetylase (HDAC)1, HDAC2 and/or HDAC3.

34. The method according to claim 33, wherein the selective inhibitor of HDAC1, HDAC2 and/or HDAC 3 has a structure represented by Formula (I), Formula (II) or Formula (III) and N-oxides, hydrates, solvates, pharmaceutically acceptable salts, prodrugs and complexes thereof, and racemic and scalemic mixtures, diastereomers, enantiomers and tautomers thereof, wherein
Formula (I) has the structure 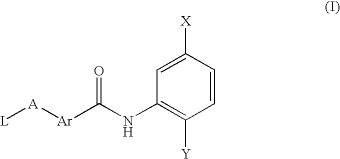
wherein
X is H, halo-, C₁-C₄-alkyl, C₁-C₄-alkoxy, —CH₂F, —CHF₂, —CF₃, aryl or heteroaryl, each of which is optionally substituted (preferably with one to three substituents independently selected from halo, —CN, —CH═N(OH), hydroxy, C₁-C₃-hydrocarbyl, —O—C₁-C₄alkyl, methoxy, or mono-, di-, or tri-halo substituted alkyl),
Y is —NH₂ or OH;
Ar is arylene or heteroarylene, each of which is optionally substituted;
A is selected from the group consisting of a covalent bond, M¹-L²-M¹, and L²-M²-L² wherein
L², at each occurrence, is independently selected from the group consisting of a chemical bond, C₀-C₄ hydrocarbyl, C₀-C₄-hydrocarbyl-(NH)—C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-SO—C₀-C₄-hydrocarbyl, C₀-C₄-hydrocarbyl-SO₂—C₀-C₄-hydrocarbyl, C₀-C₄-hydrocarbyl-NH—CO—C₀-C₄-hydrocarbyl, and C₀-C₄-hydrocarbyl-CO—NH—C₀-C₄-hydrocarbyl, provided that L² is not a chemical bond when X¹ is M¹-L²-M¹;
M¹, at each occurrence, is independently selected from the group consisting of —O—, —N(R⁷)—, —S—, —S(O)—, S(O)₂—, —S(O)₂N(R⁷)—, —N(R⁷)—S(O)₂—, —C(O)—, —C(O)—NH—, —NH—C(O)—, —NH—C(O)—O— and —O—C(O)—NH—, wherein R⁷ is selected from the group consisting of hydrogen, alkyl, aryl, aralkyl, acyl, heterocyclyl, and heteroaryl; and
M² is selected from the group consisting of M¹, heteroarylene, and heterocyclylene, either of which rings optionally is substituted; and
L is selected from the group consisting of H, cycloalkyl, aryl, heteroaryl, or heterocyclyl, each of which is optionally substituted and each of which is optionally fused to one or more aryl or heteroaryl rings, or to one or more saturated or partially unsaturated cycloalkyl or heterocyclic rings, each of which rings is optionally substituted;
Formula (II) has the structure: 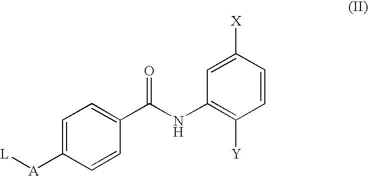
wherein
X is H, phenyl, thienyl, furanyl, pyridyl or pyrimidyl, each of which is optionally substituted;
Y is —NH₂;
A is —N(R⁷)—(CH₂)—; and
L is -heteroaryl-heteroaryl, -alkyl or heteroaryl, each of which is optionally substituted; wherein
R⁷ is selected from the group consisting of hydrogen, alkyl, aryl, aralkyl, acyl, heterocyclyl, and heteroaryl; and
Formula (III) has the structure: 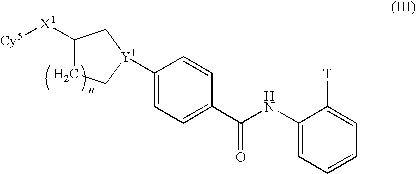
wherein
Cy⁵ is aryl, or heteroaryl, each of which is optionally substituted and wherein each of aryl and heteroaryl is optionally fused to one or more aryl or heteroaryl rings, or to one or more saturated or partially unsaturated cycloalkyl or heterocyclic rings, each of which rings is optionally substituted;
X¹ is selected from the group consisting of: a covalent bond, C₀-C₄-hydrocarbyl, C₀-C₄-hydrocarbyl-(CO)—C₀-C₄-hydrocarbyl, C₀-C₄-hydrocarbyl-N(R⁸)—C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-(SO)—C₀-C₄-hydrocarbyl, C₀-C₄-hydrocarbyl-(SO₂)-C₀-C₄-hydrocarbyl, C₀-C₄-hydrocarbyl-(NH)—(CO)—C₀-C₄-hydrocarbyl, C₀-C₄-hydrocarbyl -(CO)—(NH)—C₀-C₄-hydrocarbyl, —NH—CO—NH—, —NH—CS—NH—, —O—CO—O—, —O—CS—O—, —NH—C(NH)—NH—, —S(O)₂—N(R⁸)—, —N(R⁸)—S(O)₂—, —NH—C(O)—O—, and —O—C(O)—NH—;
wherein R⁸ is selected from the group consisting of hydrogen, C₁-C₅-alkyl, aryl, aralkyl, acyl, heterocyclyl, heteroaryl, SO₂-alkyl, SO₂-aryl, CO-alkyl, CO-aryl, CO—NH-alkyl, CO—NH-aryl, CO—O-alkyl and CO—O-aryl, each of which is optionally substituted;
n is 0 to 4;
Y¹ is N or CH; and
T is NH₂ or OH.

35. The method according to claim 33, wherein the selective inhibitor of HDAC1, HDAC2 and/or HDAC 3 has the structure

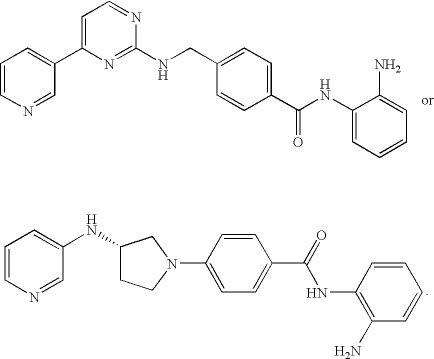

36. The method according to claim 33, wherein the compound that stabilizes microtubules is a taxane, an epothilone or an elpothilone analog.

37. The method according to claim 36, wherein the taxane is taxol or taxotere.

38. A method for controlling abnormal cell growth and/or abnormal cell proliferation in a patient, comprising administering to a patient in need thereof an effective amount of a selective inhibitor of histone deacetylase (HDAC)1, HDAC2 and/or HDAC3 in combination with an effective amount of a compound that stabilizes microtubules.

39. The method according to claim 38, wherein the selective inhibitor of HDAC1, HDAC2 and/or HDAC 3 has a structure represented by Formula (I), Formula (II) or Formula (III) and N-oxides, hydrates, solvates, pharmaceutically acceptable salts, prodrugs and complexes thereof, and racemic and scalemic mixtures, diastereomers, enantiomers and tautomers thereof, wherein
Formula (I) has the structure 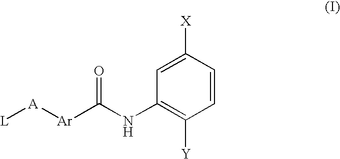
wherein
X is H, halo-, C₁-C₄-alkyl, C₁-C₄-alkoxy, —CH₂F, —CHF₂, —CF₃, aryl or heteroaryl, each of which is optionally substituted (preferably with one to three substituents independently selected from halo, —CN, —CH═N(OH), hydroxy, C₁-C₃-hydrocarbyl, —O—C₁-C₄alkyl, methoxy, or mono-, di-, or tri-halo substituted alkyl),
Y is —NH₂ or OH;
Ar is arylene or heteroarylene, each of which is optionally substituted;
A is selected from the group consisting of a covalent bond, M¹-L²-M¹, and L²-M²-L² wherein
L², at each occurrence, is independently selected from the group consisting of a chemical bond, C₀-C₄ hydrocarbyl, C₀-C₄-hydrocarbyl-(NH)—C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-SO—C₀-C₄-hydrocarbyl, C₀-C₄-hydrocarbyl-SO₂—C₀-C₄-hydrocarbyl, C₀-C₄-hydrocarbyl-NH—CO—C₀-C₄-hydrocarbyl, and C₀-C₄-hydrocarbyl-CO—NH—C₀-C₄-hydrocarbyl, provided that L² is not a chemical bond when X¹ is M¹-L²-M¹;
M¹, at each occurrence, is independently selected from the group consisting of —O—, —N(R⁷)—, —S—, —S(O)—, S(O)₂—, —S(O)₂N(R⁷)—, —N(R⁷)—S(O)₂—, —C(O)—, —C(O)—NH—, —NH—C(O)—, —NH—C(O)—O— and —O—C(O)—NH—, wherein R⁷ is selected from the group consisting of hydrogen, alkyl, aryl, aralkyl, acyl, heterocyclyl, and heteroaryl; and
M² is selected from the group consisting of M¹, heteroarylene, and heterocyclylene, either of which rings optionally is substituted; and
L is selected from the group consisting of H, cycloalkyl, aryl, heteroaryl, or heterocyclyl, each of which is optionally substituted and each of which is optionally fused to one or more aryl or heteroaryl rings, or to one or more saturated or partially unsaturated cycloalkyl or heterocyclic rings, each of which rings is optionally substituted;
Formula (II) has the structure: 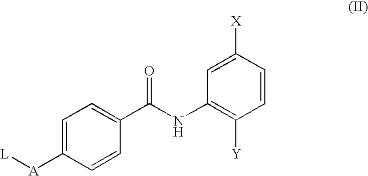
wherein
X is H, phenyl, thienyl, furanyl, pyridyl or pyrimidyl, each of which is optionally substituted;
Y is —NH₂;
A is —N(R⁷)—(CH₂)—; and
L is -heteroaryl-heteroaryl, -alkyl or heteroaryl, each of which is optionally substituted; wherein
R⁷ is selected from the group consisting of hydrogen, alkyl, aryl, aralkyl, acyl, heterocyclyl, and heteroaryl; and
Formula (III) has the structure: 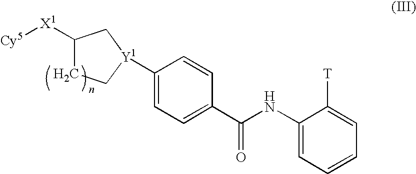
wherein
Cy⁵ is aryl, or heteroaryl, each of which is optionally substituted and wherein each of aryl and heteroaryl is optionally fused to one or more aryl or heteroaryl rings, or to one or more saturated or partially unsaturated cycloalkyl or heterocyclic rings, each of which rings is optionally substituted;
X¹ is selected from the group consisting of: a covalent bond, C₀-C₄-hydrocarbyl, C₀-C₄-hydrocarbyl-(CO)—C₀-C₄-hydrocarbyl, C₀-C₄-hydrocarbyl-N(R⁸)—C₀-C₄-hydrocarbyl, C₀-C₄-hydrocarbyl-(S)—C₀-C₄-hydrocarbyl, C₀-C₄-hydrocarbyl-(O)—C₀-C₄-hydrocarbyl, C₀-C₄-hydrocarbyl-(SO)—C₀-C₄-hydrocarbyl, C₀-C₄-hydrocarbyl-(SO₂)-C₀-C₄-hydrocarbyl, C₀-C₄-hydrocarbyl-(NH)—(CO)—C₀-C₄-hydrocarbyl, C₀-C₄-hydrocarbyl -(CO)—(NH)—C₀-C₄-hydrocarbyl, —NH—CO—NH—, —NH—CS—NH—, —O—CO—O—, —O—CS—O—, —NH—C(NH)—NH—, —S(O)₂—N(R⁸)—, —N(R⁸)—S(O)₂—, —NH—C(O)—O—, and —O—C(O)—NH—;
wherein R⁸ is selected from the group consisting of hydrogen, C₁-C₅-alkyl, aryl, aralkyl, acyl, heterocyclyl, heteroaryl, SO₂-alkyl, SO₂-aryl, CO-alkyl, CO-aryl, CO—NH-alkyl, CO—NH-aryl, CO—O-alkyl and CO—O-aryl, each of which is optionally substituted;
n is 0 to 4;
Y¹ is N or CH; and
T is NH₂ or OH.

40. The method according to claim 38, wherein the selective inhibitor of HDAC1, HDAC2 and/or HDAC 3 has the structure

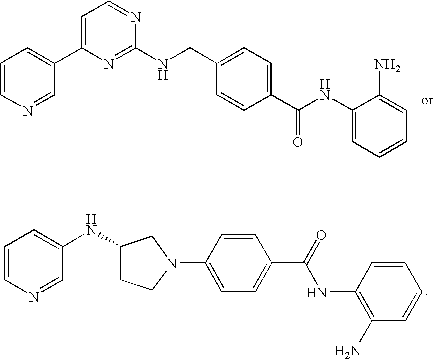

41. The method according to claim 38, wherein the compound that stabilizes microtubules is a taxane, an epothilone or an elpothilone analog.

42. The method according to claim 41, wherein the taxane is taxol or taxotere.

43. A method for controlling abnormal cell growth and/or abnormal cell proliferation in a patient, comprising administering to a patient in need thereof an effective amount of a selective inhibitor of histone deacetylase (HDAC)1 and/or HDAC2 in combination with an effective amount of a compound that stabilizes microtubules.

44. The method according to claim 43, wherein the selective inhibitor of HDAC1 and/or HDAC2 has a structure represented by Formula (IV), Formula (IVa) or Formula (V) and N-oxides, hydrates, solvates, pharmaceutically acceptable salts, prodrugs and complexes thereof, and racemic and scalemic mixtures, diastereomers, enantiomers and tautomers thereof, wherein
Formula (IV) has the structure. 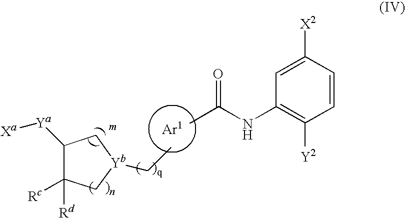
wherein
X² is aryl, cycloalkyl, heteroaryl or heterocyclyl, each of which is optionally substituted;
Ar¹ is aryl, heteroaryl, cycloalkyl or heterocyclyl, each of which is optionally substituted;
R^{a} is H or an optional substituent, preferably halo;
R^{b}, R^{c} and R^{d} are each independently hydrogen, C₁-C₈ alkyl, aryl, heteroaryl, cycloalkyl, heterocyclyl or halo; or
R^{b} and R^{c} together with the atoms to which they are bonded, optionally form a 5- or 6-membered cycloalkyl or heterocycloalkyl having 1 or 2 annular heteroatoms; each of which is optionally substituted with from 1 to 3 substituents;
Y² is —NH₂ or —OH;
Y^{b} is —N— or —CH—;
Y^{a} is a direct
bond, —O—, —N(R³⁴)—, —C(O)—, —OC(O)—, —C(O)O—, —N(R³⁴)—C(O)—, —C(O)—N(R³⁴)—, —N(R³⁴)—C(S)—, —C(S)—N(R³⁴), —N(R³⁴)—C(O)—N(R³)—, —N(R³⁴)—C(NR³⁴)—N(R³⁵)—, —N(R³⁴)—C(R³⁵)—, —C(NR³⁵) —N(R³⁴)—, —N(R³⁴)—C(S)—N(R³⁵)—, —N(R³⁴)—C(O)—O—, —O—C(O)—N(R³⁴)—, —N(R³⁴)—C(S)O—, —O—C(S) —N(R³⁵)—, —S(O)₀₋₂, —SO₂N(R³⁵)—, —N(R³⁵)—SO₂—N(R³⁴)—S(O)₂—N(R³⁵)—, —O—C₁-C₃alkyl-, —N(R³⁴)—C₁-C₃alkyl-, —C(O)—C₁-C₃alkyl- or —O—C(O)—C₁-C₃alkyl-;
X^{a} is C₁-C₈alkyl-, C₁-C₈alkenyl-, C₁-C₈alkynyl-, C₀-C₃alkyl-C₁-C₈alkenyl-C₀-C₃alkyl-, C₀-C₃alkyl-C₁-C₈alkynyl-C₀-C₃alkyl-, C₁-C₃alkyl-O—C₁-C₃alkyl-, HO—C₁-C₃alkyl-, C₁-C₄alkyl-N(R³⁴)—C₀-C₃alkyl-, N(R³⁴)(R³⁵)—C₀-C₃alkyl-, C₁-C₃alkyl-S(O)₀₋₂—C₁-C₃alkyl-, CF₃—C₀-C₃alkyl-, CF₂H—C₀-C₃alkyl-, C₁-C₈heteroalkyl-, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, aryl-C₀-C₂alkyl-heterocyclyl-C₀-C₂alkyl-, heteroaryl-C₀-C₂alkyl-heterocyclyl-C₀-C₂alkyl-, N(R³⁴)(R³⁵)-heterocyclyl-C₀-C₃alkyl-, heteroaryl-C₀-C₃alkyl-heterocyclyl- or C₁-C₄alkyl-CH(N(R³⁴)(R³⁵))—C(O)—N(R³⁴)-aryl-, wherein the aryl, cycloalkyl, heteroaryl and heterocycyl are optionally substituted with from 1 to 3 independently selected substituents;
or
X^{a}-Y^{a}- is selected from the group consisting of H—, halo-, HO—, HS—, HC(O)—, HOC(O)—, C₁-C₄alkyl-, H₂N—, (R³⁴)(R³⁵)N—, C₁-C₄alkyl-NH—, (C₁-C₄alkyl)₂-N—, HC(O)N(R³⁴)—, (R³⁴)(R³⁵)N—S(O)₂—N(R³⁶)—, (R³⁴)(R³⁵)N—C(O)—, H₂N—C(O)—, HC(S)N(R³⁴)—, (R³⁴)(R³⁵)N—C(S)—, H₂N—C(S)—, (R³⁴)(R³⁵)N—C(O)—O—, (R³⁴)(R³⁵)N—C(S)—O—, (R³⁴)(R³⁵)N—C(O)—N(R³⁶)—, (C₁-C₃alkylN)₂—C═N—, (R³⁴)(R³⁵)N—C(NR³⁷)—N(R³⁶), (R³⁴)(R³⁵)N—C(NR³⁶)—, cycloalkyl-C₀-C₂alkyl-C(NR³⁶)—, heterocyclyl-C₀-C₂alkyl-C(NR³⁶)—, aryl-C₀-C₂alkyl-C(NR³⁶)—, heteroaryl-C₀-C₂alkyl-C(NR³⁶)—, C₀-C₃alkyl-C(NR³⁶)—, C₁-C₄alkyl-S(O)₂—N(R³⁶)—, CF₃—C₀-C₄alkyl-S(O)₂—N(R³⁶)—, CF₃—C₀-C₄alkyl-C(O)—N(R³⁶)—, aryl-C₀-C₄alkyl-S(O)₂—N(R³⁶)—, heteroaryl-C₀-C₄alkyl-S(O)₂—N(R³⁶)—, cycloalkyl-C₀-C₄alkyl-S(O)₂—N(R³⁶)—, heterocyclyl-C₀-C₄alkyl-S(O)₂—N(R³⁶)—, C₁-C₄alkyl-O—C(O)—NH—, C₁-C₄alkyl-O—C(O)—N(H)—C₁-C₄alkyl-, C₁-C₄alkyl-N(H)—C(O)—N(H)—, C₁-C₄alkyl-NH—C(O)—O—, C₁-C₄alkyl-C(O)—N(H)—, C₁-C₄alkyl-O—C(S)—N(H)—, C₁-C₄alkyl-N(H)—C(S)—N(H)—, C₁-C₄alkyl-N(H)—C(S)—O—, C₁-C₄alkyl-C(S)—N(H)—, Me-C(O)—O—, Me-C(O)—N(H)—, aryl-C₀-C₄alkyl-O—C(O)—N(H)—, aryl-C₀-C₄alkyl-O—C(O)—N(C₁-C₄alkyl)-, aryl-C₀-C₄alkyl-C(O)—N(H)—, heteroaryl-C₀-C₄alkyl-O—C(O)—N(H)—, heteroaryl-C₀-C₄alkyl-O—C(O)—N(C₁-C₄alkyl)-, heteroaryl-C₀-C₄alkyl-C(O)—N(H)—, aryl-C₀-C₄alkyl-N(H)—C(O)—O—, heteroaryl-C₀-C₄alkyl-N(H)—C(O)—O—, heterocyclyl-C₀-C₄alkyl-O—C(O)—N(H)—, heterocyclyl-C₀-C₄alkyl-O—C(O)—N(C₁-C₄alkyl)-, heterocyclyl-C₀-C₄alkyl-C(O)—N(H)—, cycloalkyl-C₀-C₄alkyl-O—C(O)—N(H)—, cycloalkyl-C₀-C₄alkyl-O—C(O)—N(C₁-C₄alkyl)-, cycloalkyl-C₀-C₄alkyl-C(O)—N(H)—, heterocyclyl-C₀-C₄alkyl-N(H)—C(O)—O—, cycloalkyl-C₀-C₄alkyl-N(H)—C(O)—O—, heterocyclyl-C₀-C₄alkyl-C(O)—N(H)—, aryl-C₀-C₄alkyl-N(H)—C(O)—N(H)—, aryl-C₀-C₄alkyl-N(H)—, aryl-C₀-C₄alkyl-O—, aryl-C₀-C₄alkyl-S(O)₀₋₂—, heteroaryl-C₀-C₄alkyl-N(H)—C(O)—N(H)—, heteroaryl-C₀-C₄alkyl-N(H)—, heteroaryl-C₀-C₄alkyl-O—, heteroaryl-C₀-C₄alkyl-S(O)₀₋₂—, heterocyclyl-C₀-C₄alkyl-N(H)—C(O)—N(H)—, heterocyclyl-C₀-C₄alkyl-N(H)—, heterocyclyl-C₀-C₄alkyl-O—, heterocyclyl-C₀-C₄alkyl-S(O)₀₋₂—, cycloalkyl-C₀-C₄alkyl-N(H)—C(O)—N(H)—, cycloalkyl-C₀-C₄alkyl-N(H)—, cycloalkyl-C₀-C₄alkyl-O—, cycloalkyl-C₀-C₄alkyl-S(O)₀₋₂—, aryl-C₀-C₄alkyl-C(S)—N(H)—, heteroaryl-C₀-C₄alkyl-C(S)—N(H)—, aryl-C₀-C₄alkyl-O—C(S)—N(H)—, heteroaryl-C₀-C₄alkyl-O—C(S)—N(H)—, aryl-C₀-C₄alkyl-N(H)—C(S)—O—, heteroaryl-C₀-C₄alkyl-N(H)—C(S)—O—, heterocyclyl-C₀-C₄alkyl-C(S)—N(H)—, cycloalkyl-C₀-C₄alkyl-C(S)—N(H)—, heterocyclyl-C₀-C₄alkyl-O—C(S)—N(H)—, cycloalkyl-C₀-C₄alkyl-O—C(S)—N(H)—, heterocyclyl-C₀-C₄alkyl-N(H)—C(S)—O—, cycloalkyl-C₀-C₄alkyl-N(H)—C(S)—O—, heterocyclyl-C₀-C₄alkyl-C(S)—N(H)—, aryl-C₀-C₄alkyl-N(H)—C(S)—NH—, heteroaryl-C₀-C₄alkyl-N(H)—C(S)—N(H)—, heterocyclyl-C₀-C₄alkyl-N(H)—C(S)—N(H)—, cycloalkyl-C₀-C₄alkyl-N(H)—C(S)—N(H)—, C₁-C₄alkyl-O—C₁-C₄alkyl-C(O)—N(H)—, C₁-C₄alkyl-O—C₂-C₄alkyl-O—C(O)—N(H)—, C₁-C₄alkyl-O—C₂-C₄alkyl-N(H)—C(O)—N(H)—, C₁-C₄alkyl-O—C₂-C₄alkyl-N(H)—, C₁-C₄alkyl-O—C₂-C₄alkyl-O—, C₁-C₄alkyl-O—C₂-C₄alkyl-N(H)—C(O)—O—, HO—C₁-C₄alkyl-C(O)—N(H)—, HO—C₁-C₄alkyl-N(H)—, HO—C₁-C₄alkyl-N(R³)—, HO—C₁-C₄alkyl-O—, HO—C₁-C₄alkyl-S(O)₀₋₂—, HO—C₂-C₄alkyl-O—C(O)—N(H)—, HO—C₂-C₄alkyl-N(H)—C(O)—N(H)—, HO—C₂-C₄alkyl-N(H)—C(O)—O—, C₁-C₄alkyl-O—C₁-C₄alkyl-C(S)—N(H)—, C₁-C₄alkyl-O—C₂-C₄alkyl-O—C(S)—N(H)—, C₁-C₄alkyl-O—C₂-C₄alkyl-N(H)C(S)—N(H)—, C₁-C₄alkyl-O—C₂-C₄alkyl-N(H)—C(S)—O—, HO—C₂-C₄alkyl-O—C(S)—N(H)—, HO—C₂-C₄alkyl-N(H)—C(S)—N(H)—, HO—C₂-C₄alkyl-N(H)—C(S)—O—, (C₁-C₄alkyl)₂N—C₁-C₄alkyl-C(O)—N(H)—, (C₀-C₄alkyl)-O—C₁-C₄alkyl-C(O)—N(H)—, (C₀-C₄alkyl)-O—C₁-C₄alkyl-C(S)—N(H)—, (C₀-C₄alkyl)-O—C₁-C₄alkyl-C(O)—O—, (C₀-C₄alkyl)-O—C₂-C₄alkyl-N(H)—C(O)—N(H)—, (C₀-C₄alkyl)-O—C₂-C₄alkyl-O—C(O)—N(H)—, (C₀-C₄alkyl)-O—C₂-C₄alkyl-N(H)—C(NH)—N(H)—, (C₀-C₄alkyl)-O—C₂-C₄alkyl-N(H)—C(O)—, (C₁-C₄alkyl)₂N—C₂-C₄alkyl-O—C(O)—N(H)—, (C₁-C₄alkyl)₂N—C₂-C₄alkyl-N(H)—, (C₁-C₄alkyl)₂N—C₂-C₄alkyl-O—, (C₁-C₄alkyl)₂N—C₂-C₄alkyl-S(O)₀₋₂—, (C₁-C₄alkyl)₂N—C₂-C₄alkyl-N(H)—C(O)—N(H)—, (C₁-C₄alkyl)₂N—C₂-C₄alkyl-N(H)—C(O)—O—, (C₁-C₄alkyl)₂N—C₁-C₄alkyl-C(S)—N(H)—, (C₁-C₄alkyl)₂N—C₂-C₄alkyl-N(H)—C(S)—N(H)—, (C₁-C₄alkyl)₂N—C₂-C₄alkyl-N(H)—C(S)—O—, (C₁-C₄alkyl)-O—C(O)C₁-C₈alkyl-C(O)—(H)—, HO—C(O)C₁-C₈alkyl-C(O)—N(H)—, HO—NH—C(O)C₁-C₈alkyl-C(O)—N(H)—, CF₂H—C₀-C₄alkyl-C(O)—N(H)—, CF₃—C₀-C₄alkyl-C(O)—N(H)—, CF₃—C₀-C₄alkyl-N(H)—, CF₃—C₀-C₄alkyl-N(R³)—, CF₃—C₀-C₄alkyl-O—, CF₃—C₀-C₄alkyl-S(O)₀₋₂—, CF₃—C₀-C₄alkyl-O—C(O)—N(H)—, CF₃—C₀-C₄alkyl-N(H)C(O)—N(H)—, CF₃—C₀-C₄alkyl-N(H)—C(O)—O—, CF₃—C₀-C₄alkyl-O—C(S)—N(H)—, CF₃—C₀-C₄alkyl-N(H)—C(S)—N(H)—, CF₃—C₀-C₄alkyl-N(H)—C(S)—O—, CF₃—C₀-C₄alkyl-C(S)—N(H)—, CF₂H—C₀-C₄alkyl-N(H)—, CF₂H—C₀-C₄alkyl-O—, CF₂H-C₀-C₄alkyl-S(O)₀₋₂—, CF₂H—C₀-C₄alkyl-O—C(O)—N(H)—, CF₂H—C₀-C₄alkyl-N(H)C(O)—N(H)—, CF₂H—C₀-C₄alkyl-N(H)—C(O)—O—, CF₂H—C₀-C₄alkyl-O—C(S)—N(H)—, CF₂H—C₀-C₄alkyl-N(H)—C(S)—N(H)—, CF₂H—C₀-C₄alkyl-N(H)—C(S)—O—, CF₂H-C₀-C₄alkyl-C(S)—N(H)—, (H)(R³⁴)N—C₁-C₃alkyl-, (H)(R³⁴)N—C₁-C₃alkyl-, HO—C₁-C₃alkyl-, (H)(R³⁴)N—S(O)₂—N(R³⁵)—, (H)(R³⁵)N—S(O)₂—, (H)(R³⁴)N—C(S)—O—, (H)(R³⁴)N—C(O)—O—, (H)(R³⁴)N—C(S)—N(R³⁵)—, (H)(R³⁴)N—C(NR³⁵)—, (H)(R⁴)N—C(NR³⁴)—N(R³⁸)—, (H)(R³⁴)N—C(O)—N(R³⁵)—, HO—C(O)—C₁-C₃alkyl-, C₁-C₄alkyl-S(O)₂—NH— and ((R³⁴)(R³⁵)N)₂—C═N—;
m and n are independently 0, 1, 2 or 3;
q is 0, 1 or 2; and
R³⁴, R³⁵, R³⁶ and R³⁷ are each independently selected from the group consisting of hydrogen, cyano, oxo, hydroxyl, —C₁-C₈alkyl, C₁-C₈heteroalkyl, C₁-C₈alkenyl, carboxamido, C₁-C₃alkyl-carboxamido-, carboxamido-C₁-C₃alkyl-, amidino, C₂-C₈hydroxyalkyl, C₁-C₃alkylaryl-, aryl-C₁-C₃alkyl-, C₁-C₃alkylheteroaryl-, heteroaryl-C₁-C₃alkyl-, C₁-C₃alkylheterocyclyl-, heterocyclyl-C₁-C₃alkyl-, C₁-C₃alkylcycloalkyl-, cycloalkyl-C₁-C₃alkyl-, C₂-C₈alkoxy-, C₂-C₈alkoxy-C₁-C₄alkyl-, C₁-C₈alkoxycarbonyl-, aryloxycarbonyl-, aryl-C₁-C₃alkoxycarbonyl-, heteroaryloxycarbonyl-, heteroaryl-C₁-C₃alkoxycarbonyl-, C₁-C₈acyl, C₀-C₈alkyl-carbonyl-, aryl-C₀-C₈alkyl-carbonyl-, heteroaryl-C₀-C₈alkyl-carbonyl-, cycloalkyl-C₀-C₈alkyl-carbonyl-, C₀-C₈alkyl-N(H)-carbonyl-, aryl-C₀-C₈alkyl-N(H)-carbonyl-, heteroaryl-C₀-C₈alkyl-N(H)-carbonyl-, cycloalkyl-C₀-C₈alkyl-N(H)-carbonyl-, C₀-C₈alkyl-O-carbonyl-, aryl-C₀-C₈alkyl-O-carbonyl-, heteroaryl-C₀-C₈alkyl-O-carbonyl-, cycloalkyl-C₀-C₈alkyl-O-carbonyl-, C₁-C₈ alkylsulfonyl-, arylalkylsulfonyl-, arylsulfonyl-, heteroarylalkylsulfonyl-, heteroarylsulfonyl-, C₁-C₈alkyl-N(H)-sulfonyl-, arylalkyl-N(H)-sulfonyl-, aryl-N(H)-sulfonyl-, heteroarylalkyl-N(H)-sulfonyl-, heteroaryl-N(H)-sulfonyl, aroyl, aryl, cycloalkyl, heterocyclyl, heteroaryl, aryl-C₁-C₃alkyl-, cycloalkyl-C₁-C₃alkyl-, heterocyclyl-C₁-C₃ alkyl-, heteroaryl-C₁-C₃ alkyl-, and a protecting group, wherein each of the foregoing is further optionally substituted with one more moieties; or
R³⁴ and R³⁵ taken together with the N to which they are attached form a heterocyclyl or heteroaryl, each of which is optionally substituted with from 1 to 3 substituents, wherein the heterocyclyl may also be bridged (forming a bicyclic moiety with a methylene, ethylene or propylene bridge),
provided that 1) when Y^{b} is N, then m is not 0 if Y^{a} is bound to the ring comprising Y, via a N, S or O in Y^{a}, or 2) when m and n are both 0 then Y^{b} is —CH—;
Formula (IVa) has the structure: 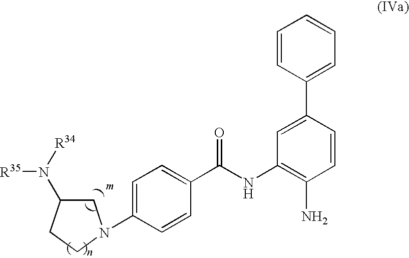
wherein m, n, R³⁴ and R³⁵ are as defined for formula (IV); and
Formula (V) has the structure: 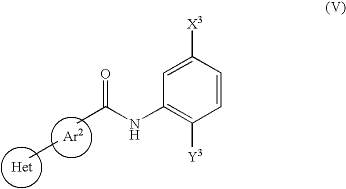
wherein
X³ is aryl, cycloalkyl, heteroaryl or heterocyclyl, each of which is optionally substituted;
Y³ is —NH₂ or —OH;
Ar² is optionally substituted aryl or optionally substituted heteroaryl; and
Het is an optionally substituted heterocyclyl.

45. The method according to claim 44, wherein the selective inhibitor of HDAC1 and/or HDAC2 has the structure

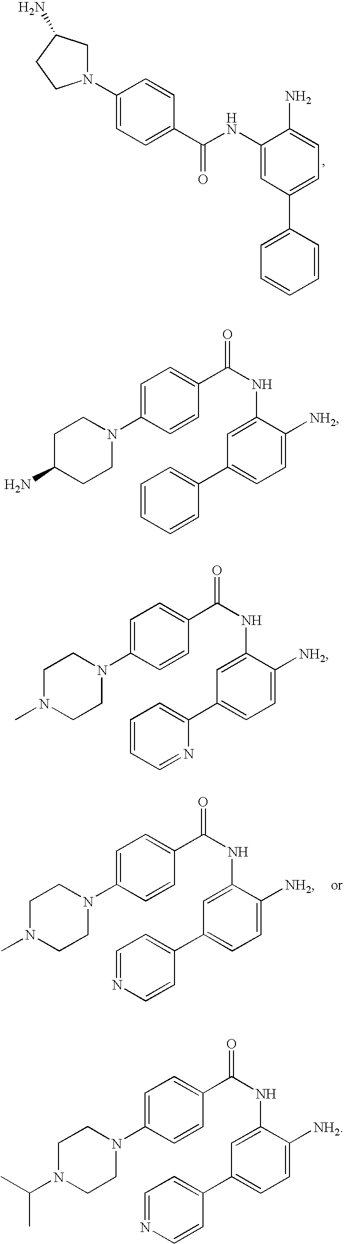

46. The method according to claim 43, wherein the compound that stabilizes microtubules is a taxane, an epothilone or an elpothilone analog.

47. The method according to claim 46, wherein the taxane is taxol or taxotere.

48. Use of a selective inhibitor of histone deacetylase (HDAC)1, HDAC2 and/or HDAC3, in combination with a compound that stabilizes microtubules for the manufacture of a medicament to inhibit abnormal cell growth and/or abnormal cell proliferation, or to treat cancer in a patient.
